SEQUENCE LISTING (27 sequences)

SEQ ID NO: 1 - protein, 574 aa, synthetic construct
PTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSMAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPST

SEQ ID NO: 2 - protein, 459 aa, Homo sapiens
DQESCKGRCTEGFNVDKKCQCDELCSYYQSCCTDYTAECKPQVTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSRATWLSLFSSEESNLGANNYDDYRMDWLVPATCEPIQSVFFFSGDKYYRVNLRTRRVDTVDPPYPRSIAQYWLGCPAPGHL

SEQ ID NO: 3 - protein, 478 aa, Homo sapiens
MAPLRPLLILALLAWVALADQESCKGRCTEGFNVDKKCQCDELCSYYQSCCTDYTAECKPQVTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSRATWLSLFSSEESNLGANNYDDYRMDWLVPATCEPIQSVFFFSGDKYYRVNLRTRRVDTVDPPYPRSIAQYWLGCPAPGHL

SEQ ID NO: 4-5 - skipped (2 entries)

SEQ ID NO: 6 - protein, 25 aa, synthetic construct
CCTDYTAECKPQVTRGDVFTMPEDE

SEQ ID NO: 7 - protein, 19 aa, synthetic construct
CCRRPQVTRGDVFTMPWLC

SEQ ID NO: 8 - protein, 12 aa, synthetic construct
PQVTRGDVFTMP

SEQ ID NO: 9 - protein, 19 aa, synthetic construct
KKALLILALLAHWVALAKK

SEQ ID NO: 10 - protein, 13 aa, synthetic construct
LLILALLAWVALA

SEQ ID NO: 11 - protein, 14 aa, synthetic construct
LLILALLAHWVALA

SEQ ID NO: 12 - protein, 34 aa, synthetic construct
RPSLAKKQRFRHRNRKGYRSQRGHSRGRNQNSRR

SEQ ID NO: 13 - protein, 23 aa, synthetic construct
DYTITVYAVTGRGDSPASSKPIS

SEQ ID NO: 14 - protein, 179 aa, synthetic construct
PAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDAST

SEQ ID NO: 15 - protein, 90 aa, synthetic construct
PAPTDLKFTQVTPTSLSAQWTPPNVQLTGYRVRVTPKEKTGPMKEINLAPDSSSVVVSGLMVATKYEVSVYALKDTLTSRPAQGVVTTLE

SEQ ID NO: 16 - protein, 392 aa, synthetic construct
MAPLRPLLILALLAWVALADQESCKGRCTEGFNVDKKCQCDELCSYYQSCCTDYTAECKPQVTRGDVFTMPAPTDLKFTQVTPTSLSAQWTPPNVQLTGYRVRVTPKEKTGPMKEINLAPDSSSVVVSGLMVATKYEVSVYALKDTLTSRPAQGVVTTLEEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 481 aa, synthetic construct
MAPLRPLLILALLAWVALADQESCKGRCTEGFNVDKKCQCDELCSYYQSCCTDYTAECKPQVTRGDVFTMPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 249 aa, synthetic construct
MAPLRPLLILALLAWVALADQESCKGRCTEGFNVDKKCQCDELCSYYQSCCTDYTAECKPQVTRGDVFTMPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDAST

SEQ ID NO: 19 - protein, 178 aa, synthetic construct
APTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDAST

SEQ ID NO: 20 - protein, 89 aa, synthetic construct
APTDLKFTQVTPTSLSAQWTPPNVQLTGYRVRVTPKEKTGPMKEINLAPDSSSVVVSGLMVATKYEVSVYALKDTLTSRPAQGVVTTLE

SEQ ID NO: 21 - protein, 71 aa, synthetic construct
MAPLRPLLILALLAWVALADQESCKGRCTEGFNVDKKCQCDELCSYYQSCCTDYTAECKPQVTRGDVFTMP

SEQ ID NO: 22 - protein, 19 aa, synthetic construct
MAPLRPLLILALLAWVALA

SEQ ID NO: 23 - protein, 52 aa, synthetic construct
DQESCKGRCTEGFNVDKKCQCDELCSYYQSCCTDYTAECKPQVTRGDVFTMP

SEQ ID NO: 24 - protein, 24 aa, synthetic construct
LAQHEQGGFNNNFNTSNWSSAQVN

SEQ ID NO: 25 - protein, 26 aa, synthetic construct
KKALLAHALHLLALLALHLAHALKKA

SEQ ID NO: 26 - protein, 23 aa, synthetic construct
PLLILALLAWVALADQESCKGRC

SEQ ID NO: 27 - protein, 5 aa, synthetic construct
EPKSA

(19) United States
(12) Patent Application Publication
(10) Pub. No.: US 2026/0308612 A1
(43) Pub. Date: Oct. 8, 2026

(54) PRODUCTION OF CELLS AND VIRAL VECTORS

(71) Applicant: BIOCELL INNOVATIONS PTE., LTD., Singapore (SG)

(72) Inventors: Paula Lam, Singapore (SG); Mobashar Hussain Urf Turabe Fazil, Singapore (SG)

(73) Assignee: BIOCELL INNOVATIONS PTE., LTD., Singapore (SG)

(21) Appl. No.: 18/998,452

(22) PCT Filed: Jul. 27, 2023
(86) PCT No.: PCT/EP2023/070888

§ 371 Date: Jan. 24, 2025

(30) Foreign Application Priority Data
Jul. 27, 2022 (SG) 10202250599M

(51) Int. Cl. C12N 15/86; A61K 40/11; A61K 40/31; C07K 16/28; C12N 5/0783

(52) U.S. Cl. CPC C12N 15/86; A61K 40/11; A61K 40/31; C07K 16/2803; C12N 5/0636; C07K 2317/24; C12N 2510/00; C12N 2740/15043; C12N 2740/15052

(57) ABSTRACT

Disclosed herein are cell culture methods, methods of producing a cell for use in adoptive cell therapy, methods of transducing an immune cell with a viral vector, and uses of products derived from the methods e.g. medical uses and therapeutic applications.

This application claims priority from SG 10202250599M filed 27 Jul. 2022, the contents and elements of which are herein incorporated by reference for all purposes.

### FIELD OF THE INVENTION

The present invention relates to molecular and cell biology and particularly, although not exclusively, to methods of culturing cells and producing viral vectors.

### BACKGROUND

Viral vectors, such as lentiviral vectors (LV), are emerging as a powerful tool in gene and cell therapies due to their abilities to deliver long-term transgene expression in both dividing and non-dividing cells. There are currently either adherent or suspension-based viral vectors manufacturing processes, and each approach has positive and negative aspects. At present, 70% of viral vector products are manufactured using adherent culture processes where cells are grown as monolayers in culture vessels instead of free-floating in culture media (i.e., suspension). Adherent-based manufacturing processes can be readily procured off-the-shelf and require less expert bioengineering know-how compared to three-dimensional platforms. However, most adherent processes are dependent on products of animal origin like fetal bovine serum (FBS) that pose safety, consistency, and regulatory challenges.

Poly-L-lysine is a non-specific attachment factor for cell adhesion to culture surfaces through the increase of positively charged cell binding sites on polystyrene culture surface. This enhances the electrostatic interaction between negatively charged ions of the cell membrane and the culture-ware. Because HEK293 or derived cells are easily detached from the cell culture vessels, several lentiviral production protocols suggest coating the culture-ware with poly-L-lysine to enhance cell attachment. For example, Gandara and colleagues have seeded HEK293T cells in 10% serum containing DMEM culture dishes pre-coated with poly-L-lysine for the production of lentiviral vectors (Gandara et al., Hum Gene Ther Methods. (2018) 29(1):1-15). However, serum is still required and poly-L-lysine has not been shown to enhance transduction efficiency in peripheral blood mononuclear cells (PBMCs).

RetroNectin® is a recombinant human fibronectin fragment that is used as a gold standard transduction enhancer for retroviral and lentiviral gene transfer to hematopoietic cells. RetroNectin has been adopted globally in various clinical trials including CAR-LV T cell transduction.

Vitronectin binds to plasminogen activator inhibitor-1 (PAI-1) and stabilizes it (Zhou et al. Nature Structural Biology. (2003) 10(7):541-4.). Thus, vitronectin serves to regulate proteolysis initiated by plasminogen activation. It has been found to support the growth and differentiation of human pluripotent stem cells.

### SUMMARY OF THE INVENTION

In one aspect, the present invention provides a cell culture method, the method comprising adherent culture of cells in a culture vessel, wherein the cells are cultured in culture media that is a fetal bovine serum (FBS)-free media and the culture vessel contains a polypeptide having at least 70% sequence identity to one of SEQ ID NO:1, SEQ ID NO:2, or SEQ ID NO:3.

The present invention also provides a cell culture method, the method comprising adherent culture of cells in a culture vessel, wherein the cells are cultured in culture media that is a fetal bovine serum (FBS)-free media and the culture vessel contains a polypeptide comprising an amino acid sequence having at least 70% sequence identity to one of SEQ ID NO:1, SEQ ID NO:2, or SEQ ID NO:3, or a polypeptide comprising an amino acid sequence having at least 70% sequence identity to a fragment of SEQ ID NO:1, SEQ ID NO:2, or SEQ ID NO:3.

In another aspect, the present invention provides the use of a polypeptide having at least 70% sequence identity to one of SEQ ID NO:1, SEQ ID NO:2, or SEQ ID NO:3 in a method of producing a viral vector, wherein the method comprises the step of culturing a cell in FBS-free media within a culture vessel.

The present invention also provides the use of a polypeptide having at least 70% sequence identity to one of SEQ ID NO:1, SEQ ID NO:2, or SEQ ID NO:3, or a polypeptide comprising an amino acid sequence having at least 70% sequence identity to a fragment of SEQ ID NO:3, SEQ ID NO:2, or SEQ ID NO:1, in a method of producing a viral vector, wherein the method comprises the step of culturing a cell in FBS-free media within a culture vessel.

In some embodiments, the polypeptide used in the method enhances the adhesion of the cells to the culture vessel.

In some embodiments, the polypeptide comprising an amino acid sequence having at least 70% sequence identity to a fragment of SEQ ID NO:3, SEQ ID NO:2, or SEQ ID NO:1 comprises at least 70% sequence identity to one or more of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, and/or SEQ ID NO:23, preferably wherein the polypeptide comprises one of at least 80%, 85%, 90%, 95%, or 99% sequence identity to one or more of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, and/or SEQ ID NO:23.

In some embodiments, the polypeptide comprising an amino acid sequence having at least 70% sequence identity to a fragment of SEQ ID NO:3, SEQ ID NO:2, or SEQ ID NO:1 comprises at least 70% sequence identity to one or more of SEQ ID NO:6, SEQ ID NO:7, SEQ ID NO:8, SEQ ID NO:9, SEQ ID NO:10, SEQ ID NO:11, SEQ ID NO:12, SEQ ID NO:16, SEQ ID NO:17, SEQ ID NO:18, SEQ ID NO:19, SEQ ID NO:20, SEQ ID NO:21, SEQ ID NO:22, and/or SEQ ID NO:23, preferably wherein the polypeptide has one of at least 80%, 85%, 90%, 95%, or 99% sequence identity to one or more of SEQ ID NO:6, SEQ ID NO:7, SEQ ID NO:8, SEQ ID NO:9, SEQ ID NO:10, SEQ ID NO:11, SEQ ID NO:12, SEQ ID NO:16, SEQ ID NO:17, SEQ ID NO:18, SEQ ID NO:19, SEQ ID NO:20, SEQ ID NO:21, SEQ ID NO:22, and/or SEQ ID NO:23.

In some embodiments, the polypeptide comprising an amino acid sequence having at least 70% sequence identity to a fragment of SEQ ID NO:3, SEQ ID NO:2, or SEQ ID NO:1 comprises at least 70% sequence identity to one or more of SEQ ID NO:6, SEQ ID NO:7, SEQ ID NO:8, SEQ ID NO:9, SEQ ID NO:10, SEQ ID NO:11, SEQ ID NO:12, SEQ ID NO:16, and/or SEQ ID NO:17, preferably wherein the polypeptide comprises one of at least 80%, 85%, 90%, 95%, or 99% sequence identity to one or more of SEQ ID NO:6, SEQ ID NO:7, SEQ ID NO:8, SEQ ID NO:9, SEQ ID NO:10, SEQ ID NO:11, SEQ ID NO:12, SEQ ID NO:16, and/or SEQ ID NO:17.

In some embodiments, the polypeptide comprising an amino acid sequence having at least 70% sequence identity to a fragment of SEQ ID NO:3, SEQ ID NO:2, or SEQ ID NO:1 comprises at least 70% sequence identity to one or more of SEQ ID NO:6, SEQ ID NO:7, SEQ ID NO:9, SEQ ID NO:12, SEQ ID NO:16, and/or SEQ ID NO:17, preferably wherein the polypeptide comprises one of at least 80%, 85%, 90%, 95%, or 99% sequence identity to one or more of SEQ ID NO:6, SEQ ID NO:7, SEQ ID NO:9, SEQ ID NO:12, SEQ ID NO:16, and/or SEQ ID NO:17.

In some embodiments, the polypeptide comprising an amino acid sequence having at least 70% sequence identity to a fragment of SEQ ID NO:3 comprises at least 70% sequence identity to one or more of SEQ ID NO:6, SEQ ID NO:7, SEQ ID NO:9, and/or SEQ ID NO:12, preferably wherein the polypeptide comprises one of at least 80%, 85%, 90%, 95%, or 99% sequence identity to one or more of SEQ ID NO:6, SEQ ID NO:7, SEQ ID NO:9, and/or SEQ ID NO:12.

In some embodiments, one or more of the cells expresses a viral vector.

In some embodiments, the viral vector is a lentiviral vector, retroviral vector, adeno-associated viral (AAV) vector, or adenoviral vector.

In some embodiments, the cell is a mammalian or insect cell. In some embodiments, the mammalian cell is a human cell. In some embodiments, the human cell is a human embryonic kidney cell, HeLa cell, or HT-1080 cell. In some embodiments, the human embryonic kidney cell is a HEK293 cell, HEK293T cell, or HEK293FT cell.

In some embodiments, the polypeptide has at least 75% sequence identity to one of SEQ ID NO:1, SEQ ID NO:2, or SEQ ID NO:3, preferably wherein the polypeptide has one of at least 80%, 85%, 90%, 95%, or 99% sequence identity to one of SEQ ID NO:1, SEQ ID NO:2, or SEQ ID NO:3. In some embodiments, the polypeptide has the sequence of SEQ ID NO:1, SEQ ID NO:2, or SEQ ID NO:3, i.e. 100% sequence identity to one of SEQ ID NO:1, SEQ ID NO:2, or SEQ ID NO:3.

In some embodiments, the polypeptide is vitronectin or RetroNectin®. In some embodiments, the polypeptide is a fragment of vitronectin or RetroNectin®. In some embodiments, the polypeptide is a functional fragment of vitronectin or RetroNectin®. In some embodiments, the polypeptide is a fragment of vitronectin or RetroNectin® capable of enhancing adhesion of cells to a culture vessel, and/or enhancing viral transduction. In some embodiments, the polypeptide is a fragment of vitronectin or RetroNectin® capable of enhancing adhesion of cells to a culture vessel, and/or enhancing viral transduction, and/or reducing cytokine secretion from a cell, and/or reducing cytokine secretion from a cell whilst maintaining the killing of target cells, and/or increasing killing of target cells by a cell for adoptive cell transfer.

In some embodiments, the polypeptide is vitronectin. In some embodiments, the polypeptide is a fragment of vitronectin. In some embodiments, the polypeptide is derived from vitronectin. In some embodiments, the polypeptide is a functional fragment of vitronectin. In some embodiments, the polypeptide is a fragment of vitronectin capable of enhancing adhesion of cells to a culture vessel, enhancing viral transduction, reducing cytokine secretion from a cell, and/or reducing cytokine secretion from a cell whilst maintaining the killing of target cells, and/or increasing killing of target cells by a cell.

In some embodiments, the polypeptide is RetroNectin®. In some embodiments, the polypeptide is a fragment of RetroNectin®. In some embodiments, the polypeptide is derived from RetroNectin®. In some embodiments, the polypeptide is a functional fragment of RetroNectin®. In some embodiments, the polypeptide is a fragment RetroNectin® capable of enhancing adhesion of cells to a culture vessel, enhancing viral transduction, reducing cytokine secretion from a cell, and/or reducing cytokine secretion from a cell whilst maintaining the killing of target cells, and/or increasing killing of target cells by a cell.

In some embodiments, the cytokine is one or more of IL-6, TNF-α, IFN-γ, and IL-8. In some embodiments, the cytokine is IL-6. In some embodiments, the cytokine is TNF-α. In some embodiments, the cytokine is IFN-γ. In some embodiments, the cytokine is IL-8.

In some embodiments, the polypeptide has at least 75% sequence identity to one of SEQ ID NO:1, SEQ ID NO:2, or SEQ ID NO:3, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, and/or SEQ ID NO:23, preferably wherein the polypeptide has one of at least 80%, 85%, 90%, 95%, or 99% sequence identity to one of SEQ ID NO:1, SEQ ID NO:2, or SEQ ID NO:3, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, and/or SEQ ID NO:23.

In some embodiments, the polypeptide improves cell adhesion compared to the cell adhesion of control cells cultured in the same way but without the polypeptide.

In some embodiments, the polypeptide improves cell adhesion compared to the cell adhesion of control cells cultured in the same way but without the polypeptide, as assessed by microscopy, a flow chamber pressure assay, a non-invasive and label-free real-time impedance assay, or through an assay utilising anti-cell adhesion antibodies.

In some embodiments, the polypeptide reduces cellular aggregation compared to the cellular aggregation of control cells cultured in the same way but without the polypeptide, as assessed by microscopy.

In some embodiments, the polypeptide increases cell distribution in the culture vessel compared to the distribution of control cells cultured in the same way but without the polypeptide, as assessed by microscopy.

In some embodiments, the polypeptide increases viral titre compared to the viral titre produced by control cells cultured in the same way but without the polypeptide.

In some embodiments, the viral titre is increased by over 1.5 times when the polypeptide is used compared to the viral titre produced by control cells cultured in the same way but without the polypeptide.

In some embodiments, the viral titre is increased by over 2 times when the polypeptide is used compared to the viral titre produced by control cells cultured in the same way but without the polypeptide. preferably wherein viral titre is increased by one of over 3 times, 4 times, 5 times, 6 times, or 7 times when the polypeptide is used compared to the viral titre produced by control cells cultured in the same way but without the polypeptide.

In some embodiments, the polypeptide is provided as a liquid solution. In some embodiments, the polypeptide is present in the culture.

In some embodiments, the polypeptide is present in the culture at a concentration of at least 0.1 μg/mL of the total culture. In some embodiments, the polypeptide is present in the culture at a concentration of at least 0.1 μg/mL of the total culture, preferably wherein the polypeptide is added at a concentration of at least 0.5 μg/mL, 1 μg/mL, 2 μg/mL, 3 μg/mL, 4 μg/mL, 5 μg/mL, 10 μg/mL, 15 μg/mL, or 20 μg/mL.

In some embodiments, the polypeptide is added to the culture. In some embodiments, the polypeptide is added to the culture as a liquid solution. In some embodiments, the polypeptide is added at a concentration of at least 0.1 μg/mL of the total culture. In some embodiments, the polypeptide is added at a concentration of at least 0.1 μg/mL of the total culture, preferably wherein the polypeptide is added at a concentration of at least 0.5 μg/mL, 1 μg/mL, 2 μg/mL, 3 μg/mL, 4 μg/mL, 5 μg/mL, 10 μg/mL, 15 μg/mL, or 20 μg/mL.

In some embodiments, the culture vessel comprises a coating. In some embodiments, the polypeptide is coated onto a surface of the culture vessel. In some embodiments, the polypeptide is coated onto an inner/internal surface of the culture vessel. In some embodiments, the culture surface comprises a coating.

In some embodiments, the culture vessel coating comprises at least 10% vitronectin and/or RetroNectin®.

In some embodiments, the culture vessel coating comprises one of at least 20%, 30%, 40%, 50%, 60%, 70%, 80%, 90%, 95%, 96%, 97%, 98%, or 99% vitronectin and/or RetroNectin®. In some embodiments, the culture vessel coating is 100% vitronectin and/or RetroNectin®.

In some embodiments, at least 10% of the culture vessel coating comprises a polypeptide having at least 70% sequence identity to SEQ ID NO:3, SEQ ID NO:2, or SEQ ID NO:1. In some embodiments, at least 20%, 30%, 40%, 50%, 60%, 70%, 80%, 90%, 95%, 96%, 97%, 98%, or 99% of the culture vessel coating comprises a polypeptide having at least 70% sequence identity to SEQ ID NO:3, SEQ ID NO:2, or SEQ ID NO:1.

In some embodiments, at least 10% of the culture vessel coating comprises a polypeptide having at least 70% sequence identity to SEQ ID NO:1, SEQ ID NO:2, or SEQ ID NO:3 or a polypeptide comprising an amino acid sequence having at least 70% sequence identity to a fragment of SEQ ID NO:3, SEQ ID NO:2, or SEQ ID NO:1. In some embodiments, at least 20%, 30%, 40%, 50%, 60%, 70%, 80%, 90%, 95%, 96%, 97%, 98%, or 99% of the culture vessel coating comprises a polypeptide having at least 70% sequence identity to SEQ ID NO:1, SEQ ID NO:2, or SEQ ID NO:3 or a polypeptide comprising an amino acid sequence having at least 70% sequence identity to a fragment of SEQ ID NO:3, SEQ ID NO:2, or SEQ ID NO:1.

In some embodiments, the culture vessel coating consists of a polypeptide having at least 70% sequence identity to SEQ ID NO:3, SEQ ID NO:2, or SEQ ID NO:1.

In some embodiments, the culture vessel coating consists of a polypeptide having at least 70% sequence identity to SEQ ID NO:3, SEQ ID NO:2, or SEQ ID NO:1 or a polypeptide comprising an amino acid sequence having at least 70% sequence identity to a fragment of SEQ ID NO:3, SEQ ID NO:2, or SEQ ID NO:1.

In some embodiments, the polypeptide is coated onto the culture vessel before media is added. In some embodiments, the culture vessel is coated by adding a solution comprising the polypeptide to the culture vessel, incubating the culture vessel with the solution inside the vessel, and discarding the remaining solution after incubation. In some embodiments, the incubation is for at least 1 hour. In some embodiments, the incubation is for one of at least 2, 3, 4, 5, 6, 7, 8, 9, 10, 11, 12, 13, 14, 15, 16, 17, 18, 19, 20, 21, 22, 23, or 24 hours. In some embodiments, the incubation is for one of at least 1, 2, 3, 4, 5, 6, 7, 8, 9, 10, 11, 12, 13, 14, 15, 16, 17, 18, 19, 20, 21, 22, 23, 24, 25, 26, 27, or 28 day(s).

In some embodiments, the incubation is performed in a refrigerator, at room temperature, or in an incubator at ≥30° C. In some embodiments, the solution comprising the polypeptide contains the polypeptide at a concentration of at least 0.1 μg/mL. In some embodiments, the solution comprising the polypeptide contains the polypeptide at a concentration of at least 0.5 μg/mL, 1 μg/mL, 2 μg/mL, 3 μg/mL, 4 μg/mL, 5 μg/mL, 10 μg/mL, 15 μg/mL, or 20 μg/mL.

In some embodiments, the culture vessel is a culture plate, a culture dish, a culture flask, a culture bottle, or a bioreactor. In some embodiments, the culture plate is a 2-well plate, 4-well plate, a 6-well plate, a 12-well plate, a 24-well plate, a 48-well plate, a 96-well plate, or a 384-well plate.

In some embodiments, the culture vessel has a volume of at least 0.2 mL. In some embodiments, the culture vessel has a volume of at least 0.5 mL, preferably wherein the culture vessel has a volume of at least 0.6, 0.7, 0.8, 0.9, 1.5, 1.0, 2.0, 3.0, 4.0, 5.0, 10.0, 15.0, 20.0, 25.0, 30.0, 35.0, 40.0, 45.0, 50.0, 60.0, 70.0, 80.0, 90.0, 100.0, 125.0, 150.0, 175.0, or 200.0 mL.

In some embodiments, the polypeptide is provided as an additive to commercially available FBS-free media. In some embodiments, the culture media does not contain animal serum. In some embodiments, the culture media is serum-free, xeno-free and/or animal product-free.

In some embodiments, the polypeptide having at least 70% sequence identity to SEQ ID NO:3, SEQ ID NO:2, or SEQ ID NO:1 is added to protein-free media.

In some embodiments, the polypeptide having at least 70% sequence identity to SEQ ID NO:3, SEQ ID NO:2, or SEQ ID NO:1, or a polypeptide comprising an amino acid sequence having at least 70% sequence identity to a fragment of SEQ ID NO:3, SEQ ID NO:2, or SEQ ID NO:1, is added to protein-free media.

In some embodiments, the cells have been transfected with a packaging helper plasmid. In some embodiments, the cells have been transfected with three packaging helper plasmids. In some embodiments, the cells have been transfected with a packaging helper plasmid which carries a HIV1 Rev protein, a packaging helper plasmid which carries an envelope protein, and/or a packaging helper plasmid which carries HIV1 gag/pol proteins. In some embodiments, the envelope protein is a vesicular stomatitis virus G (VSV-G) protein, a Baboon protein (e.g. BaEV), a RD114 protein, a measles virus protein (e.g. H/F), a cocal virus protein (e.g. COCV), a nipah virus protein, an Ebola protein, a Mokola protein, a LCMV protein, and/or a MuLV glycoprotein. In some embodiments, the cells have been transfected with a plasmid encoding a gene of interest.

In some embodiments, the method further comprises a step of transducing cells with a packaging helper plasmid and a plasmid containing a gene of interest before the cells are cultured. In some embodiments, the gene of interest is a chimeric antigen receptor (CAR) gene.

In another aspect, the present invention provides a method of producing a viral vector, the method comprising culturing cells according to the method of culturing cells as discussed above, the method further comprising the step of isolating or collecting a virus, a viral vector, and/or a viral particle from the culture.

In another aspect, the present invention provides a method of producing a cell for use in adoptive cell therapy comprising the following steps:
(i) producing a viral vector using the method described above, and
(ii) transducing an immune cell with the viral vector to generate a cell for use in adoptive cell therapy.

In some embodiments, the cell for use in adoptive cell therapy is a CAR-expressing immune cell. In some embodiments, the cell for use in adoptive cell therapy is a CAR T-cell or CAR-Natural Killer (CAR-NK) cell.

In some embodiments, the immune cell is a T-cell or NK cell. In some embodiments, the immune cell is a mammalian T-cell or NK cell. In some embodiments, the immune cell is a human T-cell or NK cell. In some embodiments, the T-cell is a gamma delta (γδ) T cell, a cytotoxic T cell, or a helper T cell.

In some embodiments, the viral vector is a lentiviral vector, retroviral vector, Adeno-Associated Viral (AAV) vector, or adenoviral vector.

In some embodiments, the method steps (i) and (ii) are performed without changing media. In some embodiments, a common FBS-free media is used in steps (i) and (ii).

In some embodiments, step (ii) is performed in the presence of a polypeptide having at least 70% sequence identity to one of SEQ ID NO:3, SEQ ID NO:2 or SEQ ID NO:1.

In some embodiments, step (ii) is performed in the presence of a polypeptide having at least 70% sequence identity to one of SEQ ID NO:3, SEQ ID NO:2 or SEQ ID NO:1, or a polypeptide comprising an amino acid sequence having at least 70% sequence identity to a fragment of SEQ ID NO:3, SEQ ID NO:2, or SEQ ID NO:1.

In some embodiments, a polypeptide having at least 70% sequence identity to SEQ ID NO:3, SEQ ID NO:2 or SEQ ID NO:1 is used in step (ii) to enhance transduction.

In some embodiments, a polypeptide having at least 70% sequence identity to SEQ ID NO:3, SEQ ID NO:2 or SEQ ID NO:1, or a polypeptide comprising an amino acid sequence having at least 70% sequence identity to a fragment of SEQ ID NO:3, SEQ ID NO:2, or SEQ ID NO:1, is used in step (ii) to enhance transduction.

In some embodiments, the polypeptide is further used in an additional method step to enhance transduction of a human cell with the viral vector to generate a cell for use in adoptive cell therapy.

In another aspect, the present invention provides a method of producing a cell for adoptive cell therapy, the method comprising contacting the cell with a viral vector encoding the gene of interest and a polypeptide comprising an amino acid sequence having at least 70% sequence identity to one of SEQ ID NO:3, SEQ ID NO:2 or SEQ ID NO:1, or a polypeptide comprising an amino acid sequence having at least 70% sequence identity to a fragment of SEQ ID NO:3, SEQ ID NO:2, or SEQ ID NO:1.

In another aspect, the present invention provides a method of introducing nucleic acids into mammalian cells using viral-mediated gene transduction, wherein a polypeptide having at least 70% sequence identity to SEQ ID NO:3 or SEQ ID NO:2 is employed as an enhancer of viral-mediated gene transduction.

In another aspect, the present invention provides a method of introducing nucleic acids into mammalian cells using viral-mediated gene transduction, wherein a polypeptide having at least 70% sequence identity to SEQ ID NO:3 or SEQ ID NO:2, or a polypeptide comprising an amino acid sequence having at least 70% sequence identity to a fragment of SEQ ID NO:3 or SEQ ID NO:2, is employed as an enhancer of viral-mediated gene transduction.

In another aspect, the present invention provides the use of a polypeptide having at least 70% sequence identity to SEQ ID NO:3 or SEQ ID NO:2 as an enhancer of viral-mediated gene transduction of mammalian cells.

In another aspect, the present invention provides the use of a polypeptide having at least 70% sequence identity to SEQ ID NO:3 or SEQ ID NO:2, or a polypeptide comprising an amino acid sequence having at least 70% sequence identity to a fragment of SEQ ID NO:3 or SEQ ID NO:2, as an enhancer of viral-mediated gene transduction of mammalian cells.

In another aspect, the present invention provides a method of transducing an immune cell with a viral vector encoding a gene of interest, the method comprising exposing immune cells to a viral vector encoding the gene of interest and a polypeptide having at least 70% sequence identity to one of SEQ ID NO:1, SEQ ID NO:2 or SEQ ID NO:3, under conditions suitable for transduction of the cells with said viral vector to generate a transduced cell for use in adoptive cell therapy.

In some embodiments, the polypeptide has at least 75% sequence identity to one of SEQ ID NO:3, SEQ ID NO:2, SEQ ID NO:6, SEQ ID NO:7, SEQ ID NO:8, SEQ ID NO:9, SEQ ID NO:10, SEQ ID NO:11, SEQ ID NO:12, SEQ ID NO:16, SEQ ID NO:17, SEQ ID NO:18, SEQ ID NO:19, SEQ ID NO:20, SEQ ID NO:21, SEQ ID NO:22, and/or SEQ ID NO:23, preferably wherein the polypeptide has one of at least 80%, 85%, 90%, 95%, or 99% sequence identity to one of SEQ ID NO:3, or SEQ ID NO:2, SEQ ID NO:6, SEQ ID NO:7, SEQ ID NO:8, SEQ ID NO:9, SEQ ID NO:10, SEQ ID NO:11, SEQ ID NO:12, SEQ ID NO:16, SEQ ID NO:17, SEQ ID NO:18, SEQ ID NO:19, SEQ ID NO:20, SEQ ID NO:21, SEQ ID NO:22, and/or SEQ ID NO:23.

In some embodiments, the polypeptide has at least 75% sequence identity to one of SEQ ID NO:3, SEQ ID NO:2, SEQ ID NO:6, SEQ ID NO:7, SEQ ID NO:8, SEQ ID NO:9, SEQ ID NO:10, SEQ ID NO:11, and/or SEQ ID NO:12, preferably wherein the polypeptide has one of at least 80%, 85%, 90%, 95%, or 99% sequence identity to one of SEQ ID NO:3, or SEQ ID NO:2, SEQ ID NO:6, SEQ ID NO:7, SEQ ID NO:8, SEQ ID NO:9, SEQ ID NO:10, SEQ ID NO:11, and/or SEQ ID NO:12.

In some embodiments, the polypeptide has at least 75% sequence identity to one of SEQ ID NO:3 and/or SEQ ID NO:2, preferably wherein the polypeptide has one of at least 80%, 85%, 90%, 95%, or 99% sequence identity to one of SEQ ID NO:2 and/or SEQ ID NO:3.

In some embodiments, the polypeptide comprises an amino acid sequence having at least 70% sequence identity to SEQ ID NO:6, preferably wherein the polypeptide comprises one of at least 80%, 85%, 90%, 95%, or 99% sequence identity to SEQ ID NO:6.

In some embodiments, the polypeptide comprises an amino acid sequence having at least 70% sequence identity to SEQ ID NO:7, preferably wherein the polypeptide comprises one of at least 80%, 85%, 90%, 95%, or 99% sequence identity to SEQ ID NO:7.

In some embodiments, the polypeptide comprises an amino acid sequence having at least 70% sequence identity to SEQ ID NO:8, preferably wherein the polypeptide comprises one of at least 80%, 85%, 90%, 95%, or 99% sequence identity to SEQ ID NO:8.

In some embodiments, the polypeptide comprises an amino acid sequence having at least 70% sequence identity to SEQ ID NO:9, preferably wherein the polypeptide comprises one of at least 80%, 85%, 90%, 95%, or 99% sequence identity to SEQ ID NO:9.

In some embodiments, the polypeptide comprises an amino acid sequence having at least 70% sequence identity to SEQ ID NO:10, preferably wherein the polypeptide comprises one of at least 80%, 85%, 90%, 95%, or 99% sequence identity to SEQ ID NO:10.

In some embodiments, the polypeptide comprises an amino acid sequence having at least 70% sequence identity to SEQ ID NO: 11, preferably wherein the polypeptide comprises one of at least 80%, 85%, 90%, 95%, or 99% sequence identity to SEQ ID NO:11.

In some embodiments, the polypeptide comprises an amino acid sequence having at least 70% sequence identity to SEQ ID NO:12, preferably wherein the polypeptide comprises one of at least 80%, 85%, 90%, 95%, or 99% sequence identity to SEQ ID NO:12.

In some embodiments, the polypeptide comprises an amino acid sequence having at least 70% sequence identity to SEQ ID NO:13, preferably wherein the polypeptide comprises one of at least 80%, 85%, 90%, 95%, or 99% sequence identity to SEQ ID NO:13.

In some embodiments, the polypeptide comprises an amino acid sequence having at least 70% sequence identity to SEQ ID NO:14, preferably wherein the polypeptide comprises one of at least 80%, 85%, 90%, 95%, or 99% sequence identity to SEQ ID NO:14.

In some embodiments, the polypeptide comprises an amino acid sequence having at least 70% sequence identity to SEQ ID NO:15, preferably wherein the polypeptide comprises one of at least 80%, 85%, 90%, 95%, or 99% sequence identity to SEQ ID NO:15.

In some embodiments, the polypeptide comprises an amino acid sequence having at least 70% sequence identity to SEQ ID NO:16, preferably wherein the polypeptide comprises one of at least 80%, 85%, 90%, 95%, or 99% sequence identity to SEQ ID NO:16.

In some embodiments, the polypeptide comprises an amino acid sequence having at least 70% sequence identity to SEQ ID NO:17, preferably wherein the polypeptide comprises one of at least 80%, 85%, 90%, 95%, or 99% sequence identity to SEQ ID NO:17.

In some embodiments, the polypeptide comprises an amino acid sequence having at least 70% sequence identity to SEQ ID NO:18, preferably wherein the polypeptide comprises one of at least 80%, 85%, 90%, 95%, or 99% sequence identity to SEQ ID NO:18.

In some embodiments, the polypeptide comprises an amino acid sequence having at least 70% sequence identity to SEQ ID NO:19, preferably wherein the polypeptide comprises one of at least 80%, 85%, 90%, 95%, or 99% sequence identity to SEQ ID NO:19.

In some embodiments, the polypeptide comprises an amino acid sequence having at least 70% sequence identity to SEQ ID NO:20, preferably wherein the polypeptide comprises one of at least 80%, 85%, 90%, 95%, or 99% sequence identity to SEQ ID NO:20.

In some embodiments, the polypeptide comprises an amino acid sequence having at least 70% sequence identity to SEQ ID NO:21, preferably wherein the polypeptide comprises one of at least 80%, 85%, 90%, 95%, or 99% sequence identity to SEQ ID NO:21.

In some embodiments, the polypeptide comprises an amino acid sequence having at least 70% sequence identity to SEQ ID NO:22, preferably wherein the polypeptide comprises one of at least 80%, 85%, 90%, 95%, or 99% sequence identity to SEQ ID NO:22.

In some embodiments, the polypeptide comprises an amino acid sequence having at least 70% sequence identity to SEQ ID NO:23, preferably wherein the polypeptide comprises one of at least 80%, 85%, 90%, 95%, or 99% sequence identity to SEQ ID NO:23.

In some embodiments, the polypeptide improves transduction efficiency compared to the transduction efficiency without the use of the polypeptide. In some embodiments, the polypeptide improves transduction efficiency by at least 1.1 times compared to transduction efficiency without the use of the polypeptide. In some embodiments, the polypeptide improves transduction efficiency by at least 1.2 times compared to the transduction efficiency without the use of the polypeptide, preferably wherein the polypeptide improves transduction efficiency by at least 1.3, 1.4, 1.5, 1.75, 2.0, 2.25, 2.5, 2.75, 3.0, 3.25, 3.5, 3.75, or 4.0 times compared to transduction efficiency without the use of the polypeptide.

In some embodiments, the polypeptide improves transduction efficiency compared to the transduction efficiency without the use of a transduction enhancer. In some embodiments, the polypeptide improves transduction efficiency by at least 1.1 times compared to transduction efficiency without the use of a transduction enhancer. In some embodiments, the polypeptide improves transduction efficiency by at least 1.2 times compared to the transduction efficiency without the use of a transduction enhancer, preferably wherein the polypeptide improves transduction efficiency by at least 1.3, 1.4, 1.5, 1.75, 2.0, 2.25, 2.5, 2.75, 3.0, 3.25, 3.5, 3.75, or 4.0 times compared to transduction efficiency without the use of a transduction enhancer.

In some embodiments, the polypeptide improves transduction efficiency compared to the transduction efficiency without the use of a transduction enhancer. In some embodiments, the polypeptide improves transduction efficiency by at least 1.5 times compared to transduction efficiency without the use of a transduction enhancer. In some embodiments, the polypeptide improves transduction efficiency by at least 1.75 times compared to the transduction efficiency without the use of a transduction enhancer, preferably wherein the polypeptide improves transduction efficiency by at least 2.0, 2.25, 2.5, 2.75, 3.0, 3.25, 3.5, 3.75, or 4.0 times compared to transduction efficiency without the use of a transduction enhancer.

In some embodiments, the polypeptide has at least 75% sequence identity to SEQ ID NO:3, SEQ ID NO:2, or SEQ ID NO:1, preferably wherein the polypeptide has at least 80%, 85%, 90%, 95%, or 99% sequence identity to SEQ ID NO:3, SEQ ID NO:2, or SEQ ID NO:1. In some embodiments, the polypeptide is vitronectin.

In some embodiments, the polypeptide has at least 75% sequence identity to SEQ ID NO:3, SEQ ID NO:2, or SEQ ID NO:1 or at least 75% sequence identity to a fragment of SEQ ID NO:3, SEQ ID NO:2, or SEQ ID NO:1, preferably wherein the polypeptide has at least 80%, 85%, 90%, 95%, or 99% sequence identity to SEQ ID NO:3, SEQ ID NO:2, or SEQ ID NO:1 or a fragment of SEQ ID NO:3, SEQ ID NO:2, or SEQ ID NO:1. In some embodiments, the polypeptide is vitronectin or a fragment of vitronectin.

In some embodiments, the immune cell is a mammalian immune cell. In some embodiments, the mammalian immune cell is a human immune cell. In some embodiments, the cell for use in adoptive cell therapy is a CAR expressing cell. In some embodiments, the CAR expressing immune cell is a CAR T-cell or CAR-Natural Killer (CAR-NK) cell.

In some embodiments, the method further comprises a step of harvesting one or more transduced cells.

In some embodiments, the method further comprises a step of expanding the transduced cells. In some embodiments, the method further comprises a step of expanding the transduced cells after they are harvested. In some embodiments, the method further comprises a step of storing the transduced cells. In some embodiments, the method further comprises a step of storing a CAR T-cell or CAR-Natural Killer (CAR-NK) cell.

In some embodiments, methods comprise the stimulation and/or expansion of immune cells. In some embodiments, methods comprise the stimulation and/or expansion of peripheral blood cells. In some embodiments, methods comprise the expansion of an immune cell population (e.g. a population of T-cells) from within a population of immune cells (e.g. PBMCs).

In some embodiments, a population of T-cells is expanded from within a population of tumor-infiltrating lymphocytes, by stimulation of the T-cells within the population of tumor-infiltrating lymphocytes.

In some embodiments, the method further comprises a step of formulating transduced mammalian cells with a pharmaceutically acceptable excipient to generate a medicament.

In some embodiments, the method further comprises expanding a population of T-cells from within a population of heterogeneous T-cells obtained from a blood sample, a population of PBMCs, or from a population of tumor-infiltrating lymphocytes.

In a further aspect, the present invention provides a CAR-expressing immune cell produced by a method as described above. In some embodiments, the CAR-expressing cell is a CAR-T cell or a CAR-NK cell, optionally mammalian or human.

In another aspect, the present invention provides a CAR-expressing immune cell produced by a method as described above for use in a method of treatment or prevention of a disease or disorder.

In another aspect, the present invention provides a method of treating or preventing a disease or disorder with a CAR-expressing immune cell produced by a method as described above.

In another aspect, the present invention provides the use of a CAR-expressing immune cell produced by a method as described above in the manufacture of a medicament for use in a method of treatment or prevention of a disease or disorder.

In some embodiments, the CAR-expressing immune cell is a CAR-T cell or a CAR-NK cell, optionally mammalian or human.

In some embodiments, the disease or disorder is cancer.

In some embodiments, the cancer is one or more of acute lymphocytic leukemia (ALL), acute myeloid leukemia (AML), adrenocortical cancer, anal cancer, bladder cancer, blood cancer, bone cancer, brain tumor, breast cancer, cancer of the female genital system, cancer of the male genital system, central nervous system lymphoma, cervical cancer, childhood rhabdomyosarcoma, childhood sarcoma, chronic lymphocytic leukemia (CLL), chronic myeloid leukemia (CML), colon and rectal cancer, colon cancer, endometrial cancer, endometrial sarcoma, esophageal cancer, eye cancer, gallbladder cancer, gastric cancer, gastrointestinal tract cancer, hairy cell leukemia, head and neck cancer, hepatocellular cancer, Hodgkin's disease, hypopharyngeal cancer, Kaposi's sarcoma, kidney cancer, laryngeal cancer, leukemia, leukemia, liver cancer, lung cancer, malignant fibrous histiocytoma, malignant thymoma, melanoma, mesothelioma, multiple myeloma, myeloma, nasal cavity and paranasal sinus cancer, nasopharyngeal cancer, nervous system cancer, neuroblastoma, non-Hodgkin's lymphoma, oral cavity cancer, oropharyngeal cancer, osteosarcoma, ovarian cancer, pancreatic cancer, parathyroid cancer, penile cancer, pharyngeal cancer, pituitary tumor, plasma cell neoplasm, primary CNS lymphoma, prostate cancer, rectal cancer, respiratory system, retinoblastoma, salivary gland cancer, skin cancer, small intestine cancer, soft tissue sarcoma, stomach cancer, stomach cancer, testicular cancer, thyroid cancer, urinary system cancer, uterine sarcoma, vaginal cancer, vascular system, Waldenstrom's macroglobulinemia and Wilms' tumor.

In some embodiments, the CAR is a CD19-CAR.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1. RetroNectin® promotes 293T cell adhesion in reduced serum media culture conditions.

Viral titre produced by HEK293T cells was assessed. No differences were seen in viral titre for HEK293T cells cultured in 10% FBS-supplemented DMEM in different growth conditions. However, in reduced 5% FBS-supplemented DMEM, RetroNectin® significantly promoted cell adhesion compared to non-treated, human platelet lysate (hPL), and FBS supplemented media. Data are presented throughout this study as means±standard error of the mean (S.E.M). Statistical significance was evaluated by Student's t-test and * p<0.05 was considered significant; ** p<0.01; *** p<0.001.

FIG. 2A-D. RetroNectin® can be repurposed to promote adherent viral vector production in different serum culture medias and is dose dependent. FIG. 2A shows microscopic images of cultures and demonstrates differences in cell adhesion, cell aggregation, and cell distribution. FIG. 2B shows a significant increase in 293T cell proliferation in RetroNectin® (8 μg/mL in PBS) coated 6-well plate over a period of 3 days in different types of serum free media as compared to the corresponding wells of similar culture media without any coating as controls (i.e., same volume of PBS). All cells were seeded in 300K/well, and the cells were counted using trypan blue dye exclusion assay. Data are presented as means±standard deviation of the mean. Statistical significance was evaluated by Student's t-test and * p<0.05 was considered significant; ** p<0.01. FIG. 2C shows viral titre measurements for cells grown in ImmunoCult™-XF T Cell Expansion Medium (StemCells Technologies Inc.) 4Cell Nutri-T Medium (abbreviated as NutriT; Sartorius AG), and TexMacs media (Miltenyi Biotech GmbH) in the absence and presence of RetroNectin®. FIG. 2D shows the effect of RetroNectin® concentration on viral vector titre. Data are presented throughout this study as means±standard error of the mean (S.E.M). Statistical significance was evaluated by Student's t-test and * p<0.05 was considered significant; ** p<0.01; *** p<0.001

FIG. 3A-C. Viral vector production using an adherent culture process and FBS-free media can be reproduced in different culture vessels. FIG. 3A shows viral vector titre for 9.5 cm³ cultures. FIG. 3B shows viral vector titre for 75 cm³ cultures. FIG. 3C shows viral vector titre for 175 cm³ cultures. Data are presented throughout this study as means±standard error of the mean (S.E.M).

FIG. 4A-B. Scale up and purification of FBS-free adherent viral vector. FIG. 4Ai shows the morphology of HEK293T cells seeded in 10% FBS-containing DMEM. FIG. 4Aii shows the morphology of HEK293T cells seeded in FBS-free NutriT media.

FIG. 5A-D. Vitronectin can be added as a solution to facilitate 293T cell adhesion under FBS-free conditions. FIG. 5A shows microscopic images of cultures and demonstrates differences in cell adhesion, cell aggregation, and cell distribution. FIG. 5B shows a significant increase in 293T cell proliferation in vitronectin (5 μg/mL in PBS) coated 6-well plate over a period of 3 days in different types of serum free media as compared to the corresponding wells of similar culture media without any coating as controls (i.e., same volume of PBS). All cells were seeded in 300K/well, and the cells were counted using trypan blue dye exclusion assay. Data are presented as means±standard deviation of the mean. Statistical significance was evaluated by Student's t-test and * p<0.05 was considered significant; ** p<0.01. FIG. 5C shows viral titre measurements for cells grown in ImmunoCult™-XF T Cell Expansion Medium (StemCells Technologies Inc.) 4Cell Nutri-T Medium (abbreviated as NutriT; Sartorius AG), and TexMacs media (Miltenyi Biotech GmbH) in the absence and presence of vitronectin. FIG. 5D shows the effect of vitronectin concentration on viral vector titre. Data are presented throughout this study as means±standard error of the mean (S.E.M). Statistical significance was evaluated by Student's t-test and * p<0.05 was considered significant; ** p<0.01; *** p<0.001

FIG. 6A-D. Vitronectin and RetroNectin® enhance transduction of PBMCs to express genes of interest. FIG. 6A shows 4Cell Nutri-T Medium (abbreviated as NutriT; Sartorius AG) is more superior at supporting T cell expansion when compared to ImmunoCult™-XF T Cell Expansion Medium (StemCells Technologies Inc.). The measurement was taken at day 7 using trypan blue dye exclusion assay. FIGS. 6B, 6C 6D shows the level of CD19 CAR+ transduced PBMCs in the absence of either virus (control 1), or transduction enhancer (control 2), or in the presence of either RetroNectin® or vitronectin in CD3+, CD4+, and CD8+ respectively.

FIG. 7A-D. Potency of lentiviral vectors encoding a gene of interest. FIG. 7A shows the cytotoxicity of CD19 expressing CAR-T-lymphocytes produced in different medias with different additives against Raji Cells (Burkitt Lymphoma). FIG. 7B shows the cytotoxicity of CD19 expressing CAR-T-lymphocytes produced in different medias with different additives against Nalm6 cells (Acute Lymphoblastic Leukemia). FIG. 7C shows the levels of interferon-gamma secretion in the Raji cell model. FIG. 7D shows the levels of interferon-gamma secretion in the Nalm6 cell model. Data are presented throughout this study as means±standard error of the mean (S.E.M).

FIG. 8. Schematic of a representative method of producing a cell to be used in adoptive cell therapy.

FIG. 9A-B. The effect of long-term coating. FIG. 9A shows that 293T cells formed a monolayer on a plate surface within 2 hours post cell seeding after plate was subjected to long-term RetroNectin® coating, whereas control cells (293T cells grown in either 10% FBS supplemented DMEM or NutriT media alone) displayed significant aggregation at the same time-point. FIG. 9B shows that virus production was just as efficient in cells seeded for just 3 hours in wells with long term RetroNectin® coating as cells that were seeded 72 h before transfection using standard techniques. Data are presented throughout this study as means±standard error of the mean (S.E.M).

FIGS. 10A-10C. Design of polypeptides derived from vitronectin and RetroNectin®. FIG. 10A shows schematic showing the structures of recombinant RV #1 and RV #2 proteins, as well as the structure of vitronectin. Locations within vitronectin of peptides from FIG. 1C are indicated. FIG. 10B shows sequences of recombinant RV #1 and RV #2 proteins. FIG. 10C shows sequences of polypeptides derived from vitronectin (P1-7) or RetroNectin® (PR1, PR7.1 and PR7.2).

FIGS. 11A-11C. Impact of polypeptides derived from vitronectin and RetroNectin® on transduction efficiency. FIG. 11A shows a bar chart showing comparison of the percentage of CD19 CAR+ cells from cultures transduced in the presence of RetroNectin®, vitronectin and predicted transduction enhancer proteins and peptides. RetroNectin® was used as the standard transduction enhancer control. FIG. 11B shows a bar chart showing comparison of the percentage of CD19 CAR+ cells from cultures transduced in the presence of increasing concentrations of vitronectin. FIG. 11C shows a bar chart showing comparison of the percentage of CD19 CAR+ cells from cultures transduced in the presence of increasing concentrations of peptide P1.

FIGS. 12A-12I. Impact of transduction enhancers on IL-6 secretion following transduction. FIG. 12A shows the concentration of IL-6 secreted by transduced T cells after 4-days expansion following transduction with hCD19-CAR in the presence of RetroNectin®, vitronectin, or in the absence of any transduction enhancer. FIG. 12B shows the concentration of IL-6 secreted by transduced T cells after 14 days expansion following transduction with hCD19-CAR in the presence of RetroNectin®, vitronectin, or in the absence of any transduction enhancer. FIG. 12C shows the concentration of IL-6 secreted by hCD19-CAR-transduced T cells transduced with increasing amounts of vitronectin. FIG. 12D shows the percentage of specific cell killing mediated by hCD19-CAR-transduced T cells when transduced in the presence of RetroNectin®, vitronectin, or without added reagents. FIG. 12E shows the detection of vitronectin in transduced CAR T cell cultures—vitronectin was detected (~20 ng/mL) in Day 4 harvested CAR T cells, but no vitronectin was detected at Day 14. FIG. 12F shows the concentration of TNF alpha secreted by transduced T cells after 4 days of expansion following transduction. FIG. 12G shows the concentration of IFN gamma secreted by transduced T cells after 4 days of expansion following transduction. FIG. 12H shows the concentration of IL-8 secreted by transduced T cells after 14 days of expansion following transduction. FIG. 121 shows the level of granzymes at a 5:1 Effector to Target ratio.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

During the course of optimization of the manufacturing process of lentiviral vectors, inventors found that lentiviral vectors are very stable in an FBS-free T cell media known as TexMacs media. Inventors then considered whether it might be possible to use serum-free/FBS-free media that supports both T cell proliferation and cell expansion for LV production. Adopting a common media to support key processes from LV production to the modification of T cells with LV expressing chimeric antigen receptors (CARs) could improve the consistency of the final T cell products which would otherwise be exposed to multiple media formulations.

The present invention has been devised in light of the above considerations.

Inventors developed a novel serum-free adherent process (NSAP) for viral vector production. This method is made possible through identification, characterization, and re-purposing of GMP-compliant materials (vitronectin and RetroNectin®).

### Methods of Culturing Cells

Any suitable method of culturing cells, for example as set out in the Examples, may be used in the methods and compositions described here.

Cell culture generally refers to the removal of cells from an organism and their subsequent growth in a favourable artificial environment. The cells may be removed from the tissue directly and disaggregated by enzymatic or mechanical means before cultivation, or they may be derived from a cell line or cell strain that has already been established. They can subsequently be maintained under carefully controlled conditions.

In some embodiments, the methods described herein relate to the culture of cell lines. In some embodiments, these cell lines are already known. In some embodiments these cell lines are insect or mammalian cell lines. Preferably the mammalian cell lines are human cell lines. The human cell lines may be human embryonic kidney cell, HeLa cell, or HT-1080 cell lines. In some embodiments the human embryonic kidney cell lines are HEK293 cell, HEK293T cell, or HEK293FT cell lines.

Cells can be grown under a range of different conditions. However, generally, there are conditions which lead to improved growth rates and yields of cells. These conditions vary for each cell type, but generally consist of providing a suitable culture vessel with a substrate or medium that supplies the essential nutrients (e.g. amino acids, carbohydrates, vitamins, and/or minerals), growth factors, hormones, and/or gases (CO₂, O₂) for cells to grow.

Many cells require a surface or an artificial substrate to form an adherent culture as a monolayer, whereas other cells can be grown free floating in a medium as a suspension culture.

### Adherent Cell Culture

At present, 70% of viral vector products are manufactured using adherent culture processes where cells are grown as monolayers in culture vessels instead of free-floating in culture media (i.e., suspension). Adherent-based manufacturing processes can be readily procured off-the-shelf and require less expert bioengineering know-how compared to three-dimensional platforms. However, most adherent processes are dependent on products of animal origin such as fetal bovine serum (FBS) that pose safety, consistency and regulatory challenges.

Aspects of the present invention provide methods of culturing adherent cells comprising culturing the cells in a cell culture vessel. Methods of culturing cells is also referred to herein as a method of expanding a cell population, since a cultured cell population will be expanded by the method. The cell cultures are not suspension cultures as the cells are adhered to a/the surface of the culture vessel and/or a coating on a/the surface of the culture vessel.

Such methods of the invention may comprise seeding the cells at the liquid-liquid interface of the cell culture vessel and contacting the seeded cells with a cell culture medium. The choice of cell culture medium will be particular to the cells being cultured and the skilled person is familiar with which cell culture media are suitable for which cell types. In some embodiments of the invention, the cells are cultured for at least about 1 day, or at least about 5 days, or at least about 7 days, or at least about 14 days. In preferred embodiments, the cells are cultured for at least about 7 days. In some embodiments of the invention, the cells are cultured for less than about 5 days, about 7 days, about 14 days, about 28 days, about 56 days, or about 100 days. In preferred embodiments, the cells are cultured for at least about 7 days. Advantageously, the cell cultures systems of the present invention are suitable for this long-term culture of adherent cells.

By “adherent culture” or “culturing under adherent conditions” it is meant culturing under conditions that promote the adhesion of cells to the container in which they are cultured, e.g. the bottom of a tissue culture plate or flask (Jaccard et al. JALA Charlottesv Va. (2014) 19(5):437-443). In some instances, cells may be induced to adhere to the container simply by keeping the culture stationary.

The level of adhesion of cells to a surface such as a culture vessel can be determined in a number of ways. Firstly, the level of cell adhesion can be assessed through microscopy to visually assess the level of adhesion to a given surface. The level of cell adhesion can also be assessed through a flow chamber pressure assay in which cultured cells are subjected to pressure through a flow chamber in a microfluidic setup, optionally wherein the microfluidic setup is attached to a microscope and/or a camera to assess the level of adhesion. A non-invasive and label-free real-time impedance assay can be employed using electronic plates to record cellular vigour and viability at temporal resolution to determine the level of cell adhesion. Additionally, cells could be stained with anti-cell adhesion antibodies to determine the number of cells which are adhered compared to cells which have not adhered. Furthermore, the level of cell aggregation is a strong indicator of the level of cell adhesion i.e. when cellular aggregation is low then cell adhesion is high. The skilled person would be aware of additional methods which could be used to determine the level of cell adhesion.

### Cells for Adherent Cell Culture

Aspects of the present invention provide methods of culturing adherent cells comprising culturing the cells in a cell culture vessel.

Adherent cells can grow and expand whilst attached to a surface such as a surface of a culture vessel. In some cases, adherent cells grow and expand as a monolayer on a surface such as a surface of a culture vessel. Based on their morphology, adherent cells can be divided into two main categories: Fibroblast-like cells which are elongated in shape and usually migrate on the dish, or epithelial-like cells which are generally polygonal in shape and are generally stationary and grow in patches.

Adherent cells are often used to produce viral vectors. As mentioned previously, 70% of viral vector products are manufactured using adherent culture processes with adherent cells. The majority of the cells derived from vertebrates and insects are anchorage-dependent and can be cultured on a suitable substrate. Therefore, examples of adherent cells include insect cells and mammalian cells.

Mammalian cells are commonly used throughout biotechnology. Mammalian cells are excellent hosts for the production of recombinant proteins, viral vectors, and other products of interest. Additionally, mammalian cells themselves can be used in therapies, for example in adoptive cell transfer and stem cell therapeutics. Human cells are often used in human therapeutics. Mammalian cells, such as human cells, can be grown via adherent cell culture. Human cells grown in adherent culture may, for example, be human embryonic kidney cells, HeLa cells, or HT-1080 cells.

Mammalian cells may be derived from, any mammalian animal or human, e.g. non-human animals, e.g. rabbit, guinea pig, rat, mouse or other rodent (including from any animal in the order Rodentia), cat, dog, pig, sheep, goat, cattle (including cows, e.g. dairy cows, or any animal in the order Bos), horse (including any animal in the order Equidae), donkey, and non-human primate or other non-human vertebrate organism; and/or non-human mammalian animal; and/or human.

Human embryonic kidney (HEK) cells have been widely used in cell biology research for many years, because of their reliable growth and propensity for transfection. Examples of HEK cell lines are HEK293 cells, or cell lines derived from the HEK 293 cell line such as HEK293T, HEK293FT, HEK293S, HEK29FTM, HEK293SG, HEK293SGGD, HEK293H, HEK293E, HEKEBNA1-6E, HEK293MSR and HEK293A cells. HEK293 cells were generated in 1973 by transfection of cultures of normal human embryonic kidney cells with sheared adenovirus 5 DNA in Alex van der Eb's laboratory in Leiden, the Netherlands (Graham et al. J Gen Virol. (1977) 36(1):59-74). HEK 293 cells are straightforward to grow in culture and to transfect.

An important variant of the HEK293 cell line is the HEK293T cell line. It contains the SV40 large T-antigen that allows for episomal replication of transfected plasmids containing the SV40 origin of replication. This allows for amplification of transfected plasmids and extended temporal expression of desired gene products. HEK293 and HEK293T cells are commonly used for the production of various viral vectors.

HeLa cells are an immortal cell line used in scientific research. It is the oldest and most commonly used human cell line. The line is named after and derived from cervical cancer cells taken on Feb. 8, 1951, from Henrietta Lacks. HeLa cells can be modified to express viral vectors.

HT-1080 is a fibrosarcoma cell line which has been used extensively in biomedical research. The cell line was created from tissue taken in a biopsy of a fibrosarcoma present in a 35-year-old human male. HT-1080 cells can be modified to express viral vectors.

Insect cells can efficiently express recombinant proteins and can be used for the development of viral vectors, virus-like particles and vaccines. Insect cell lines can be derived from the Orders Lepidoptera (moths and butterflies) and Diptera (flies). Insect cells can be derived from Bombyx mori, Mamestra brassicae, Spodoptera frugiperda, Trichoplusia ni, and Drosophila melanogaster. Examples of insect cell lines are: Sf9, IPLB-Sf21AE (Sf21), Sf900+, Schneider 2 (S2), TN-368, BTI-TN-5B1-4 (High Five), Tni PRO, and Bm-N. Insect cells can be modified to express viral vectors.

### Types of Culture Media

A growth medium or culture medium is a liquid, semi-solid or solid designed to support the growth and expansion of cells. Culture media are well known by scientists in the area of cell culture. Cell culture media types and methods are comprehensively reviewed in “Cell Culture Media: A Review” (Meenakshi Arora, 2013. MATER METHODS 3:175), the contents of which are incorporated by reference.

Culture media generally comprise an appropriate source of energy and compounds which regulate the cell cycle. A typical culture medium is composed of a complement of amino acids, vitamins, inorganic salts, glucose, and serum as a source of growth factors, hormones, and attachment factors. In addition to nutrients, the medium also helps maintain pH and osmolality.

Culture media may be liquid (i.e. aqueous), semi-solid (i.e. gelatinous), or completely solid. Semi-solid and solid media may contain agar, silica gel, acrylamide, gellan gum, or other solidification agents. Liquid media generally do not contain solidification agents.

Culture media may be natural media, e.g. biological fluids, tissue extracts, or clots. Additionally, culture media may be artificial media such as balanced salt solutions (e.g. PBS, DPBS, HBSS, EBSS), basal media (e.g. MEM, and DMEM), or complex media (e.g. RPMI-1640, and IMDM).

Natural media consist solely of naturally occurring biological fluids. Natural media are very useful and convenient for a wide range of animal cell culture. The major disadvantage of natural media is its poor reproducibility due to lack of knowledge of the exact composition of these natural media. Natural media is generally not serum-free.

Artificial or synthetic media are prepared by adding nutrients (both organic and inorganic), vitamins, salts, O₂ and CO₂ gas phases, serum proteins, carbohydrates, cofactors. Different artificial media have been devised to serve one or more of the following purposes: 1) immediate survival (a balanced salt solution, with specific pH and osmotic pressure); 2) prolonged survival (a balanced salt solution supplemented with various formulation of organic compounds and/or serum); 3) indefinite growth; 4) specialized functions.

Artificial media are often grouped into four categories: serum containing media, serum-free media, chemically defined media, and protein-free media.

Serum containing media often contains FBS. FBS is the most common supplement in animal cell culture media. It is used as a low-cost supplement to provide an optimal culture medium. Serum binds and neutralizes toxic moieties, and provides carriers or chelators for labile or water-insoluble nutrients, hormones and growth factors, and protease inhibitors.

Serum-free media can be formulated to support the culture of a single cell type, such as Knockout Serum Replacement and Knockout DMEM from Thermo Fisher Scientific, and mTESR medium from Stem Cell Technologies, for stem cells. Serum-free media incorporate defined quantities of purified growth factors, lipoproteins, and other proteins, which are otherwise usually provided by the serum. These media are also referred to as ‘defined culture media’ since the components in these media are known.

Chemically defined media contain contamination-free ultra-pure inorganic and organic ingredients, and may also contain pure protein additives, like growth factors. Their constituents are produced in bacteria or yeast by genetic engineering with the addition of vitamins, cholesterol, specific amino acids, and fatty acids.

Protein-free media do not contain any protein and only contain non-protein constituents. Compared to serum-supplemented media, use of protein-free media promotes superior cell growth and protein expression and facilitates downstream purification of any expressed product. Formulations like MEM, RPMI-1640 are protein-free and protein supplement is provided when required.

The methods and compositions described here may include culture of cells in a fetal bovine serum (FBS)-free medium. The term “FBS-free media” or “FBS-free medium” may comprise cell culture media in which the presence of fetal bovine serum (FBS) cannot be identified and/or detected.

FBS-free media are commercially available as, for example, ImmunoCult™-XF T Cell Expansion Medium (StemCells Technologies Inc.), ImmunoCult™-XF B Cell Expansion Medium (StemCells Technologies Inc.), ImmunoCult™ NK Cell Expansion Medium (StemCells Technologies Inc.), ClonaCell™-CHO ACF Medium (StemCells Technologies Inc.), ImmunoCult™-SF Macrophage Medium (StemCells Technologies Inc.), 4Cell Nutri-T Medium (Sartorius AG), BIOTARGET™, DCCM-1 SFM (Sartorius AG), BIO-MPM-1 SFM (Sartorius AG), MSCgo™ Osteogenic Differentiation Medium (Sartorius AG), MSCgo™ Rapid Osteogenic Differentiation Medium (Sartorius AG), NutriStem® hPSC XF Medium (Sartorius AG), MSC NutriStem® XF Medium (Sartorius AG), EndoGo™ XF Medium (Sartorius AG), MSCgo™ Adipogenic Differentiation Medium (Sartorius AG), NutriFreez® D10 Cryopreservation Medium (Sartorius AG), NutriFreez® D10 Cryopreservation Medium without Phenol Red (Sartorius AG), TexMacs media (Miltenyi Biotech GmbH), Gibco-BRL (Invitrogen), Virus Production (VP) Media from Gibco/ThermoFisher (Catalog number A4817902), OptiPRO SFM from Gibco/ThermoFisher (Catalog number 12309019), OpTmizer T cell expansion SFM from Gibco/ThermoFisher (Catalogue number A1048501), LymphoONE Media from Takara (Catalogue number is WK552s), X-Vivo media from Lonza (Catalogue number is 04-744Q), Prime-XV media from T cell CDM Irvine scientific (catalogue number is 91154), and AIM-V serum free media from Gibco/ThermoFisher (Catalogue number 12055083). Many more would be known to the skilled person.

Serum is the amber-coloured blood fraction remaining after the natural coagulation of blood; it is typically further refined via centrifugation, which serves to remove remaining blood cells. FBS is the serum derived from blood drawn from a bovine fetus via a closed system of collection. FBS is used as a growth supplement for the in vitro cell culture of eukaryotic cells. Animal sera (both bovine and non-bovine sera) are used in cell culture applications with the most widely used being fetal bovine serum (FBS).

Serum is a complex mix of albumins, growth factors and growth inhibitors. Serum was traditionally one of the most important components of cell culture media and serves as a source for amino acids, proteins, vitamins (particularly fat-soluble vitamins such as A, D, E, and K), carbohydrates, lipids, hormones, growth factors, minerals, and trace elements. Serum from fetal and calf bovine sources are commonly used to support the growth of cells in culture (von Seefried and Macmorine, 1976. Dev Biol Stand. 37:83-9). Fetal serum is a rich source of growth factors and is appropriate for cell cloning and for the growth of fastidious cells. Many growth media often contain 2-10% of serum.

Most adherent cell culture processes are dependent on products of animal origin like FBS that pose safety, consistency and regulatory challenges.

The methods and compositions described here may include culture of cells in a serum-free medium, defined culture media or chemically defined media. However, in some embodiments, the media may be FBS-free, but may contain other serum components.

The term “serum-free media” may comprise cell culture media which is free of serum proteins, e.g., FBS. Serum-free media are known in the art and are described for example in U.S. Pat. Nos. 5,631,159 and 5,661,034. Serum-free media are commercially available as, for example, ImmunoCult™-XF T Cell Expansion Medium (StemCells Technologies Inc.), ImmunoCult™-XF B Cell Expansion Medium (StemCells Technologies Inc.), ImmunoCult™ NK Cell Expansion Medium (StemCells Technologies Inc.), ClonaCell™-CHO ACF Medium (StemCells Technologies Inc.), ImmunoCult™-SF Macrophage Medium (StemCells Technologies Inc.), 4Cell Nutri-T Medium (Sartorius AG), BIOTARGET™, DCCM-1 SFM (Sartorius AG), BIO-MPM-1 SFM (Sartorius AG), MSCgo™ Osteogenic Differentiation Medium (Sartorius AG), MSCgo™ Rapid Osteogenic Differentiation Medium (Sartorius AG), NutriStem® hPSC XF Medium (Sartorius AG), MSC NutriStem® XF Medium (Sartorius AG), EndoGo™ XF Medium (Sartorius AG), MSCgo™ Adipogenic Differentiation Medium (Sartorius AG), NutriFreez® D10 Cryopreservation Medium (Sartorius AG), NutriFreez® D10 Cryopreservation Medium without Phenol Red (Sartorius AG), TexMacs media (Miltenyi Biotech GmbH), and Gibco-BRL (Invitrogen). Many more would be known to the skilled person.

The serum-free media may be supplemented with one or more components, such as serum replacement media, at a concentration of for example, 5%, 10%, 15%, etc. The serum-free media may be supplemented with 10% serum replacement media from Invitrogen-Gibco (Grand Island, New York).

The methods and compositions described here may include culture of cells in a xeno-free medium. However, in some embodiments, the media may be FBS-free, but may not be xeno-free.

The term “xeno-free medium” may comprise cell culture media that do not contain components derived from an organism other than the organism from which the cultured cells are derived. Therefore, when growing human cells, such as HEK cells, “xeno-free medium” refers to a formulation only comprised of human-derived components and does not incorporate foreign species such as bovine (e.g. FBS).

The methods and compositions described here may include culture of cells in an animal-product-free medium. However, in some embodiments, the media may be FBS-free, but may contain other animal-product components.

The term “animal-product-free medium” is defined in this specification as a medium which does not contain components derived from a non-human animal. An “animal-product-free medium” may contain a recombinant polypeptide that has been designed based on a non-human animal polypeptide which is not the same as the related non-human animal polypeptide.

The methods and compositions described here may include culture of cells in a protein-free medium. However, in some embodiments, the media may be FBS-free, but may contain other proteins.

The term “protein-free medium” may comprise cell culture media which does not contain any protein components. Proteins are well known by the skilled person. Proteins are large biomolecules and macromolecules that comprise one or more long chains of amino acid residues. RetroNectin®, vitronectin, polypeptides derived from RetroNectin® and/or polypeptides derived from vitronectin may be added as a supplement to a protein-free medium.

### Culture Vessels

Any suitable container or culture vessel may be used to propagate cells according to the methods and compositions described here. In some aspects, the container or culture vessel should comprise a surface on which adherent cells can attach or adhere to assist their growth.

The skilled person is aware of many different types of culture vessels. Exemplary culture vessels include: plates, dishes, flasks, bottles, cell factories and bioreactors. Also known as culture plates, culture dishes, culture flasks, culture bottles, culture cell factories or culture bioreactors. Culture vessels can be constructed from a number of materials such as metal, glass, plastics and polymers such as polystyrene and polyester.

Culture plates are low flat-bottomed laboratory containers for growing cells on a thin layer of nutrient medium. Plates and dishes can be used with liquid and solid media. Culture dishes are similar to culture plates. The most common types of culture plates and dishes are the Petri dish and the multiwell plate.

Multiwell plates are available in many sizes, for example as a 2-well plate, 4-well plate, a 6-well plate, a 12-well plate, a 24-well plate, a 48-well plate, a 96-well plate, or a 384 well plate. Petri dishes and multiwell plates are available from many manufacturers (e.g. Thermo Fisher Scientific) with different specifications. Plates are available in different colours (e.g. clear, black and white), and are usually made from polystyrene.

Culture flasks are generally used with liquid cultures and typically resemble bottles. Flasks are generally constructed from glass or polystyrene. It is possible to reuse and sterilise glass culture flasks, whereas disposable polystyrene flasks are widely available (e.g. Thermo Fisher Scientific Nunc EasYFlasks).

Culture flasks are available in many sizes, such as T25, T75, T175, T225, and T300. Different size flasks have different volumes and a different surface area for adherent cells to grow.

In some embodiments, the culture vessel has a volume of at least 0.5, 0.6, 0.7, 0.8, 0.9, 1.5, 1.0, 2.0, 3.0, 4.0, 5.0, 10.0, 15.0, 20.0, 25.0, 30.0, 35.0, 40.0, 45.0, 50.0, 60.0, 70.0, 80.0, 90.0, 100.0, 125.0, 150.0, 175.0, or 200.0 mL.

Cell factories, or cell factory systems, are multilayer systems for growing cells. Cell factories may comprise multiple layers of culture vessels (e.g. multiple layers of plates, multiple layers of flasks, or multiple layers of trays). Cell factories are available are available in many sizes with many different numbers of layers being possible. The skilled person would be aware of different types of cell factories which are widely available (e.g. Thermo Fisher Scientific Nunc EasyFill Cell Factory systems, or Corning High Yielding Performance Flask (HYPERFlask)).

A bioreactor may comprise a regulated bioreactor, in which one or more conditions may be controlled or monitored, for example, oxygen partial pressure. Devices for measuring and regulating these conditions are known in the art. For example, oxygen electrodes may be used for measuring oxygen partial pressure. The oxygen partial pressure can be regulated via the amount and the composition of the selected gas mixture (e.g., air or a mixture of air and/or oxygen and/or nitrogen and/or carbon dioxide). Suitable devices for measuring and regulating the oxygen partial pressure are described by Bailey, J E. (Bailey, J E., Biochemical Engineering Fundamentals, second edition, McGraw-Hill, Inc. ISBN 0-07-003212-2 Higher Education, (1986) or Jackson A T. Jackson A T., Verfahrenstechnik in der Biotechnologie, Springer, ISBN 3540561900 (1993)).

Other suitable containers include spinners. Spinners are a type of bioreactor, which can be agitated using various agitator mechanisms, such as glass ball agitators, impeller agitators, and other suitable agitators. The cultivation volume of a spinner is typically between 20 mL and 500 mL. Roller bottles are round cell culture flasks made of plastic or glass having a culture area of between 400 and 2000 cm². The cells are cultivated along the entire inner surface of these flasks; the cells are coated with culture medium accomplished by a “rolling” motion, i.e. rotating the bottles about their own individual axis.

Alternatively, culture may be static, i.e. where active agitation of the culture/culture media is not employed. By reducing agitation of the culture aggregates of cells/microcarriers may be allowed to form. Whilst some agitation may be employed to encourage distribution and flow of the culture media over the cultured cells this may be applied so as not to substantially disrupt aggregate formation. For example, a low rpm agitation, e.g. less than 30 rpm or less than 20 rpm, may be employed.

In some embodiments, culture vessels are coated with RetroNectin®, vitronectin, polypeptides derived from RetroNectin®, and/or polypeptides derived from vitronectin e.g. one or more surfaces of the culture vessel may be coated with RetroNectin®, vitronectin, polypeptides derived from RetroNectin®, and/or polypeptides derived from vitronectin which may optionally be combined with other nutrients or with the culture media. One or several layers of a coating may be applied to a culture vessel. The coating may form a substrate for cell growth and may increase adherence of cells to the culture vessel.

The culture vessel may be coated with a matrix, which in the context of this document refers to a layer (e.g. a thin layer or film) of substance attached to the culture vessel such as on its surface. The matrix may comprise RetroNectin, vitronectin, polypeptides derived from RetroNectin®, and/or polypeptides derived from vitronectin. The matrix may comprise a further compatible or physiologically relevant matrix capable of supporting growth of cells.

### Viral Vectors

Viral vectors are well known in the art, and are reviewed e.g. by Lundstrom, 2018. Diseases 6(2):42 and Morgan and Boyerinas, 2016. Biomedicines 4(2):9, both of which are hereby incorporated by reference in their entirety.

Viral vectors generally use the genome of a virus as a carrier for the nucleic acid of interest. That is, the nucleic acid of interest is inserted into the viral genome, and thus when the viral genome is introduced into the target cell, the nucleic acid of interest is also introduced.

In some embodiments the genome of the viral vector of the present invention is derived from the genome of an adenovirus (e.g. Ad5), an adeno-associated virus (e.g. AAV2, 3, 5, 6, 8 or 9), a herpes simplex virus (e.g. HSV, HSV1), a retrovirus (e.g. MMSV, MSCV), a lentivirus (e.g. SIV, HIV-1, HIV-2), an alphavirus (e.g. SFV, SIN, VEE, M1), a flavivirus (e.g. Kunjin, West Nile, Dengue), a rhabdovirus (e.g. rabies, VSV), a measles virus, (e.g. MV-Edm), Newcastle disease virus, a poxvirus (e.g. VV) or a picornavirus (e.g. Coxsackievirus).

In some embodiments, the genome of the viral vector is derived from the genome of a retrovirus (i.e. the viral vector is a retroviral vector). In some embodiments, the genome of the viral vector is derived from the genome of a lentivirus (i.e. the viral vector is a lentiviral vector). In some embodiments, the genome of the viral vector is derived from the genome of HIV. In some embodiments, the genome of the viral vector is derived from the genome of HIV-1.

The ability of a viral vector to deliver the nucleic acid of interest to a cell is determined by its ability to enter the cell, which is in turn determined by the ability of the viral vector to bind to and be internalised by the cell.

Viral vectors often use envelope glycoproteins capable of binding to, and mediating entry into, a wide range of cell types.

Lentiviruses are a subclass of Retroviruses. Lentiviruses comprise an envelope, a capsid and, an RNA genome. Lentiviruses enter in their target cells with a very high efficiency, and once inside the cell, RNA genome is reverse-transcribed in DNA and is integrated into the genome of the cell by a viral integrase enzyme. This wild-type biology can be modified to enable lentiviruses to work as an efficient gene transfer tool. To this end, the genes required for viral replication and inducing pathogenic effects are replaced by genes of interest, for example CAR genes.

The genomes of lentiviruses are favoured for preparing viral vectors e.g. because of their ability to introduce nucleic acid into target cells with high efficiency and stability. However, the envelope glycoproteins of lentiviruses bind to receptors which are only expressed by a small number of cell types. Viral vectors therefore often employ a lentiviral genome with the envelope glycoprotein of another virus, which is referred to as pseudotyping. Pseudotyped lentiviral vectors are reviewed e.g. by Joglekar and Sandoval, Human Gene Therapy Methods (2017) 28(6):291-301, which is hereby incorporated by reference in its entirety.

Lentiviral vectors, derived from the human immunodeficiency virus, have been extensively investigated and optimized over the past two decades. Third-generation, self-inactivating lentiviral vectors have recently been used in multiple clinical trials to introduce genes into hematopoietic stem cells to correct primary immunodeficiencies and hemoglobinopathies. These vectors have also been used to introduce genes into mature T cells to generate immunity to cancer through the delivery of chimeric antigen receptors (CARs) or cloned T-cell receptors (Milone and O'Doherty, Leukemia volume 32, pages 1529-1541. 2018)

First-generation lentiviral vectors contained a significant portion of the HIV genome, including the gag and pol genes, as well as several additional viral proteins (Vannucci L, et al. New Microbiol. (2013) 36:1-22). The envelope protein of another virus, most commonly VSV-G, was included in the design of first-generation lentiviral vectors. VSV-G recognizes a ubiquitously expressed receptor that was most recently identified as the low-density lipoprotein (LDL) receptor, allowing the lentiviral vector to transduce a wide range of cells (Burns et al. Proc Natl Acad Sci USA. (1993) 90:8033-7). The VSV-G gene was encoded on a separate plasmid from the other lentiviral genes.

Safer, second-generation lentiviral vectors lacking the accessory virulence factors vif, vpr, vpu, and nef were subsequently developed. Third-generation lentiviral vectors further improved safety by splitting the viral genome into separate plasmids, making recombinant virus generation even more unlikely.

Examples of envelope glycoproteins commonly used in viral vectors include e.g. vesicular stomatitis virus glycoprotein (VSV-G), Ebola glycoprotein, Mokola glycoprotein, LCMV glycoprotein and MuLV glycoprotein. VSV-G is commonly employed in viral vectors as its cognate LDL receptor is ubiquitously expressed, and so viral vectors having viral envelope glycoproteins including VSV-G are able to infect a wide range of cell types.

In some embodiments, the envelope protein is a vesicular stomatitis virus G (VSV-G) protein, a Baboon protein (e.g. BaEV), a RD114 protein, a measles virus protein (e.g. H/F), a cocal virus protein (e.g. COCV), a nipah virus protein, an Ebola protein, a Mokola protein, a LCMV protein, or a MuLV glycoprotein.

In some embodiments a viral envelope glycoprotein in accordance with the present invention is, or is derived from, VSV-G, Ebola glycoprotein, Mokola glycoprotein, LCMV glycoprotein, baboon glycoprotein, RD114 glycoprotein, measles glycoprotein, cocal glycoprotein, nipah glycoprotein, or MuLV glycoprotein. In some embodiments a viral envelope glycoprotein is, or is derived from, VSV-G.

As used herein, a viral envelope glycoprotein which is “derived from” a reference viral envelope glycoprotein may have high sequence identity to the amino acid sequence of the viral envelope glycoprotein from which it is derived. For example, a viral envelope glycoprotein which is “derived from” a reference viral envelope glycoprotein may have at least 60%, e.g. one of at least 65%, 70%, 75%, 80%, 85% 86%, 87%, 88%, 89%, 90%, 91%, 92%, 93%, 94%, 95%, 96%, 97%, 98%, 99%, or 100% sequence identity to the amino acid sequence of the viral envelope glycoprotein from which it is derived.

In some embodiments, the viral envelope glycoprotein comprises, or consists of, an amino acid sequence having at least 60%, e.g. one of at least 65%, 70%, 75%, 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:1.

### Methods of Producing Viruses, Viral Vectors and Viral Particles

Methods for producing viruses, viral vectors and viral particles are well known in the art, and are reviewed e.g. in Merten et al. Pharm. Bioprocess (2014) 2:183-203 and van der Loo and Wright, Human Molecular Genetics (2015):1-11, both of which are hereby incorporated by reference in their entirety.

The methods generally involve introducing nucleic acid encoding the components of the viral vector into cells e.g. by transfection or infection of the cells.

The most commonly used method for generating retroviral, lentiviral and adeno-associated viruses, viral vectors and viral particles involves co-transfection of HEK 293 cells with plasmids encoding the nucleic acid of interest to be transferred, packaging components encoding the structural proteins required for virion assembly, and the envelope protein.

Co-transfected cells are cultured under conditions suitable for expression of the nucleic acid of interest and viral proteins from the plasmids, and the components of the viral vector assemble into virions, which are then released by the cells in culture into the cell culture supernatant. Cell culture supernatant containing the viral particles can be isolated and/or collected, optionally processed to remove cells and cell debris, and optionally concentrated. Concentration may comprise centrifugal filtration, or precipitation of viral particles using calcium phosphate (see e.g. Pham et al. J Gene Med. (2001) 3(2):188-94).

The plasmid encoding the nucleic acid of interest typically also comprises a promoter for driving expression of the nucleic acid of interest. In lentiviral systems the nucleic acid of interest is generally flanked by viral long terminal repeat (LTR) sequences in order to facilitate integration of the nucleic acid of interest into the target cell genome, and second generation lentiviral systems utilise a viral promoter in the 5′ LTR to drive expression of the nucleic acid of interest in a tat-dependent manner. Third generation lentiviral systems instead use a chimeric 5′ LTR comprising a promoter which is not tat-dependent, such as a CMV or RSV promoter. The LTR provided 3′ to the nucleic acid of interest may contain deletion rendering the virus self-inactivating after integration. Transfer plasmids may comprise additional promoters for driving expression of the nucleic acid of interest, and may comprise other elements such as central a polypurine tract (cPPT) for facilitating expression of the nucleic acid of interest, Psi for facilitating packaging by nucleocapsid and Woodchuck hepatitis virus post-transcriptional regulatory element (WPRE) which enhances expression of the nucleic acid of interest by increasing nuclear export.

The packaging plasmid(s) provide the genes encoding the viral proteins required for virion assembly. In lentiviral systems packaging plasmid(s) provide gag, which encodes the matrix, capsid and nucleocapsid proteins, pol, which encodes reverse transcriptase and integrase, and rev which encodes the protein which binds to the rev response element (RRE) to facilitate nuclear export. Second generation lentiviral systems use a single packaging plasmid encoding gag, pol, rev and also tat, which encodes a transactivator protein for activating transcription from the promoter of the 5′ LTR. In third generation lentiviral systems gag, pol and rev genes are encoded by two separate packaging plasmids. Such systems do not typically employ tat (the 5′ LTR of the transfer plasmid in such systems comprises a promoter which is not tat-dependent).

The envelope plasmid provides nucleic acid encoding the viral envelope glycoprotein.

The viral vector used in the methods of the present invention may be prepared by co-transfection and expression from cells of a plasmid encoding a nucleic acid of interest, a plasmid encoding gag and pol, a plasmid encoding rev, and a plasmid encoding a composite viral envelope glycoprotein as described herein.

In some embodiments the viral vector used in the methods of the present invention may be prepared such that the viral vector comprises a composite viral envelope glycoprotein and a viral envelope glycoprotein.

In some embodiments the viral vector is produced by a method comprising transfecting cells with particular proportions/ratios of nucleic acid encoding the composite viral envelope glycoprotein and viral envelope glycoprotein.

In some embodiments one of greater than 5%, e.g. greater than 10%, 15%, 20%, 25%, 30%, 35%, 40%, 45%, 50%, 55%, 60%, 65%, 70%, 75%, 80%, 85%, 90% or 95% of the nucleic acid encoding a viral envelope glycoprotein used to produce the viral vector used in the methods of the present invention encodes a composite viral envelope glycoprotein.

In some embodiments one of less than 100%, e.g. less than 95%, 90%, 85%, 80%, 75%, 70%, 65%, 60%, 55%, 50%, 45%, 40%, 35%, 30%, 25%, 20%, 15%, 10% or 5% of the nucleic acid encoding a viral envelope glycoprotein used to produce the viral vector used in the methods of the present invention encodes a composite viral envelope glycoprotein.

In some embodiments one of greater than 5% and less than 100%, e.g. greater than 5% and less than 90%, greater than 5% and less than 85%, greater than 10% and less than 80%, greater than 10% and less than 75%, greater than 10% and less than 70%, greater than 15% and less than 65%, greater than 15% and less than 60%, greater than 15% and less than 55%, greater than 20% and less than 50%, greater than 25% and less than 45%, greater than 25% and less than 40% or greater than 30% and less than 35% of the nucleic acid encoding a viral envelope glycoprotein used to produce the viral vector used in the methods of the present invention encodes a composite viral envelope glycoprotein. In some embodiments about 33% of the nucleic acid encoding a viral envelope glycoprotein used to produce the viral vector used in the methods of the present invention encodes a composite viral envelope glycoprotein.

In some embodiments, the ratio of nucleic acid encoding composite viral envelope glycoprotein to nucleic acid viral envelope glycoprotein (e.g. nucleic acid encoding anti-CD3 scFv-VSV-G:nucleic acid encoding VSV-G) used to produce the viral vector used in the methods of the present invention is one of from greater than 1:19 and less than 1:0, e.g. greater than 1:19 and less than 9:1, greater than 1:19 and less than 17:3, greater than 1:9 and less than 4:1, greater than 1:9 and less than 3:1, greater than 1:9 and less than 7:3, greater than 3:17 and less than 13:7, greater than 3:17 and less than 6:4, greater than 3:17 and less than 11:9, greater than 1:4 and less than 1:1, greater than 1:3 and less than 9:11, greater than 1:3 and less than 4:6, or greater than 3:7 and less than 7:13. In some embodiments, the ratio of nucleic acid encoding composite viral envelope glycoprotein to nucleic acid viral envelope glycoprotein (e.g. nucleic acid encoding anti-CD3 scFv-VSV-G:nucleic acid encoding VSV-G) used to produce the viral vector used in the methods of the present invention is about 1:2.

Following transfection, viral vectors and viral particles are produced and released into the culture supernatant of the cells. The viral vectors and viral particles can then be collected and used in subsequent downstream processes, such as the transduction of immune cells.

A representative and simplified viral vector production protocol is as follows:
1. Seed packaging cells at appropriate density in culture vessel with culture media;
2. Incubate cells for approximately 20 hours;
3. Prepare transfection plasmid(s) (e.g. gene of interest plasmid and packaging plasmid(s));
4. Transfect packaging cells with plasmid(s);
5. Incubate cells for growth and expansion;
6. Harvest viruses/viral vectors/particles from the supernatant;
7. Optionally store the viruses/viral vectors/viral particles/viral supernatant (4° C. for several hours but should be frozen in liquid nitrogen and stored at −80° C. as soon as possible).

### Cells for Viral Vector Production

Part of a viral vector production process involves the culture of cells which express a viral vector. These cells are often called packaging cells. Effective packaging cells should be easily transfected and should support high-level expression of viral proteins.

Packaging cells are typically transfected with at least one plasmid. Cells may be transfected with multiple plasmids e.g. with a packaging helper plasmid and a plasmid containing a gene of interest. One or more of these plasmids may contain a viral genome, for example, the gene of interest plasmid may contain the gene of interest and the viral genome.

The skilled person would be aware of many cell types suitable for transfection with viral plasmids (e.g. packaging cells). Mammalian cells and insect cells are excellent hosts for the production of viral vectors and are suitable for transfection with viral plasmids.

Human cells are an example of mammalian cells which are used for the production of viral vectors and are suitable for transfection with viral plasmids. Human cells may be human embryonic kidney cells, HeLa cells, or HT-1080 cells.

Human embryonic kidney (HEK) cells have been widely used in cell biology research for many years, because of their reliable growth and propensity for transfection. Examples of HEK cell lines are HEK293 cells, or cell lines derived from the HEK 293 cell line such as HEK293T, HEK293FT, HEK293S, HEK29FTM, HEK293SG, HEK293SGGD, HEK293H, HEK293E, HEKEBNA1-6E, HEK293MSR and HEK293A cells.

An important variant of the HEK293 cell line is the HEK293T cell line. It contains the SV40 large T-antigen that allows for episomal replication of transfected plasmids containing the SV40 origin of replication. This allows for amplification of transfected plasmids and extended temporal expression of desired gene products. HEK293 and HEK293T cells are commonly used for the production of various viral vectors.

HeLa cells are an immortal cell line used in scientific research. It is the oldest and most commonly used human cell line. HeLa cells can be modified to express viral vectors.

HT-1080 is a fibrosarcoma cell line which has been used extensively in biomedical research. HT-1080 cells can be modified to express viral vectors.

Insect cells can be used for the production of viral vectors. Insect cell lines can be derived from the Orders Lepidoptera (moths and butterflies) and Diptera (flies). Insect cells can be derived from Bombyx mori, Mamestra brassicae, Spodoptera frugiperda, Trichoplusia ni, and Drosophila melanogaster. Examples of insect cell lines are: Sf9, IPLB-Sf21AE (Sf21), Sf900+, Schneider 2 (S2), TN-368, BTI-TN-5B1-4 (High Five), Tni PRO, and Bm-N. Insect cells can be modified to express viral vectors.

### RetroNectin® and Vitronectin

Vitronectin is a glycoprotein found in the extracellular matrix.

The amino acid sequence of Vitronectin from Homo sapiens (including signal peptide) is 478 amino acids long (SEQ ID NO:3). Human vitronectin lacking a signal peptide (P04004) is 459 amino acids in length (SEQ ID NO:2).

The vitronectin sequence is available in Genbank under Accession no. AAH05046.1 GI:13477169.

In this specification “Vitronectin” includes proteins having at least 70%, more preferably one of 75%, 80%, 85%, 90%, 95%, 96%, 97%, 98%, 99% or 100% sequence identity with one or more of the amino acid sequences of vitronectin described above.

The vitronectin protein may be from, or derived from, any animal or human, e.g. non-human animals, e.g. rabbit, guinea pig, rat, mouse or other rodent (including from any animal in the order Rodentia), cat, dog, pig, sheep, goat, cattle (including cows, e.g. dairy cows, or any animal in the order Bos), horse (including any animal in the order Equidae), donkey, and non-human primate or other non-human vertebrate organism; and/or non-human mammalian animal; and/or human.

RetroNectin® is a recombinant human fibronectin (CH-296) that consists of three functional domains: the human fibronectin cell-binding domain (C-domain), heparin-binding domain (H-domain), and CS-1 sequence domain. The amino acid sequence of RetroNectin® is 574 amino acids long (SEQ ID NO:1).

In this specification “RetroNectin®” includes proteins having at least 70%, more preferably one of 75%, 80%, 85%, 90%, 95%, 96%, 97%, 98%, 99% or 100% sequence identity with the amino acid sequence of RetroNectin® described above.

RetroNectin® is a recombinant human fibronectin fragment which contains three functional domains: (1) an N-terminal cell-binding domain, which binds cells via VLA-5 integrin (this domain contains an RGDS motif and comprises 3 fibronectin type III domains 8, 9 and 10), (2) a heparin-binding domain, comprising 3 fibronectin type III domains 12, 13 and 14, and (3) a C-terminal CS-1 sequence, which binds cells via VLA-4 integrin.

Vitronectin is a 54 kDa glycoprotein which contains three main domains; (1) An N-terminal Somatomedin B domain (residues 1-39) which binds to various proteins such as urokinase-type plasminogen activator receptor, (2) A central domain with hemopexin homology (residues 131-342), and (3) A C-terminal domain (residues 347-459) which contains a heparin-binding domain which interacts with heparin and other glycosaminoglycans, and may contribute to the binding of vitronectin to cell surfaces and extracellular matrix components.

Vitronectin also contains an RGD sequence at residues 45-47. This sequence acts as a recognition site for integrin receptors, in particular αvβ3 and αvβ5 integrins on the surface of cells. Binding of vitronectin to these integrins promotes various cellular processes such as cell adhesion, migration and signalling.

A plasminogen binding site is also located within the first 45 amino acids. Plasminogen is the precursor to the proteolytic enzyme plasmin. Binding of plasminogen to vitronectin facilitates the activation of plasminogen to plasmin, which is responsible for degrading fibrin clots and other extracellular matrix protein. Thus, the functions are mainly regulating fibrinolysis and extracellular matrix remodelling.

The N-terminal region of vitronectin comprises a series of repeating alpha-helical segments that coil around each other. This region Is responsible for the dimerization of vitronectin. It forms a rod-like structure and contributes to the stability of the protein.

Polypeptides Derived from RetroNectin® and Vitronectin

In some aspects, the polypeptide of the present disclosure is a fragment of RetroNectin® or vitronectin, a polypeptide that is derived from RetroNectin® or vitronectin, or a polypeptide that is derived from a fragment of RetroNectin® or vitronectin.

It will be appreciated that the polypeptide derived from an amino acid sequence of interest encompasses peptides/polypeptides comprising or consisting of: (i) an amino acid sequence of a protein of interest, or (ii) an amino acid sequence which is similar to the amino acid sequence of the protein of interest (i.e. an amino acid sequence which is similar to the amino acid sequence of (i)).

In some embodiments the first polypeptide comprises the amino acid sequence of interest, and additionally comprises further amino acids. In some embodiments the first peptide/polypeptide comprises the amino acid sequence of interest and one of 1-3, 1-5, 1-10, 1-15, 1-20, 1-25, 1-30, 1-40 or 1-50 additional amino acids, at one or both ends (i.e. the N- or C-terminus) of the amino acid sequence of interest.

In some embodiments the additional amino acids correspond to the amino acids provided at those positions relative to the amino acid sequence of interest, in the context of the protein from which the amino acid sequence of interest is derived. By way of illustration, where an amino acid sequence of interest corresponds to amino acid positions 20 to 30 of the amino acid sequence of a protein of interest, and wherein the first peptide/polypeptide comprises the amino acid sequence of interest and an additional 5 amino acids at the N-terminus of the amino acid sequence of interest, those additional 5 amino acids may correspond to positions 15 to 19 of the amino acid sequence of the protein of interest.

The polypeptide may comprise more than one peptide/polypeptide chain. For example, the first peptide/polypeptide may be a complex of peptides/polypeptides, e.g. comprising 2, 3, 4, 5 or 6 or more peptides/polypeptides. The peptides/polypeptides may be conjugated to one other, or administered as a composition (e.g. mixture) of two or more peptides/polypeptides that are not conjugated.

As used herein, polypeptides derived from a reference polypeptide, or isoforms, fragments, variants or homologues of a given reference polypeptide may be characterised as having at least 70% sequence identity, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to the amino acid sequence of the reference polypeptide.

A polypeptide derived from a reference polypeptide, generally refers to a polypeptide having an amino acid sequence comprising one or more amino acid substitutions, insertions, deletions or other modifications relative to the amino acid sequence of the reference protein, but retaining a considerable degree of sequence identity (e.g. at least 70%) to the amino acid sequence of the reference protein.

A ‘fragment’ generally refers to a fraction of the reference protein. A fragment is derived from the reference protein.

A fragment of vitronectin may comprise at least, i.e. have a minimum length of, 10, 11, 12, 13, 14, 15, 16, 17, 18, 19, 20, 25, 30, 40, 50, 60, 70, 80, 85, 90, 95, 96, 97, 98 or 99% of the corresponding full length sequence. The fragment may have a maximum length, i.e. be no longer than, 10, 11, 12, 13, 14, 15, 16, 17, 18, 19, 20, 25, 30, 40, 50, 60, 70, 80, 85, 90, 95, 96, 97, 98 or 99% of the corresponding full length sequence. The fragment may comprise at least, i.e. have a minimum length of 5 amino acids or one of at least 10, 15, 20, 25, 30, 40, 50, 100, 150, 200, 250, 300, 350, 400, or 450 amino acids. The fragment may have a maximum length of, i.e. be no longer than, 10 amino acids, or one of less than 15, 20, 25, 30, 40, 50, 100, 150, 200, 250, 300, 350, 400, 450 amino acids. The fragment may have a length anywhere between the said minimum and maximum length.

A fragment of RetroNectin® may comprise at least, i.e. have a minimum length of, 10, 11, 12, 13, 14, 15, 16, 17, 18, 19, 20, 25, 30, 40, 50, 60, 70, 80, 85, 90, 95, 96, 97, 98 or 99% of the corresponding full length sequence. The fragment may have a maximum length, i.e. be no longer than, 10, 11, 12, 13, 14, 15, 16, 17, 18, 19, 20, 25, 30, 40, 50, 60, 70, 80, 85, 90, 95, 96, 97, 98 or 99% of the corresponding full length sequence. The fragment may comprise at least, i.e. have a minimum length of 5 amino acids or one of at least 10, 15, 20, 25, 30, 40, 50, 100, 150, 200, 250, 300, 350, 400, 450, 500, or 550 amino acids. The fragment may have a maximum length of, i.e. be no longer than, 10 amino acids, or one of less than 15, 20, 25, 30, 40, 50, 100, 150, 200, 250, 300, 350, 400, 450, 500, or 550 amino acids. The fragment may have a length anywhere between the said minimum and maximum length.

An ‘isoform’ generally refers to a variant of the reference protein expressed by the same species as the species of the reference protein. A ‘homologue’ generally refers to a variant of the reference protein produced by a different species as compared to the species of the reference protein. Homologues include orthologues.

Isoforms, fragments, variants or homologues of a given reference protein may optionally be characterised as having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to the amino acid sequence of an immature or mature (i.e. after processing to remove signal peptide) form of a specified isoform of the relevant polypeptide from a given species, e.g. human.

In some aspects, the polypeptide is derived from RetroNectin® or vitronectin. In some aspects, the polypeptide is a fragment of RetroNectin® or vitronectin. In some aspects, the polypeptide is derived from a fragment of RetroNectin® or vitronectin.

In some aspects, the polypeptide is derived from SEQ ID NO:3, SEQ ID NO:2, and/or SEQ ID NO:1. In some aspects, the polypeptide is a fragment of SEQ ID NO:3, SEQ ID NO:2, and/or SEQ ID NO:1. In some aspects, the polypeptide is derived from a fragment of SEQ ID NO:3, SEQ ID NO:2, and/or SEQ ID NO:1.

By way of example, P1 (SEQ ID NO:6) is a fragment of SEQ ID NO:3, and therefore P1 (SEQ ID NO:6) is derived from SEQ ID NO:3.

By further way of example, P6 (SEQ ID NO:9) is derived from SEQ ID NO:10, and SEQ ID NO:10 is a fragment of SEQ ID NO:3. Therefore, P6 is derived from a fragment of vitronectin.

In some aspects, the polypeptide comprises an amino acid sequence having at least 70% sequence identity to a fragment of SEQ ID NO:3, SEQ ID NO:2, or SEQ ID NO:1.

By way of example, P6 (SEQ ID NO:9) comprises an amino acid sequence having over 70% sequence identity to a fragment of SEQ ID NO:3. It can be seen that SEQ ID NO:10 is a fragment of SEQ ID NO:3, and P6 (SEQ ID NO:9) comprises an amino acid sequence having over 70% sequence identity to SEQ ID NO:10. P6 (SEQ ID NO:9) comprises further amino acids at the N-terminus and C-terminus, and comprises an insertion, relative to SEQ ID NO:10, but P6 (SEQ ID NO:9) comprises an amino acid sequence having over 70% sequence identity to SEQ ID NO:10. By completing a sequence alignment, it can be seen that SEQ ID NO:9 and SEQ ID NO:10 have a percentage Identity of 92.86%.

In some embodiments, the polypeptide derived from SEQ ID NO:1, SEQ ID NO:2, and/or SEQ ID NO:3 is SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, or SEQ ID NO:23.

In some embodiments, the polypeptide derived from SEQ ID NO:1, SEQ ID NO:2, and/or SEQ ID NO:3 is SEQ ID NO:6. In some embodiments, the polypeptide derived from SEQ ID NO:1, SEQ ID NO:2, and/or SEQ ID NO:3 is SEQ ID NO:7. In some embodiments, the polypeptide derived from SEQ ID NO:1, SEQ ID NO:2, and/or SEQ ID NO:3 is SEQ ID NO:8. In some embodiments, the polypeptide derived from SEQ ID NO:1, SEQ ID NO:2, and/or SEQ ID NO:3 is SEQ ID NO:9. In some embodiments, the polypeptide derived from SEQ ID NO:1, SEQ ID NO:2, and/or SEQ ID NO:3 is SEQ ID NO:10. In some embodiments, the polypeptide derived from SEQ ID NO:1, SEQ ID NO:2, and/or SEQ ID NO:3 is SEQ ID NO:11. In some embodiments, the polypeptide derived from SEQ ID NO:1, SEQ ID NO:2, and/or SEQ ID NO:3 is SEQ ID NO:12. In some embodiments, the polypeptide derived from SEQ ID NO:1, SEQ ID NO:2, and/or SEQ ID NO:3 is SEQ ID NO:13. In some embodiments, the polypeptide derived from SEQ ID NO:1, SEQ ID NO:2, and/or SEQ ID NO:3 is SEQ ID NO:14. In some embodiments, the polypeptide derived from SEQ ID NO:1, SEQ ID NO:2, and/or SEQ ID NO:3 is SEQ ID NO:15. In some embodiments, the polypeptide derived from SEQ ID NO:1, SEQ ID NO:2, and/or SEQ ID NO:3 is SEQ ID NO:16. In some embodiments, the polypeptide derived from SEQ ID NO:1, SEQ ID NO:2, and/or SEQ ID NO:3 is SEQ ID NO:17. In some embodiments, the polypeptide derived from SEQ ID NO:1, SEQ ID NO:2, and/or SEQ ID NO:3 is SEQ ID NO:18. In some embodiments, the polypeptide derived from SEQ ID NO:1, SEQ ID NO:2, and/or SEQ ID NO:3 is SEQ ID NO:19. In some embodiments, the polypeptide derived from SEQ ID NO:1, SEQ ID NO:2, and/or SEQ ID NO:3 is SEQ ID NO:20. In some embodiments, the polypeptide derived from SEQ ID NO:1, SEQ ID NO:2, and/or SEQ ID NO:3 is SEQ ID NO:21. In some embodiments, the polypeptide derived from SEQ ID NO:1, SEQ ID NO:2, and/or SEQ ID NO:3 is SEQ ID NO:22. In some embodiments, the polypeptide derived from SEQ ID NO:1, SEQ ID NO:2, and/or SEQ ID NO:3 is SEQ ID NO:23.

In some embodiments, the polypeptide derived from SEQ ID NO:1, SEQ ID NO:2, and/or SEQ ID NO:3 is SEQ ID NO:6, SEQ ID NO:7, SEQ ID NO:9, SEQ ID NO:12, SEQ ID NO:13, SEQ ID NO:14, SEQ ID NO:15, SEQ ID NO:16, or SEQ ID NO:17.

In some embodiments, the polypeptide derived from SEQ ID NO:1, SEQ ID NO:2, and/or SEQ ID NO:3 is SEQ ID NO:6, SEQ ID NO:7, SEQ ID NO:9, SEQ ID NO:12, SEQ ID NO:16, or SEQ ID NO:17.

In some embodiments, the polypeptide derived from a fragment of SEQ ID NO:1, SEQ ID NO:2, and/or SEQ ID NO:3 is SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, or SEQ ID NO:23.

In some embodiments, the polypeptide derived from a fragment of SEQ ID NO:1, SEQ ID NO:2, and/or SEQ ID NO:3 is SEQ ID NO:6. In some embodiments, the polypeptide derived from a fragment of SEQ ID NO:1, SEQ ID NO:2, and/or SEQ ID NO:3 is SEQ ID NO:7. In some embodiments, the polypeptide derived from a fragment of SEQ ID NO:1, SEQ ID NO:2, and/or SEQ ID NO:3 is SEQ ID NO:8. In some embodiments, the polypeptide derived from a fragment of SEQ ID NO:1, SEQ ID NO:2, and/or SEQ ID NO:3 is SEQ ID NO:9. In some embodiments, the polypeptide derived from a fragment of SEQ ID NO:1, SEQ ID NO:2, and/or SEQ ID NO:3 is SEQ ID NO:10. In some embodiments, the polypeptide derived from a fragment of SEQ ID NO:1, SEQ ID NO:2, and/or SEQ ID NO:3 is SEQ ID NO:11. In some embodiments, the polypeptide derived from a fragment of SEQ ID NO:1, SEQ ID NO:2, and/or SEQ ID NO:3 is SEQ ID NO:12. In some embodiments, the polypeptide derived from a fragment of SEQ ID NO:1, SEQ ID NO:2, and/or SEQ ID NO:3 is SEQ ID NO:13. In some embodiments, the polypeptide derived from a fragment of SEQ ID NO:1, SEQ ID NO:2, and/or SEQ ID NO:3 is SEQ ID NO:14. In some embodiments, the polypeptide derived from a fragment of SEQ ID NO:1, SEQ ID NO:2, and/or SEQ ID NO:3 is SEQ ID NO:15. In some embodiments, the polypeptide derived from a fragment of SEQ ID NO:1, SEQ ID NO:2, and/or SEQ ID NO:3 is SEQ ID NO:16. In some embodiments, the polypeptide derived from a fragment of SEQ ID NO:1, SEQ ID NO:2, and/or SEQ ID NO:3 is SEQ ID NO:17. In some embodiments, the polypeptide derived from a fragment of SEQ ID NO:1, SEQ ID NO:2, and/or SEQ ID NO:3 is SEQ ID NO:18. In some embodiments, the polypeptide derived from a fragment of SEQ ID NO:1, SEQ ID NO:2, and/or SEQ ID NO:3 is SEQ ID NO:19. In some embodiments, the polypeptide derived from a fragment of SEQ ID NO:1, SEQ ID NO:2, and/or SEQ ID NO:3 is SEQ ID NO:20. In some embodiments, the polypeptide derived from a fragment of SEQ ID NO:1, SEQ ID NO:2, and/or SEQ ID NO:3 is SEQ ID NO:21. In some embodiments, the polypeptide derived from a fragment of SEQ ID NO:1, SEQ ID NO:2, and/or SEQ ID NO:3 is SEQ ID NO:22. In some embodiments, the polypeptide derived from a fragment of SEQ ID NO:1, SEQ ID NO:2, and/or SEQ ID NO:3 is SEQ ID NO:23.

In some embodiments, the polypeptide derived from a fragment of SEQ ID NO:1, SEQ ID NO:2, and/or SEQ ID NO:3 is SEQ ID NO:6, SEQ ID NO:7, SEQ ID NO:9, SEQ ID NO:12, SEQ ID NO:13, SEQ ID NO:14, SEQ ID NO:15, SEQ ID NO:16, or SEQ ID NO:17.

In some embodiments, the polypeptide derived from a fragment of SEQ ID NO:1, SEQ ID NO:2, and/or SEQ ID NO:3 is SEQ ID NO:6, SEQ ID NO:7, SEQ ID NO:9, SEQ ID NO:12, SEQ ID NO:16, or SEQ ID NO:17.

In some embodiments, the fragment of vitronectin comprises amino acids derived from the N-terminal Somatomedin B domain of vitronectin. In some embodiments, the fragment of vitronectin comprises amino acids derived from the central domain of vitronectin. In some embodiments, the fragment of vitronectin comprises amino acids derived from the C-terminal domain of vitronectin.

In some embodiments, the fragment of vitronectin comprises an amino acid sequence having over 70% sequence identity to an amino acid sequence taken from the N-terminal Somatomedin B domain of vitronectin. In some embodiments, the fragment of vitronectin comprises an amino acid sequence having over 70% sequence identity to an amino acid sequence taken from the central domain of vitronectin. In some embodiments, the fragment of vitronectin comprises an amino acid sequence having over 70% sequence identity to an amino acid sequence taken from the C-terminal domain of vitronectin.

In some embodiments, the fragment of vitronectin comprises amino acids from the N-terminal Somatomedin B domain of vitronectin. In some embodiments, the fragment of vitronectin comprises amino acids from the central domain of vitronectin. In some embodiments, the fragment of vitronectin comprises amino acids from the C-terminal domain of vitronectin.

In some embodiments, the fragment of vitronectin comprises the N-terminal Somatomedin B domain of vitronectin. In some embodiments, the fragment of vitronectin comprises the central domain of vitronectin. In some embodiments, the fragment of vitronectin comprises the C-terminal domain of vitronectin.

In some embodiments, the fragment of SEQ ID NO:3 and/or SEQ ID NO:2 comprises amino acids derived from the N-terminal Somatomedin B domain of vitronectin. In some embodiments, the fragment of SEQ ID NO:3 and/or SEQ ID NO:2 comprises amino acids derived from the central domain of vitronectin. In some embodiments, the SEQ ID NO:3 and/or SEQ ID NO:2 of vitronectin comprises amino acids derived from the C-terminal domain of vitronectin.

In some embodiments, the fragment of SEQ ID NO:3 and/or SEQ ID NO:2 comprises an amino acid sequence having over 70% sequence identity to an amino acid sequence taken from the N-terminal Somatomedin B domain of vitronectin. In some embodiments, the fragment of SEQ ID NO:3 and/or SEQ ID NO:2 comprises an amino acid sequence having over 70% sequence identity to an amino acid sequence taken from the central domain of vitronectin. In some embodiments, the fragment of SEQ ID NO:3 and/or SEQ ID NO:2 comprises an amino acid sequence having over 70% sequence identity to an amino acid sequence taken from the C-terminal domain of vitronectin.

In some embodiments, the fragment of SEQ ID NO:3 and/or SEQ ID NO:2 comprises amino acids from the N-terminal Somatomedin B domain of vitronectin. In some embodiments, the fragment of SEQ ID NO:3 and/or SEQ ID NO:2 comprises amino acids from the central domain of vitronectin. In some embodiments, the fragment of SEQ ID NO:3 and/or SEQ ID NO:2 comprises amino acids from the C-terminal domain of vitronectin.

In some embodiments, the fragment of SEQ ID NO:3 and/or SEQ ID NO:2 comprises the N-terminal Somatomedin B domain of vitronectin. In some embodiments, the fragment of SEQ ID NO:3 and/or SEQ ID NO:2 comprises the central domain of vitronectin. In some embodiments, the fragment of SEQ ID NO:3 and/or SEQ ID NO:2 comprises the C-terminal domain of vitronectin.

In some embodiments, the fragment of RetroNectin® comprises amino acids derived from the N-terminal cell-binding domain of RetroNectin®. In some embodiments, the fragment of RetroNectin® comprises amino acids derived from the heparin-binding domain of RetroNectin®. In some embodiments, the fragment of RetroNectin® comprises amino acids derived from the C-terminal CS-1 sequence of RetroNectin®.

In some embodiments, the fragment of RetroNectin® comprises an amino acid sequence having over 70% sequence identity to an amino acid sequence taken from the N-terminal cell-binding domain of RetroNectin®. In some embodiments, the fragment of RetroNectin® comprises an amino acid sequence having over 70% sequence identity to an amino acid sequence taken from the heparin-binding domain RetroNectin®. In some embodiments, the fragment of RetroNectin® comprises an amino acid sequence having over 70% sequence identity to an amino acid sequence taken from the C-terminal CS-1 sequence of RetroNectin®.

In some embodiments, the fragment of RetroNectin® comprises an amino acid sequence taken from the N-terminal cell-binding domain of RetroNectin®. In some embodiments, the fragment of RetroNectin® comprises an amino acid sequence taken from the heparin-binding domain of RetroNectin®. In some embodiments, the fragment of RetroNectin® comprises an amino acid sequence taken from the C-terminal CS-1 sequence of RetroNectin®.

In some embodiments, the fragment of RetroNectin® comprises the N-terminal cell-binding domain of RetroNectin®. In some embodiments, the fragment of RetroNectin® comprises the heparin-binding domain of RetroNectin®. In some embodiments, the fragment of RetroNectin® comprises the C-terminal CS-1 sequence of RetroNectin®.

In some embodiments, the fragment of SEQ ID NO:1 comprises amino acids derived from the N-terminal cell-binding domain of RetroNectin®. In some embodiments, the fragment of SEQ ID NO:1 comprises amino acids derived from the heparin-binding domain of RetroNectin®. In some embodiments, the fragment of SEQ ID NO:1 comprises amino acids derived from the C-terminal CS-1 sequence of RetroNectin®.

In some embodiments, the fragment of SEQ ID NO:1 comprises an amino acid sequence having over 70% sequence identity to an amino acid sequence taken from the N-terminal cell-binding domain of RetroNectin®. In some embodiments, the fragment of SEQ ID NO:1 comprises an amino acid sequence having over 70% sequence identity to an amino acid sequence taken from the heparin-binding domain RetroNectin®. In some embodiments, the fragment of SEQ ID NO:1 comprises an amino acid sequence having over 70% sequence identity to an amino acid sequence taken from the C-terminal CS-1 sequence of RetroNectin®.

In some embodiments, the fragment of SEQ ID NO:1 comprises an amino acid sequence taken from the N-terminal cell-binding domain of RetroNectin®. In some embodiments, the fragment of SEQ ID NO:1 comprises an amino acid sequence taken from the heparin-binding domain of RetroNectin®. In some embodiments, the fragment of SEQ ID NO:1 comprises an amino acid sequence taken from the C-terminal CS-1 sequence of RetroNectin®.

In some embodiments, the fragment of SEQ ID NO:1 comprises the N-terminal cell-binding domain of RetroNectin®. In some embodiments, the fragment of SEQ ID NO:1 comprises the heparin-binding domain of RetroNectin®. In some embodiments, the fragment of SEQ ID NO:1 comprises the C-terminal CS-1 sequence of RetroNectin®.

In some embodiments, the fragment of SEQ ID NO:1, SEQ ID NO:2, and/or SEQ ID NO:3 is SEQ ID NO:6, SEQ ID NO:8, SEQ ID NO:10, SEQ ID NO:12, SEQ ID NO:13, SEQ ID NO:14, SEQ ID NO:15, SEQ ID NO:19, SEQ ID NO:20, SEQ ID NO:21, SEQ ID NO:22, or SEQ ID NO:23.

In some embodiments, the fragment of SEQ ID NO:1, SEQ ID NO:2, and/or SEQ ID NO:3 is SEQ ID NO:6. In some embodiments, the fragment of SEQ ID NO:1, SEQ ID NO:2, and/or SEQ ID NO:3 is SEQ ID NO:8. In some embodiments, the fragment of SEQ ID NO:1, SEQ ID NO:2, and/or SEQ ID NO:3 is SEQ ID NO:10. In some embodiments, the fragment of SEQ ID NO:1, SEQ ID NO:2, and/or SEQ ID NO:3 is SEQ ID NO:12. In some embodiments, the fragment of SEQ ID NO:1, SEQ ID NO:2, and/or SEQ ID NO:3 is SEQ ID NO:13. In some embodiments, the fragment of SEQ ID NO:1, SEQ ID NO:2, and/or SEQ ID NO:3 is SEQ ID NO:14. In some embodiments, the fragment of SEQ ID NO:1, SEQ ID NO:2, and/or SEQ ID NO:3 is SEQ ID NO:15. In some embodiments, the fragment of SEQ ID NO:1, SEQ ID NO:2, and/or SEQ ID NO:3 is SEQ ID NO:19. In some embodiments, the fragment of SEQ ID NO:1, SEQ ID NO:2, and/or SEQ ID NO:3 is SEQ ID NO:20. In some embodiments, the fragment of SEQ ID NO:1, SEQ ID NO:2, and/or SEQ ID NO:3 is SEQ ID NO:21. In some embodiments, the fragment of SEQ ID NO:1, SEQ ID NO:2, and/or SEQ ID NO:3 is SEQ ID NO:22. In some embodiments, the fragment of SEQ ID NO:1, SEQ ID NO:2, and/or SEQ ID NO:3 is SEQ ID NO:23.

In some embodiments, the polypeptide has at least 70% sequence identity to one of SEQ ID NO:3, SEQ ID NO:2, SEQ ID NO:1, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, and/or SEQ ID NO:23, preferably wherein the polypeptide has one of at least 75%, 80%, 85%, 90%, 95%, or 99% sequence identity to one of SEQ ID NO:3, SEQ ID NO:2, SEQ ID NO:1, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, and/or SEQ ID NO:23.

In some embodiments, the polypeptide has at least 70% sequence identity to one of SEQ ID NO:3, SEQ ID NO:2, SEQ ID NO:1, SEQ ID NO:6, SEQ ID NO:7, SEQ ID NO:9, SEQ ID NO:12, SEQ ID NO:13, SEQ ID NO:14, SEQ ID NO:15, SEQ ID NO:16, and/or SEQ ID NO:17, preferably wherein the polypeptide has one of at least 75%, 80%, 85%, 90%, 95%, or 99% sequence identity to one of SEQ ID NO:3, SEQ ID NO:2, SEQ ID NO:1, SEQ ID NO:6, SEQ ID NO:7, SEQ ID NO:9, SEQ ID NO:12, SEQ ID NO:13, SEQ ID NO:14, SEQ ID NO:15, SEQ ID NO:16, and/or SEQ ID NO:17.

In some embodiments, the polypeptide has at least 70% sequence identity to one of SEQ ID NO:3, SEQ ID NO:2, SEQ ID NO:1, SEQ ID NO:6, SEQ ID NO:7, SEQ ID NO:9, SEQ ID NO:12, SEQ ID NO:16, and/or SEQ ID NO:17, preferably wherein the polypeptide has one of at least 75%, 80%, 85%, 90%, 95%, or 99% sequence identity to one of SEQ ID NO:3, SEQ ID NO:2, SEQ ID NO:1, SEQ ID NO:6, SEQ ID NO:7, SEQ ID NO:9, SEQ ID NO:12, SEQ ID NO:16, and/or SEQ ID NO:17.

In some embodiments, the polypeptide has at least 70% sequence identity to one of SEQ ID NO:3, SEQ ID NO:2, SEQ ID NO:1, SEQ ID NO:6, SEQ ID NO:7, SEQ ID NO:9, and/or SEQ ID NO:12, preferably wherein the polypeptide has one of at least 75%, 80%, 85%, 90%, 95%, or 99% sequence identity to one of SEQ ID NO:3, SEQ ID NO:2, SEQ ID NO:1, SEQ ID NO:6, SEQ ID NO:7, SEQ ID NO:9, and/or SEQ ID NO:12.

In some embodiments, the polypeptide has at least 70% sequence identity to one of SEQ ID NO:6, SEQ ID NO:7, SEQ ID NO:9, and/or SEQ ID NO:12, preferably wherein the polypeptide has one of at least 75%, 80%, 85%, 90%, 95%, or 99% sequence identity to one of SEQ ID NO:6, SEQ ID NO:7, SEQ ID NO:9, and/or SEQ ID NO:12.

In some embodiments, the polypeptide has at least 70% sequence identity to SEQ ID NO:3. In some embodiments, the polypeptide has at least 70% sequence identity to SEQ ID NO:2. In some embodiments, the polypeptide has at least 70% sequence identity to SEQ ID NO:1. In some embodiments, the polypeptide has at least 70% sequence identity to SEQ ID NO:4. In some embodiments, the polypeptide has at least 70% sequence identity to SEQ ID NO:5. In some embodiments, the polypeptide has at least 70% sequence identity to SEQ ID NO:6. In some embodiments, the polypeptide has at least 70% sequence identity to SEQ ID NO:7. In some embodiments, the polypeptide has at least 70% sequence identity to SEQ ID NO:8. In some embodiments, the polypeptide has at least 70% sequence identity to SEQ ID NO:9. In some embodiments, the polypeptide has at least 70% sequence identity to SEQ ID NO:10. In some embodiments, the polypeptide has at least 70% sequence identity to SEQ ID NO:11. In some embodiments, the polypeptide has at least 70% sequence identity to SEQ ID NO:12. In some embodiments, the polypeptide has at least 70% sequence identity to SEQ ID NO:13. In some embodiments, the polypeptide has at least 70% sequence identity to SEQ ID NO:14. In some embodiments, the polypeptide has at least 70% sequence identity to SEQ ID NO:15. In some embodiments, the polypeptide has at least 70% sequence identity to SEQ ID NO:16. In some embodiments, the polypeptide has at least 70% sequence identity to SEQ ID NO:17. In some embodiments, the polypeptide has at least 70% sequence identity to SEQ ID NO:18. In some embodiments, the polypeptide has at least 70% sequence identity to SEQ ID NO:19. In some embodiments, the polypeptide has at least 70% sequence identity to SEQ ID NO:20. In some embodiments, the polypeptide has at least 70% sequence identity to SEQ ID NO:21. In some embodiments, the polypeptide has at least 70% sequence identity to SEQ ID NO:22. In some embodiments, the polypeptide has at least 70% sequence identity to SEQ ID NO:23.

In some embodiments, the polypeptide has at least 90% sequence identity to SEQ ID NO:3. In some embodiments, the polypeptide has at least 90% sequence identity to SEQ ID NO:2. In some embodiments, the polypeptide has at least 90% sequence identity to SEQ ID NO:1. In some embodiments, the polypeptide has at least 90% sequence identity to SEQ ID NO:4. In some embodiments, the polypeptide has at least 90% sequence identity to SEQ ID NO:5. In some embodiments, the polypeptide has at least 90% sequence identity to SEQ ID NO:6. In some embodiments, the polypeptide has at least 90% sequence identity to SEQ ID NO:7. In some embodiments, the polypeptide has at least 90% sequence identity to SEQ ID NO:8. In some embodiments, the polypeptide has at least 90% sequence identity to SEQ ID NO:9. In some embodiments, the polypeptide has at least 90% sequence identity to SEQ ID NO:10. In some embodiments, the polypeptide has at least 90% sequence identity to SEQ ID NO:11. In some embodiments, the polypeptide has at least 90% sequence identity to SEQ ID NO:12. In some embodiments, the polypeptide has at least 90% sequence identity to SEQ ID NO:13. In some embodiments, the polypeptide has at least 90% sequence identity to SEQ ID NO:14. In some embodiments, the polypeptide has at least 90% sequence identity to SEQ ID NO:15. In some embodiments, the polypeptide has at least 90% sequence identity to SEQ ID NO:16. In some embodiments, the polypeptide has at least 90% sequence identity to SEQ ID NO:17. In some embodiments, the polypeptide has at least 90% sequence identity to SEQ ID NO:18. In some embodiments, the polypeptide has at least 90% sequence identity to SEQ ID NO:19. In some embodiments, the polypeptide has at least 90% sequence identity to SEQ ID NO:20. In some embodiments, the polypeptide has at least 90% sequence identity to SEQ ID NO:21. In some embodiments, the polypeptide has at least 90% sequence identity to SEQ ID NO:22. In some embodiments, the polypeptide has at least 90% sequence identity to SEQ ID NO:23.

In some embodiments, the polypeptide has at least 75%, 80%, 85%, 90%, 95%, or 99% sequence identity to SEQ ID NO:3. In some embodiments, the polypeptide has at least 75%, 80%, 85%, 90%, 95%, or 99% sequence identity to SEQ ID NO:2. In some embodiments, the polypeptide has at least 75%, 80%, 85%, 90%, 95%, or 99% sequence identity to SEQ ID NO:1. In some embodiments, the polypeptide has at least 75%, 80%, 85%, 90%, 95%, or 99% sequence identity to SEQ ID NO:4. In some embodiments, the polypeptide has at least 75%, 80%, 85%, 90%, 95%, or 99% sequence identity to SEQ ID NO:5. In some embodiments, the polypeptide has at least 75%, 80%, 85%, 90%, 95%, or 99% sequence identity to SEQ ID NO:6. In some embodiments, the polypeptide has at least 75%, 80%, 85%, 90%, 95%, or 99% sequence identity to SEQ ID NO:7. In some embodiments, the polypeptide has at least 75%, 80%, 85%, 90%, 95%, or 99% sequence identity to SEQ ID NO:8. In some embodiments, the polypeptide has at least 75%, 80%, 85%, 90%, 95%, or 99% sequence identity to SEQ ID NO:9. In some embodiments, the polypeptide has at least 75%, 80%, 85%, 90%, 95%, or 99% sequence identity to SEQ ID NO:10. In some embodiments, the polypeptide has at least 75%, 80%, 85%, 90%, 95%, or 99% sequence identity to SEQ ID NO:11. In some embodiments, the polypeptide has at least 75%, 80%, 85%, 90%, 95%, or 99% sequence identity to SEQ ID NO:12. In some embodiments, the polypeptide has at least 75%, 80%, 85%, 90%, 95%, or 99% sequence identity to SEQ ID NO:13. In some embodiments, the polypeptide has at least 75%, 80%, 85%, 90%, 95%, or 99% sequence identity to SEQ ID NO:14. In some embodiments, the polypeptide has at least 75%, 80%, 85%, 90%, 95%, or 99% sequence identity to SEQ ID NO:15. In some embodiments, the polypeptide has at least 75%, 80%, 85%, 90%, 95%, or 99% sequence identity to SEQ ID NO:16. In some embodiments, the polypeptide has at least 75%, 80%, 85%, 90%, 95%, or 99% sequence identity to SEQ ID NO:17. In some embodiments, the polypeptide has at least 75%, 80%, 85%, 90%, 95%, or 99% sequence identity to SEQ ID NO:18. In some embodiments, the polypeptide has at least 75%, 80%, 85%, 90%, 95%, or 99% sequence identity to SEQ ID NO:19. In some embodiments, the polypeptide has at least 75%, 80%, 85%, 90%, 95%, or 99% sequence identity to SEQ ID NO:20. In some embodiments, the polypeptide has at least 75%, 80%, 85%, 90%, 95%, or 99% sequence identity to SEQ ID NO:21. In some embodiments, the polypeptide has at least 75%, 80%, 85%, 90%, 95%, or 99% sequence identity to SEQ ID NO:22. In some embodiments, the polypeptide has at least 75%, 80%, 85%, 90%, 95%, or 99% sequence identity to SEQ ID NO:23.

In some embodiments, the polypeptide comprises an amino acid sequence of SEQ ID NO:3, SEQ ID NO:2, or SEQ ID NO:1, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, and/or SEQ ID NO:23, or an amino acid sequence comprising one, two, three, four, five, six, seven, eight, nine, or ten modifications (e.g. substitutions, insertions, or deletions) relative to the amino acid sequence of SEQ ID NO:3, SEQ ID NO:2, or SEQ ID NO:1, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, and/or SEQ ID NO:23.

In some embodiments, the polypeptide comprises an amino acid sequence of SEQ ID NO:3, SEQ ID NO:2, SEQ ID NO:1, SEQ ID NO:6, SEQ ID NO:7, SEQ ID NO:9, SEQ ID NO:12, SEQ ID NO:13, SEQ ID NO:14, SEQ ID NO:15, SEQ ID NO:16, and/or SEQ ID NO:17, or an amino acid sequence comprising one, two, three, four, five, six, seven, eight, nine, or ten modifications (e.g. substitutions, insertions, or deletions) relative to the amino acid sequence of SEQ ID NO:1, SEQ ID NO:2, SEQ ID NO:3, SEQ ID NO:6, SEQ ID NO:7, SEQ ID NO:9, SEQ ID NO:12, SEQ ID NO:13, SEQ ID NO:14, SEQ ID NO:15, SEQ ID NO:16, and/or SEQ ID NO:17.

In some embodiments, the polypeptide comprises an amino acid sequence of SEQ ID NO:1, SEQ ID NO:2, SEQ ID NO:3, SEQ ID NO:6, SEQ ID NO:7, SEQ ID NO:9, SEQ ID NO:12, SEQ ID NO:16, and/or SEQ ID NO:17, or an amino acid sequence comprising one, two, three, four, five, six, seven, eight, nine, or ten modifications (e.g. substitutions, insertions, or deletions) relative to the amino acid sequence of SEQ ID NO:1, SEQ ID NO:2, SEQ ID NO:3, SEQ ID NO:6, SEQ ID NO:7, SEQ ID NO:9, SEQ ID NO:12, SEQ ID NO:16, and/or SEQ ID NO:17.

In some embodiments, the polypeptide comprises an amino acid sequence of SEQ ID NO:1, SEQ ID NO:2, SEQ ID NO:3, SEQ ID NO:6, SEQ ID NO:7, SEQ ID NO:9, SEQ ID NO:12, SEQ ID NO:16, and/or SEQ ID NO:17, or an amino acid sequence comprising up to 10 modifications (e.g. substitutions, insertions, or deletions) relative to the amino acid sequence of SEQ ID NO:1, SEQ ID NO:2, SEQ ID NO:3, SEQ ID NO:6, SEQ ID NO:7, SEQ ID NO:9, SEQ ID NO:12, SEQ ID NO:16, and/or SEQ ID NO:17.

In some embodiments, the polypeptide comprises an amino acid sequence of SEQ ID NO:1, SEQ ID NO:2, SEQ ID NO:3, SEQ ID NO:6, SEQ ID NO:7, SEQ ID NO:9, SEQ ID NO:12, SEQ ID NO:13, SEQ ID NO:14, SEQ ID NO:15, SEQ ID NO:16, and/or SEQ ID NO:17, or an amino acid sequence comprising up to ten modifications (e.g. substitutions, insertions, or deletions) relative to the amino acid sequence of SEQ ID NO:1, SEQ ID NO:2, SEQ ID NO:3, SEQ ID NO:6, SEQ ID NO:7, SEQ ID NO:9, SEQ ID NO:12, SEQ ID NO:13, SEQ ID NO:14, SEQ ID NO:15, SEQ ID NO:16, and/or SEQ ID NO:17.

In some embodiments, the polypeptide comprises an amino acid sequence of SEQ ID NO:1, SEQ ID NO:2, SEQ ID NO:3, SEQ ID NO:6, SEQ ID NO:7, SEQ ID NO:9, SEQ ID NO:12, SEQ ID NO:16, and/or SEQ ID NO:17, or an amino acid sequence comprising up to ten modifications (e.g. substitutions, insertions, or deletions) relative to the amino acid sequence of SEQ ID NO:1, SEQ ID NO:2, SEQ ID NO:3, SEQ ID NO:6, SEQ ID NO:7, SEQ ID NO:9, SEQ ID NO:12, SEQ ID NO:16, and/or SEQ ID NO:17.

In some embodiments, the polypeptide comprises an amino acid sequence which comprises up to ten modifications (e.g. substitutions, insertions, or deletions) relative to the amino acid sequence of SEQ ID NO:1. In some embodiments, the polypeptide comprises an amino acid sequence which comprises up to ten modifications (e.g. substitutions, insertions, or deletions) relative to the amino acid sequence of SEQ ID NO:2. In some embodiments, the polypeptide comprises an amino acid sequence which comprises up to ten modifications (e.g. substitutions, insertions, or deletions) relative to the amino acid sequence of SEQ ID NO:3. In some embodiments, the polypeptide comprises an amino acid sequence which comprises up to ten modifications (e.g. substitutions, insertions, or deletions) relative to the amino acid sequence of SEQ ID NO:4. In some embodiments, the polypeptide comprises an amino acid sequence which comprises up to ten modifications (e.g. substitutions, insertions, or deletions) relative to the amino acid sequence of SEQ ID NO:5. In some embodiments, the polypeptide comprises an amino acid sequence which comprises up to ten modifications (e.g. substitutions, insertions, or deletions) relative to the amino acid sequence of SEQ ID NO:6. In some embodiments, the polypeptide comprises an amino acid sequence which comprises up to ten modifications (e.g. substitutions, insertions, or deletions) relative to the amino acid sequence of SEQ ID NO:7. In some embodiments, the polypeptide comprises an amino acid sequence which comprises up to ten modifications (e.g. substitutions, insertions, or deletions) relative to the amino acid sequence of SEQ ID NO:8. In some embodiments, the polypeptide comprises an amino acid sequence which comprises up to ten modifications (e.g. substitutions, insertions, or deletions) relative to the amino acid sequence of SEQ ID NO:9. In some embodiments, the polypeptide comprises an amino acid sequence which comprises up to ten modifications (e.g. substitutions, insertions, or deletions) relative to the amino acid sequence of SEQ ID NO:10. In some embodiments, the polypeptide comprises an amino acid sequence which comprises up to ten modifications (e.g. substitutions, insertions, or deletions) relative to the amino acid sequence of SEQ ID NO:11. In some embodiments, the polypeptide comprises an amino acid sequence which comprises up to ten modifications (e.g. substitutions, insertions, or deletions) relative to the amino acid sequence of SEQ ID NO:12. In some embodiments, the polypeptide comprises an amino acid sequence which comprises up to ten modifications (e.g. substitutions, insertions, or deletions) relative to the amino acid sequence of SEQ ID NO:13. In some embodiments, the polypeptide comprises an amino acid sequence which comprises up to ten modifications (e.g. substitutions, insertions, or deletions) relative to the amino acid sequence of SEQ ID NO:14. In some embodiments, the polypeptide comprises an amino acid sequence which comprises up to ten modifications (e.g. substitutions, insertions, or deletions) relative to the amino acid sequence of SEQ ID NO:15. In some embodiments, the polypeptide comprises an amino acid sequence which comprises up to ten modifications (e.g. substitutions, insertions, or deletions) relative to the amino acid sequence of SEQ ID NO:16. In some embodiments, the polypeptide comprises an amino acid sequence which comprises up to ten modifications (e.g. substitutions, insertions, or deletions) relative to the amino acid sequence of SEQ ID NO:17. In some embodiments, the polypeptide comprises an amino acid sequence which comprises up to ten modifications (e.g. substitutions, insertions, or deletions) relative to the amino acid sequence of SEQ ID NO:18. In some embodiments, the polypeptide comprises an amino acid sequence which comprises up to ten modifications (e.g. substitutions, insertions, or deletions) relative to the amino acid sequence of SEQ ID NO:19. In some embodiments, the polypeptide comprises an amino acid sequence which comprises up to ten modifications (e.g. substitutions, insertions, or deletions) relative to the amino acid sequence of SEQ ID NO:20. In some embodiments, the polypeptide comprises an amino acid sequence which comprises up to ten modifications (e.g. substitutions, insertions, or deletions) relative to the amino acid sequence of SEQ ID NO:21. In some embodiments, the polypeptide comprises an amino acid sequence which comprises up to ten modifications (e.g. substitutions, insertions, or deletions) relative to the amino acid sequence of SEQ ID NO:22. In some embodiments, the polypeptide comprises an amino acid sequence which comprises up to ten modifications (e.g. substitutions, insertions, or deletions) relative to the amino acid sequence of SEQ ID NO:23.

In some embodiments, the polypeptide comprises an amino acid sequence which comprises up to 1 modification (e.g. substitutions, insertions, or deletions) relative to the reference polypeptide.

In some embodiments, the polypeptide comprises an amino acid sequence which comprises up to 2 modifications (e.g. substitutions, insertions, or deletions) relative to the reference polypeptide.

In some embodiments, the polypeptide comprises an amino acid sequence which comprises up to 3 modifications (e.g. substitutions, insertions, or deletions) relative to the reference polypeptide.

In some embodiments, the polypeptide comprises an amino acid sequence which comprises up to 4 modifications (e.g. substitutions, insertions, or deletions) relative to the reference polypeptide.

In some embodiments, the polypeptide comprises an amino acid sequence which comprises up to 5 modifications (e.g. substitutions, insertions, or deletions) relative to the reference polypeptide.

In some embodiments, the polypeptide comprises an amino acid sequence which comprises up to 6 modifications (e.g. substitutions, insertions, or deletions) relative to the reference polypeptide.

In some embodiments, the polypeptide comprises an amino acid sequence which comprises up to 7 modifications (e.g. substitutions, insertions, or deletions) relative to the reference polypeptide.

In some embodiments, the polypeptide comprises an amino acid sequence which comprises up to 8 modifications (e.g. substitutions, insertions, or deletions) relative to the reference polypeptide.

In some embodiments, the polypeptide comprises an amino acid sequence which comprises up to 9 modifications (e.g. substitutions, insertions, or deletions) relative to the reference polypeptide.

In some embodiments, the polypeptide comprises an amino acid sequence which comprises up to 10 modifications (e.g. substitutions, insertions, or deletions) relative to the reference polypeptide.

In some embodiments the polypeptide is provided as a conjugate with another polypeptide/protein (e.g. an immunoglobulin or a carrier protein).

Polypeptides may be conjugated, joined, or fused to another component. The additional component may be provided to improve a functional property of the polypeptide. For example, the polypeptide may be joined to another component to increase stability, reduce immunogenicity, and/or to increase half-life. In some aspects, polypeptides may be conjugated, joined, or fused to another polypeptide. The polypeptide may be conjugated, joined, or fused to an immunoglobulin.

Immunoglobulins and their structures are described e.g. in Schroeder and Cavacini J Allergy Clin Immunol. (2010) 125(202):S41-S52, which is hereby incorporated by reference in its entirety. Immunoglobulins of type G (i.e. IgG) are ~150 kDa glycoproteins comprising two heavy chains and two light chains. From N- to C-terminus, immunoglobulin heavy chains comprise a heavy chain variable region (VH) followed by a heavy chain constant region comprising three constant regions (CH1, CH2, and CH3, with a CH1-CH2 hinge region provided between CH1 and CH2). Immunoglobulin light chains comprise a light chain variable region (VL) followed by a light chain constant region (CL). Depending on the heavy chain, immunoglobulins may be classed as IgG (e.g. IgG1, IgG2, IgG3, IgG4), IgA (e.g. IgA1, IgA2), IgD, IgE, or IgM. The light chain may be kappa (κ) or lambda (λ).

In some embodiments, the polypeptide is conjugated, joined, or fused to another component (e.g. an immunoglobulin) by a linker.

Linker sequences are known to the skilled person, and are described, for example in Chen et al., Adv Drug Deliv Rev. (2013) 65(10):1357-1369, which is hereby incorporated by reference in its entirety. In some embodiments, a linker sequence may be a flexible linker sequence. Flexible linker sequences allow for relative movement of the amino acid sequences which are linked by the linker sequence. Flexible linkers are known to the skilled person, and several are identified in Chen et al., Adv Drug Deliv Rev. (2013) 65(10):1357-1369. Flexible linker sequences often comprise high proportions of glycine and/or serine residues. In some embodiments, a linker sequence comprises at least one glycine residue and/or at least one serine residue. In some embodiments, the linker sequence comprises or consists of glycine and serine residues. In some embodiments, the polypeptide is conjugated, joined, or fused to another component (e.g. an immunoglobulin) by a linker with the amino acid sequence EPKSA (SEQ ID NO:27), or an amino acid sequence comprising up to three modifications (e.g. substitutions, insertions, or deletions) relative to SEQ ID NO:27.

In some embodiments, the polypeptide derived from RetroNectin® (SEQ ID NO:1) or vitronectin (SEQ ID NO:2 or SEQ ID NO:3) has the same functionality as the polypeptide that it is derived from. In some embodiments, the polypeptide derived from RetroNectin® (SEQ ID NO:1) or vitronectin (SEQ ID NO:2 or SEQ ID NO:3) has improved functionality compared with the polypeptide that it is derived from.

In some embodiments, the polypeptide derived from RetroNectin® (SEQ ID NO:1) or vitronectin (SEQ ID NO:2 or SEQ ID NO:3) possess one or more of the following properties:
Increases cell adhesion
Increases cell adhesion to a surface
Increases cell adhesion to a culture vessel
Increases cell adhesion to an inner surface of a culture vessel.
Increases cell adhesion compared to the cell adhesion of control cells cultured in the same way but without the polypeptide, as assessed by microscopy, a flow chamber pressure assay, a non-invasive and label-free real-time impedance assay, or through an assay utilising anti-cell adhesion antibodies.
Increases cell adhesion by 1.5 times compared to the cell adhesion of control cells cultured in the same way but without the polypeptide Increases cell adhesion by one of 2 times, 3 times, 4 times, or 5 times compared to the cell adhesion of control cells cultured in the same way but without the polypeptide
Increases cell adhesion in the absence of fetal bovine serum (FBS)
Increases cell adhesion in FBS-free media
Increases cell adhesion in the absence of serum
Increases cell adhesion in serum-free media
Increases cell adhesion in the absence of animal products
Increases cell adhesion in animal-product-free media
Increases cell adhesion in xeno-free media
Reduces cellular aggregation
Reduces cellular aggregation compared to the aggregation of control cells cultured in the same way but without the polypeptide, as assessed by microscopy.
Reduces cellular aggregation by 1.5 times compared to the aggregation of control cells cultured in the same way but without the polypeptide.
Reduces cellular aggregation by 2 times compared to the aggregation of control cells cultured in the same way but without the polypeptide
Increases viral titre
Increases viral titre in culture supernatant
Increases viral titre in the culture supernatant after transfection
Increases viral titre in the culture supernatant after transfection with a plasmid containing a gene of interest
Increases viral titre in the culture supernatant after 6 hours of post-transfection incubation
Increases viral titre in the culture supernatant after 12 hours of post-transfection incubation
Increases viral titre in the culture supernatant after 24 hours of post-transfection incubation
Increases viral titre in the culture supernatant after 48 hours of post-transfection incubation
Increases viral titre in the culture supernatant after 72 hours of post-transfection incubation
Increases viral titre when the polypeptide is used compared to the viral titre produced by control cells cultured in the same way but without the polypeptide.
Increases viral titre by over 1.5 times when the polypeptide is used compared to the viral titre produced by control cells cultured in the same way but without the polypeptide.
Increases viral titre by one of over 2 times, 3 times, 4 times, 5 times, 6 times, or 7 times when the polypeptide is used compared to the viral titre produced by control cells cultured in the same way but without the polypeptide.
Increases/enhances viral-mediated gene transduction
Increases/enhances viral-mediated gene transduction of mammalian cells
Increases transduction efficiency compared with transduction without the use of the polypeptide
Increases transduction efficiency compared with transduction without the use of a transduction enhancer
Improves transduction efficiency by one of at least 1.75, 2.0, 2.25, 2.5, 2.75, 3.0, 3.25, 3.5, 3.75, or 4.0 times compared with transduction without the use of a transduction enhancer
Improves transduction efficiency by one of at least 1.1, 1.2, 1.3, 1.4, 1.5, 1.6, 1.7, 1.8, 1.9, 2.0, 2.25, 2.5, 2.75, 3.0, 3.25, 3.5, 3.75, or 4.0 times compared with transduction without the use of a transduction enhancer
Improves transduction efficiency by one of at least 1.1 times compared with transduction without the use of a transduction enhancer
Improves transduction efficiency by one of at least 1.75, 2.0, 2.25, 2.5, 2.75, 3.0, 3.25, 3.5, 3.75, or 4.0 times compared with transduction without the use of the polypeptide
Improves transduction efficiency by one of at least 1.1, 1.2, 1.3, 1.4, 1.5, 1.6, 1.7, 1.8, 1.9, 2.0, 2.25, 2.5, 2.75, 3.0, 3.25, 3.5, 3.75, or 4.0 times compared with transduction without the use of the polypeptide
Improves transduction efficiency by one of at least 1.1 times compared with transduction without the use of the polypeptide
Reduces cytokine secretion from cells transduced with a chimeric antigen receptor (CAR)
Reduces cytokine secretion from cells for use in adoptive cell transfer
Reduces cytokine secretion from CAR-T cells
Improves the cell killing properties of cells transduced with a CAR
Improves the cell killing properties of cells for use in adoptive cell transfer
Improves the cell killing properties of CAR T cells

### RetroNectin® and Vitronectin as Adhesion Enhancers

Inventors have demonstrated that RetroNectin® and vitronectin can enhance the adhesion of cells to a culture vessel when cells are grown in an adherent culture. Additionally, it has been shown that RetroNectin® and vitronectin can improve the production of viral vectors in the absence of FBS in serum-free media. The improved adhesion led to increased viral vector production and increased yields of viral vectors as demonstrated in the Examples.

It has been shown that this improvement in adhesion and increased viral vector production can be achieves in multiple ways.

RetroNectin®, vitronectin, polypeptides derived from RetroNectin®, and/or polypeptides derived from vitronectin can be present in the media, added to the media, and/or used as a coating to the culture vessel.

### RetroNectin® and Vitronectin in Media

Most adherent processes are dependent on products of animal origin such as fetal bovine serum (FBS) that pose safety, consistency and regulatory challenges. In fact, most media which is used to culture adherent cells contains FBS and/or other serum components within the media. Inventors have shown that RetroNectin® and vitronectin can be used in media instead of serum components such as FBS to enable efficient adhesion of cells to culture vessels.

The RetroNectin®, vitronectin, polypeptides derived from RetroNectin®, and/or polypeptides derived from vitronectin can be present in the media at the start of the process, or can be added to the media during the culture process, for example RetroNectin®, vitronectin, polypeptides derived from RetroNectin®, and/or polypeptides derived from vitronectin may be added to the media during cell seeding. Therefore, RetroNectin®, vitronectin, polypeptides derived from RetroNectin®, and/or polypeptides derived from vitronectin may be considered as a media component or a media additive.

RetroNectin®, vitronectin, polypeptides derived from RetroNectin®, and/or polypeptides derived from vitronectin may be added to media in a number of formats. For example, RetroNectin®, vitronectin, polypeptides derived from RetroNectin®, and/or polypeptides derived from vitronectin may be added to the media as a liquid/aqueous solution, or as a powder or gel.

RetroNectin®, vitronectin, polypeptides derived from RetroNectin®, and/or polypeptides derived from vitronectin may be added to the media in the form that it was provided from a manufacturer, for example as a sterile 1 mg/mL solution containing 12.5 mM sodium citrate (pH 6.2) and 1.25% sucrose.

RetroNectin®, vitronectin, polypeptides derived from RetroNectin®, and/or polypeptides derived from vitronectin may be added to the media from a diluted stock solution. RetroNectin®, vitronectin, polypeptides derived from RetroNectin®, and/or polypeptides derived from vitronectin may be added to the media as one of a 0.001, 0.005, 0.01, 0.05, 0.1, 0.2, 0.3, 0.4, 0.5, 0.6, 0.7, 0.8, or 0.9 mg/mL solution.

RetroNectin®, vitronectin, polypeptides derived from RetroNectin®, and/or polypeptides derived from vitronectin may be added to the media at a concentration of 20 μg/mL within the media. RetroNectin®, vitronectin, polypeptides derived from RetroNectin®, and/or polypeptides derived from vitronectin may be added to the media at a concentration of 0.1, 0.5, 1, 2, 3, 4, 5, 6, 7, 8, 9, 10, 11, 12, 13, 14, 15, 16, 17, 18, 19, or 20 μg/mL within the media.

RetroNectin®, vitronectin, polypeptides derived from RetroNectin®, and/or polypeptides derived from vitronectin may be added to the media at a concentration of over 20 μg/mL within the media. RetroNectin®, vitronectin, polypeptides derived from RetroNectin®, and/or polypeptides derived from vitronectin may be added to the media at a concentration of over 0.1, 0.5, 1, 2, 3, 4, 5, 6, 7, 8, 9, 10, 11, 12, 13, 14, 15, 16, 17, 18, 19, or 20 μg/mL within the media.

RetroNectin®, vitronectin, polypeptides derived from RetroNectin®, and/or polypeptides derived from vitronectin may be added to the media at a concentration of less than 20 μg/mL within the media. RetroNectin®, vitronectin, polypeptides derived from RetroNectin®, and/or polypeptides derived from vitronectin may be added to the media at a concentration of less than 0.1, 0.5, 1, 2, 3, 4, 5, 6, 7, 8, 9, 10, 11, 12, 13, 14, 15, 16, 17, 18, 19, or 20 μg/mL within the media.

### RetroNectin® and Vitronectin Coatings

As described above, most adherent cell culture processes are dependent on products of animal origin such as fetal bovine serum (FBS) that pose safety, consistency, and regulatory challenges. Inventors have shown that RetroNectin® and vitronectin can be coated on culture vessels to prevent the need to use serum components such as FBS to enable efficient adhesion of cells to culture vessels.

As shown in the examples, RetroNectin® and vitronectin can be coated on culture vessels to increase adhesion, viral production, and viral yield, even in the absence of serum components such as FBS.

The skilled person is aware of many ways in which RetroNectin®, vitronectin, polypeptides derived from RetroNectin®, and/or polypeptides derived from vitronectin may be coated on culture vessels. RetroNectin®, vitronectin, polypeptides derived from RetroNectin®, and/or polypeptides derived from vitronectin may be coated only on the interior surface of the culture vessel, or the whole culture vessel may be coated with RetroNectin®, vitronectin, polypeptides derived from RetroNectin®, and/or polypeptides derived from vitronectin.

The examples demonstrate exemplary methods of coating culture vessels with RetroNectin® and/or vitronectin. Other methods are possible.

RetroNectin® and/or vitronectin, or polypeptides derived from RetroNectin® and/or vitronectin, may be diluted to a range of concentrations in PBS (e.g. 0.1, 1, 4, 8 and 16 μg/mL in a solvent such as PBS). An appropriate volume of the solution can be added to the culture vessel, before incubating overnight at 4° C. Studies have also shown that leaving the coating at room temperature, or 2-3 hours in the incubator also promotes cell adherence after applying the RetroNectin®. The appropriate concentration, volume of solution, incubation time, and incubation temperature may be adjusted by the skilled person.

In some embodiments, the incubation is for at least 1, 2, 3, 4, 5, 6, 7, 8, 9, 10, 11, 12, 13, 14, 15, 16, 17, 18, 19, 20, 21, 22, 23, or 24 hour(s). In some embodiments, the incubation is for at least 1, 2, 3, 4, 5, 6, 7, 8, 9, 10, 11, 12, 13, 14, 15, 16, 17, 18, 19, 20, 21, 22, 23, 24, 25, 26, 27, or 28 day(s).

In some embodiments, the incubation is performed in a refrigerator, at room temperature, or in an incubator at ≥30° C.

In some embodiments, the solution comprising the polypeptide contains the polypeptide at a concentration of at least 0.1 μg/mL. In some embodiments, the solution comprising the polypeptide contains the polypeptide at a concentration of at least 0.5 μg/mL, 1 μg/mL, 2 μg/mL, 3 μg/mL, 4 μg/mL, 5 μg/mL, 10 μg/mL, 15 μg/mL, or 20 μg/mL.

In some embodiments, the culture vessel is a culture plate, a culture dish, a culture flask, a culture bottle or a bioreactor. In some embodiments, the culture plate is a 2-well plate, 4-well plate, a 6-well plate, a 12-well plate, a 24-well plate, a 48-well plate, a 96-well plate, or a 384 well plate.

In some embodiments, the culture vessel has a volume of at least 0.2 mL. In some embodiments, the culture vessel has a volume of at least 0.5 mL, preferably wherein the culture vessel has a volume of at least 0.6, 0.7, 0.8, 0.9, 1.5, 1.0, 2.0, 3.0, 4.0, 5.0, 10.0, 15.0, 20.0, 25.0, 30.0, 35.0, 40.0, 45.0, 50.0, 60.0, 70.0, 80.0, 90.0, 100.0, 125.0, 150.0, 175.0, or 200.0 mL.

In some embodiments, culture vessels are coated with RetroNectin® and/or vitronectin, or polypeptides derived from RetroNectin® and/or vitronectin. One or several layers of a coating may be applied to a culture vessel. The coating is a substrate for cell growth and may increase adherence of cells to the culture vessel.

The culture vessel may be coated with a matrix, which in the context of this document refers to a layer (e.g. a thin layer or film) of substance attached to the culture vessel such as on its surface. The matrix may comprise RetroNectin and/or vitronectin, and/or polypeptides derived from RetroNectin® and/or vitronectin. The matrix may comprise a further compatible or physiologically relevant matrix capable of supporting growth of cells.

The matrix may comprise a further component of the extracellular matrix (ECM). Any of the known components of the ECM such as those capable of supporting growth of cells may be used. Components of the extracellular matrix are known in the art and are described in for example Alberts et al., Molecular Biology of the Cell, (2002) Chapter IV and references cited therein.

In some embodiments, the matrix is fetal bovine serum (FBS)-free, serum-free, xeno-free, or animal product-free.

Additional matrix materials may include poly-L-lysine, laminin, gelatin, collagen, keratin, fibronectin, elastin, heparan sulphate, dextran, dextran sulphate, chondroitin sulphate or a mixture of laminin, collagen I, heparan sulfate proteoglycans, and entactin 1, and derivatives or fragment thereof. The ECM component may be attached or coupled to or coated on the culture vessel through conventional means. For example, any of the coupling reagents and crosslinkers described above may be used to couple the ECM component to the culture vessel.

The additional ECM component may comprise a macromolecule such as a polysaccharide, protein, proteoglycan, glycoprotein, glycosaminoglycan (GAG), usually found covalently linked to protein in the form of proteoglycans, a fibrous protein, including elastin, fibronectin, and laminin, collagen (e.g. collagen I, collagen III, collagen IV, collagen VI) etc.

The matrix coating may comprise a glycosaminoglycan (GAG). Glycosaminoglycans comprise unbranched polysaccharide chains composed of repeating disaccharide units. One of the two sugars in the repeating disaccharide is always an amino sugar (N-acetylglucosamine or N-acetylgalactosamine), which in most cases is sulfated. The second sugar is usually a uronic acid (glucuronic or iduronic). The matrix coating may comprise hyaluronan (also called hyaluronic acid or hyaluronate) or a derivative thereof. The hyaluronic acid may be derived from any number of sources, such as from bovine vitreous humor. A salt or base of hyaluronic acid may be employed, such as hyaluronic acid sodium. This may be from streptococcus.

The matrix coating may comprise laminin and/or fibronectin and/or collagen and/or keratin and/or gelatin.

The matrix coating may comprise for example a GAG such as chondroitin sulfate, dermatan sulfate, heparan sulfate and keratan sulfate, for example as linked to a protein as a proteoglycan. The matrix coating may comprise aggrecan, decorin, etc.

The matrix coating may comprise heparan or its derivatives such as bases or salts. The matrix coating may comprise heparan sulphate proteoglycan. The heparan sulphate proteoglycan may be derived from any number of sources, such as from bovine kidney.

The matrix coating may comprise a dextran such as dextran sulphate or dextran sulphate sodium. The matrix coating may comprise fibronectin, laminin, nidogen or Type IV collagen. The matrix coating may comprise chondroitin sulphate.

The matrix may comprise gelatin, polyomithine, or binding motifs of the RGD binding domain of fibronectin.

ECM matrix materials include: laminin, fibronectin, vitronectin, collagen, elastin, aggrecan, decorin, chondroitin sulfate, dermatan sulfate, heparan sulphate, keratan sulphate, and hyaluronan.

The matrix coating may comprise a mixture of any two or more of the components described above in various proportions. The matrix coating may comprise a purified or substantially purified component, e.g. of the ECM. The matrix component may comprise a partially purified component of the ECM. It may comprise an ECM extract such as Matrigel™.

### RetroNectin® and Vitronectin as Transduction Enhancers

Aspects of the current invention involve viral mediated gene transduction. Transduction is the process by which foreign DNA is introduced into a cell by a virus or viral vector (Bouard et al. Br J Pharmacol. (2009) 157(2):153-165). Transduction does not require physical contact between the cell donating the DNA and the cell receiving the DNA (which occurs in conjugation), and it is DNase resistant (transformation is susceptible to DNase). Transduction is a common tool used by molecular biologists to stably introduce a foreign gene into a host cell's genome (both bacterial and mammalian cells).

Transduction with viral vectors can be used to insert or modify genes in mammalian cells. A plasmid is constructed in which the genes to be transferred are flanked by viral sequences that are used by viral proteins to recognize and package the viral genome into viral particles. This plasmid is inserted (usually by transfection) into a producer cell together with other plasmids (DNA constructs) that carry the viral genes required for the formation of infectious virions. In these producer cells, the viral proteins expressed by these packaging constructs bind the sequences on the DNA/RNA (depending on the type of viral vector) to be transferred and insert it into viral particles. Viruses collected from these cells are then applied to the cells to be altered. This leads to expression of the genes transferred and (in the case of lentivirus/retrovirus vectors) insertion of the DNA to be transferred into the cellular genome.

To make cell modification as effective as possible, it is important to ensure that transduction is efficient. To achieve this aim, it is possible to use additives to enhance transduction. Inventors have shown that RetroNectin®, vitronectin, polypeptides derived from RetroNectin®, and polypeptides derived from vitronectin function as transduction enhancers.

It has been shown that this improvement in transduction can be achieved in multiple ways. RetroNectin® and/or vitronectin (or polypeptides derived from RetroNectin® and/or vitronectin) can be present in the media, added to the media, or used as a coating to the culture vessel. Details as to how RetroNectin®, vitronectin, polypeptides derived from RetroNectin®, and polypeptides derived from vitronectin can be present in the media, added to the media, or used as a coating to the culture vessel are presented in previous sections.

Transduction efficiency can be determined through any method known in the art. One such method is shown in the examples (e.g. Example 3), in which a flow cytometry assay is utilised to determine the percentage of cells which have been transduced. The percentage of cells that are transduced indicated the transduction efficiency. The skilled person would be aware of numerous assays to determine transduction efficiency.

By way of example, if cells are transduced with CV-hCD19 LV vectors, and 25% of cells later express a CD19 CAR on the cell surface, then the transduction efficiency is 25%.

By way of example, if the transduction efficiency of a group which did not use a transduction enhancer was 25%, and the transduction efficiency of a group which did use a polypeptide as a transduction enhancer was 27.5%, then the polypeptide improves transduction efficiency—in fact, the polypeptide improves transduction efficiency by 1.1 times (i.e. 10% improvement), and the transduction efficiency for the group that used the polypeptide as a transduction enhancer is 1.1 times higher than the transduction efficiency of the group which did not use a transduction enhancer.

In some embodiments, the polypeptide improves transduction efficiency compared to the transduction efficiency without the use of a transduction enhancer.

In some embodiments, the polypeptide improves transduction efficiency by at least 1.1 times compared to transduction efficiency without the use of a transduction enhancer. In some embodiments, the polypeptide improves transduction efficiency by at least 1.2 times compared to the transduction efficiency without the use of a transduction enhancer, preferably wherein the polypeptide improves transduction efficiency by at least 1.3, 1.4, 1.5, 1.75, 2.0, 2.25, 2.5, 2.75, 3.0, 3.25, 3.5, 3.75, or 4.0 times compared to transduction efficiency without the use of a transduction enhancer.

In some embodiments, the transduction efficiency is at least 1.1 times higher than transduction efficiency without the use of a transduction enhancer. In some embodiments, the transduction efficiency is 1.2, 1.3, 1.4, 1.5, 1.75, 2.0, 2.25, 2.5, 2.75, 3.0, 3.25, 3.5, 3.75, or 4.0 times higher than transduction efficiency without the use of a transduction enhancer.

### Inhibitors of Cytokine Secretion

Cytokines such as IL-6 are known as central mediators of Cytokine Release Syndrome (CRS) toxicity associated with CAR-T cell therapy (Tanaka et al. Immunotherapy (2016) 8(8):959-70). A challenge in the development of cell therapies (e.g., CAR-T cell therapies) is the need to reduce the impact of cytokine release syndrome without reducing the therapeutic efficacy of the treatment.

The examples show that polypeptides of this disclosure are capable of inhibiting cytokine secretion of cells.

The examples also show that polypeptides of this disclosure are capable of inhibiting cytokine secretion of cells, without having a negative effect on the cell killing properties/cytotoxicity of the cells.

In some embodiments, polypeptides described in this disclosure are capable of inhibiting cytokine secretion from cells. In some embodiments, polypeptides described in this disclosure are capable of inhibiting cytokine secretion from immune cells. In some embodiments, polypeptides described in this disclosure are capable of inhibiting cytokine secretion from effector immune cells. In some embodiments, polypeptides described in this disclosure are capable of inhibiting cytokine secretion from cells suitable for use in adoptive cell transfer. In some embodiments, polypeptides described in this disclosure are capable of inhibiting cytokine secretion from cells used in adoptive cell transfer. In some embodiments, polypeptides described in this disclosure are capable of inhibiting cytokine secretion from cells used in cell therapy. In some embodiments, polypeptides described in this disclosure are capable of inhibiting cytokine secretion from T cells or NK cells. In some embodiments, polypeptides described in this disclosure are capable of inhibiting cytokine secretion from CAR T cells or CAR NK cells.

In some embodiments, polypeptides described in this disclosure are capable of inhibiting cytokine secretion from cells whilst maintaining the cell killing properties of the cells. In some embodiments, polypeptides described in this disclosure are capable of inhibiting cytokine secretion from immune cells whilst maintaining the cell killing properties of the immune cells. In some embodiments, polypeptides described in this disclosure are capable of inhibiting cytokine secretion from effector immune cells whilst maintaining the cell killing properties of the effector immune cells. In some embodiments, polypeptides described in this disclosure are capable of inhibiting cytokine secretion from cells suitable for use in adoptive cell transfer whilst maintaining the cell killing properties of the cells suitable for use in adoptive cell transfer. In some embodiments, polypeptides described in this disclosure are capable of inhibiting cytokine secretion from cells used in adoptive cell transfer whilst maintaining the cell killing properties of the cells used in adoptive cell transfer. In some embodiments, polypeptides described in this disclosure are capable of inhibiting cytokine secretion from cells used in cell therapy whilst maintaining the cell killing properties of the cells used in cell therapy. In some embodiments, polypeptides described in this disclosure are capable of inhibiting cytokine secretion from T cells or NK cells whilst maintaining the cell killing properties of the T cells or NK cells. In some embodiments, polypeptides described in this disclosure are capable of inhibiting cytokine secretion from CAR T cells or CAR NK cells whilst maintaining the cell killing properties of the CAR T cells or CAR NK cells.

In some embodiments, polypeptides described in this disclosure are capable of inhibiting cytokine secretion from cells without inhibiting the killing of target cells. In some embodiments, polypeptides described in this disclosure are capable of inhibiting cytokine secretion from immune cells without inhibiting the killing of target cells. In some embodiments, polypeptides described in this disclosure are capable of inhibiting cytokine secretion from effector immune cells without inhibiting the killing of target cells. In some embodiments, polypeptides described in this disclosure are capable of inhibiting cytokine secretion from cells suitable for use in adoptive cell transfer without inhibiting the killing of target cells. In some embodiments, polypeptides described in this disclosure are capable of inhibiting cytokine secretion from cells used in adoptive cell transfer without inhibiting the killing of target cells. In some embodiments, polypeptides described in this disclosure are capable of inhibiting cytokine secretion from cells used in cell therapy without inhibiting the killing of target cells. In some embodiments, polypeptides described in this disclosure are capable of inhibiting cytokine secretion from T cells or NK cells without inhibiting the killing of target cells. In some embodiments, polypeptides described in this disclosure are capable of inhibiting cytokine secretion from CAR T cells or CAR NK cells without inhibiting the killing of target cells.

In one aspect, a polypeptide comprising an amino acid sequence having at least 70% sequence identity to one of SEQ ID NO:3, SEQ ID NO:2, or SEQ ID NO:1, or a polypeptide comprising an amino acid sequence having at least 70% sequence identity to a polypeptide derived from SEQ ID NO:3, SEQ ID NO:2, or SEQ ID NO:1 is contacted with a cell.

In some embodiments, a polypeptide comprising an amino acid sequence having at least 70% sequence identity to one of SEQ ID NO:3 or SEQ ID NO:2, or a polypeptide comprising an amino acid sequence having at least 70% sequence identity to a polypeptide derived from SEQ ID NO:3 or SEQ ID NO:2 is contacted with a cell.

In one aspect, a method of inhibiting and/or reducing cytokine secretion from a cell is provided, wherein the method comprises contacting the cell with a polypeptide comprising an amino acid sequence having at least 70% sequence identity to one of SEQ ID NO:3, SEQ ID NO:2 or SEQ ID NO:1, or a polypeptide comprising an amino acid sequence having at least 70% sequence identity to a fragment of SEQ ID NO:3, SEQ ID NO:2, or SEQ ID NO:1.

In some embodiments, a method of inhibiting and/or reducing cytokine secretion from a cell is provided, wherein the method comprises contacting the cell with a polypeptide comprising an amino acid sequence having at least 70% sequence identity to one of SEQ ID NO:3 or SEQ ID NO:2, or a polypeptide comprising an amino acid sequence having at least 70% sequence identity to a polypeptide fragment of SEQ ID NO:3 or SEQ ID NO:2.

Use of a polypeptide comprising an amino acid sequence having at least 70% sequence identity to one of SEQ ID NO:3, SEQ ID NO:2 or SEQ ID NO:1, or a polypeptide comprising an amino acid sequence having at least 70% sequence identity to a fragment of SEQ ID NO:3, SEQ ID NO:2, or SEQ ID NO:1 in a method of inhibiting and/or reducing cytokine secretion is also envisaged.

Use of a polypeptide comprising an amino acid sequence having at least 70% sequence identity to one of SEQ ID NO:3 or SEQ ID NO:2, or a polypeptide comprising an amino acid sequence having at least 70% sequence identity to a fragment of SEQ ID NO:3 or SEQ ID NO:2 in a method of inhibiting and/or reducing cytokine secretion is also envisaged.

In some embodiments, the cell is a cell for adoptive cell transfer. In some embodiments, the cell is an immune cell. In some embodiments, the cell is an effector immune cell. In some embodiments, the cell is a T cell or an NK cell. In some embodiments, the cell is a CAR T cell or a CAR NK cell.

In some embodiments, the cell killing properties of the cells are not reduced by the method of inhibiting and/or reducing cytokine secretion. In some embodiments, the killing of target cells is not reduced/inhibited. In some embodiments, cytotoxicity of the cells is not reduced/inhibited. In some embodiments, cytotoxicity of the cells is maintained.

In some embodiments, a method of inhibiting and/or reducing cytokine secretion from a cell, whilst maintaining the cytotoxicity of the cell, is provided, wherein the method comprises contacting the cell with a polypeptide comprising an amino acid sequence having at least 70% sequence identity to one of SEQ ID NO:3 or SEQ ID NO:2, or a polypeptide comprising an amino acid sequence having at least 70% sequence identity to a polypeptide fragment of SEQ ID NO:3 or SEQ ID NO:2.

In some embodiments, the cytokine is one or more of IL-6, TNF-α, IFN-γ, and IL-8. In some embodiments, the cytokine is IL-6. In some embodiments, the cytokine is TNF-α. In some embodiments, the cytokine is IFN-γ. In some embodiments, the cytokine is IL-8.

In some embodiments, the method is performed in vitro, ex vivo, or in vivo. In some embodiments, the method is an in vitro method. In some embodiments, the method is an ex vivo method. In some embodiments, the method is an in vivo method.

In some embodiments, the cell is contacted with the polypeptide in vitro, ex vivo, or in vivo. In some embodiments, the cell is contacted with the polypeptide in vitro. In some embodiments, the cell is contacted with the polypeptide ex vivo. In some embodiments, the cell is contacted with the polypeptide in vivo.

The level of cytokine secretion by a cell may be compared to an appropriate control. For example, the level of cytokine secretion by a cell that has been contacted with a polypeptide according to the present disclosure may be compared to a cell that is not contacted with a polypeptide according to this disclosure.

Alternatively, the level of cytokine secretion by a cell that has been contacted with a polypeptide according to the present disclosure may be compared to a cell that is contacted with a known transduction enhancer. Alternatively, the level of cytokine secretion by a cell that has been contacted with a polypeptide according to the present disclosure may be compared to a cell that is contacted with RetroNectin®.

The level of cytokine secretion by a cell may be assessed through any method known in the art, for example through the methods used in Example 9 of the present disclosure. In such an experiment, a cell could be incubated for a period of time (e.g. 24 hours) in a suitable medium, before analysis of supernatant is performed. Many suitable methods of supernatant analysis are known to the skilled person, e.g. ELISA, mass spectrometry, or commercial cytokine assay kits. In the experiment of Example 9, CAR expressing T-cells from each of the conditions were co-cultured with NALM-6-Luc tumor cells expressing Luciferase at either 4- or 14-days post-transduction. After 24 hours of co-culture, cell culture supernatants were collected. An automated ELISA (ELLA Immunoassay system, ProteinSimple, USA) was performed on cell culture supernatant samples to measure cytokine levels between transduction enhancer treated or control groups. This experiment could be easily adapted by a skilled person to assess the level of cytokine secretion by many different cell types. The skilled person would be aware of many ways to assess the cytokine secretion levels of cells.

### Producing a Cell for Adoptive Cell Therapy

Methods disclosed herein may involve the stimulation and/or expansion of immune cells. The methods may involve the stimulation and/or expansion of peripheral blood cells, such as peripheral blood mononuclear cells. The methods may involve expansion of an immune cell population (e.g. a population of T-cells) from within a population of immune cells (e.g. PBMCs). For example, a population of T-cells may be expanded from within a population of PBMCs, by stimulation of the T-cells within the population of PBMCs. Accordingly, in embodiments of the methods disclosed herein, stimulation and/or expansion of T cells may involve stimulation of a population of PBMCs. In some embodiments, a population of T-cells may be expanded from within a population of tumor-infiltrating lymphocytes, by stimulation of the T-cells within the population of tumor-infiltrating lymphocytes. Accordingly, in embodiments of the methods disclosed herein, stimulation and/or expansion of T cells may involve stimulation of a population of tumor-infiltrating lymphocytes. In some embodiments, a population of T-cells may be expanded from within a population of T-cells (e.g. a population of T cells of heterogeneous specificity), which may have been obtained from a blood sample, a population of PBMCs, or from a population of tumor-infiltrating lymphocytes. The methods may involve the stimulation and/or expansion of T cells. The cells may have been obtained from the patient to be treated (i.e., autologous cells), or from another individual (i.e., allogeneic cells). The methods involve stimulation and/or expansion of isolated immune cells, in certain embodiments. That is, specific methods may be performed on a population of cells that contains substantially no non-immune cells, such as erythrocytes. In some cases, the immune cells are isolated PBMCs, or isolated T cells.

The cells may have been obtained from a sample of blood, such as a sample of blood obtained from the patient or individual. The cells may have been obtained from tissue sample or biopsy. The cells may have been obtained from a tumor (e.g. tumor-infiltrating lymphocytes). Certain methods disclosed herein involve a step of obtaining PBMCs and/or T cells from a sample obtained from the patient. Certain embodiments of methods do not involve the step of obtaining a sample of blood or cells from the patient or individual, but instead are performed on a sample or cells that have been previously obtained. The method may involve processing the sample, such as enriching the sample for immune cells, such as PBMCs and/or T cells. Such methods may involve removing or substantially reducing the amount of, erythrocytes, platelets, serum and/or plasma in a sample. This may result in a population of immune cells containing substantially no other cells, such as erythrocytes. Methods disclosed herein may be performed on isolated immune cells, or a sample containing immune cells in addition to other cells.

In some embodiments the generated/expanded populations of immune cells are prepared for use in the subject from which the initial population of immune cells from which the population is generated/expanded was obtained/derived. In some embodiments the generated/expanded populations of immune cells are prepared for use with any subject, e.g. a different subject to the subject from which the initial population of immune cells from which the population is generated/expanded was obtained/derived.

In some embodiments, the cells are modified to express a certain non-natural receptor that allows the T-cells to effectively or more effectively target a desired target cell, such as one that expresses a certain antigen. The cells may be modified to express a chimeric antigen receptor (CAR), a T-cell receptor, and so forth. The cells may be modified to express an expression vector (that may be viral (including retroviral, lentiviral, adenoviral, adeno-associated viral, and so forth) or non-viral) during the method at specific time points, such as the vector being introduced between day 2 and 5 of culture, for example. In some embodiments the cells are exposed to the expression vector within about 3 days after each stimulation, but in such cases the modification occurs in more differentiated T-cells that have less long term potential (which in specific circumstances is desirable).

In specific embodiments, the cells are modified to express a CAR that targets a cancer antigen, such as EphA2, HER2, GD2, Glypican-3, 5T4, 8H9, αvβ6 integrin, B cell maturation antigen (BCMA) B7-H3, B7-H6, CAIX, CA9, CD19, CD20, CD22, kappa light chain, CD30, CD33, CD38, CD44, CD44v6, CD44v7/8, CD70, CD123, CD138, CD171, CEA, CSPG4, EGFR, EGFRvIII, EGP2, EGP40, EPCAM, ERBB3, ERBB4, ErbB3/4, FAP, FAR, FBP, fetal AchR, Folate Receptor a, GD2, GD3, HLA-AI MAGE A1, HLA-A2, IL11Ra, IL13Rα2, KDR, Lambda, Lewis-Y, MCSP, Mesothelin, Muc1, Muc16, NCAM, NKG2D ligands, NY-ESO-1, PRAME, PSCA, PSC1, PSMA, ROR1, Sp17, SURVIVIN, TAG72, TEM1, TEM8, VEGRR2, carcinoembryonic antigen, HMW-MAA, VEGF receptors, and/or other exemplary antigens that are present with in the extracellular matrix of tumors, such as oncofetal variants of fibronectin, tenascin, or necrotic regions of tumors and other tumor-associated antigens or actionable mutations that are identified through genomic analysis and or differential expression studies of tumors, for example.

### Enhancers of Cell Killing

When preparing cells for use in adoptive cell transfer, it is important to maintain the cell killing properties of the therapeutic cells. Inventors have found that polypeptides of this disclosure are capable of enhancing the cell killing properties of cells for use in adoptive cell transfer.

In some embodiments, polypeptides described in this disclosure are capable of enhancing the cell killing properties of cells. In some embodiments, polypeptides described in this disclosure are capable of enhancing the cell killing properties of immune cells. In some embodiments, polypeptides described in this disclosure are capable of enhancing the cell killing properties of effector immune cells. In some embodiments, polypeptides described in this disclosure are capable of enhancing the cell killing properties of cells used in adoptive cell transfer. In some embodiments, polypeptides described in this disclosure are capable of enhancing the cell killing properties of cells used in cell therapy. In some embodiments, polypeptides described in this disclosure are capable of enhancing the cell killing properties of T cells or NK cells. In some embodiments, polypeptides described in this disclosure are capable of enhancing the cell killing properties of CAR T cells or CAR NK cells.

In one aspect, a method of enhancing or increasing killing of target cells by a cell is provided. In some embodiments, the cell is contacted with a polypeptide comprising an amino acid sequence having at least 70% sequence identity to one of SEQ ID NO:3, SEQ ID NO:2 or SEQ ID NO:1, or a polypeptide comprising an amino acid sequence having at least 70% sequence identity to a fragment of SEQ ID NO:3, SEQ ID NO:2, or SEQ ID NO:1.

In some embodiments, the method comprises contacting the cell with a polypeptide comprising an amino acid sequence having at least 70% sequence identity to one of SEQ ID NO:3 or SEQ ID NO:2, or a polypeptide comprising an amino acid sequence having at least 70% sequence identity to a fragment of SEQ ID NO:3 or SEQ ID NO:2.

In some embodiments, the cell is a cell for adoptive cell transfer. In some embodiments, the cell is an immune cell. In some embodiments, the cell is an effector immune cell. In some embodiments, the cell is a T cell or an NK cell. In some embodiments, the cell is a CAR T cell or a CAR NK cell.

In some embodiments, the cell for adoptive cell transfer is contacted with a polypeptide of this disclosure during cell culture or during a method of transduction. In some embodiments, the cell for adoptive cell transfer is contacted with a polypeptide of this disclosure during cell culture. In some embodiments, the cell for adoptive cell transfer is contacted with a polypeptide of this disclosure during a method of transduction.

In some embodiments, the target cell is a cancer cell. In some embodiments, the cell for adoptive cell transfer is a CAR T cell or a CAR NK cell, and the target cell comprises an antigen which is targeted by the CAR expressed by the cell for adoptive cell transfer. In some embodiments, the cell for adoptive cell transfer is a CAR T cell, and the target cell comprises an antigen which is targeted by the CAR expressed by the cell for adoptive cell transfer. By way of example, in some embodiments, the cell for adoptive cell transfer is a CD19 CAR T cell, and the target cell is CD19 positive (i.e. the target cell expresses CD19 which is targeted by the CD19 CAR T cell.

The improvement or enhancement of cell killing may be compared to an appropriate control. For example, the level of target cell killing by a cell for adoptive cell transfer that has been contacted with a polypeptide according to the present disclosure may be compared to a cell that is not contacted with a polypeptide according to this disclosure. Alternatively, the level of target cell killing by a cell for adoptive cell transfer that has been contacted with a polypeptide according to the present disclosure may be compared to a cell that is contacted with a known transduction enhancer. Alternatively, the level of target cell killing by a cell for adoptive cell transfer that has been contacted with a polypeptide according to the present disclosure may be compared to a cell that is contacted with RetroNectin®.

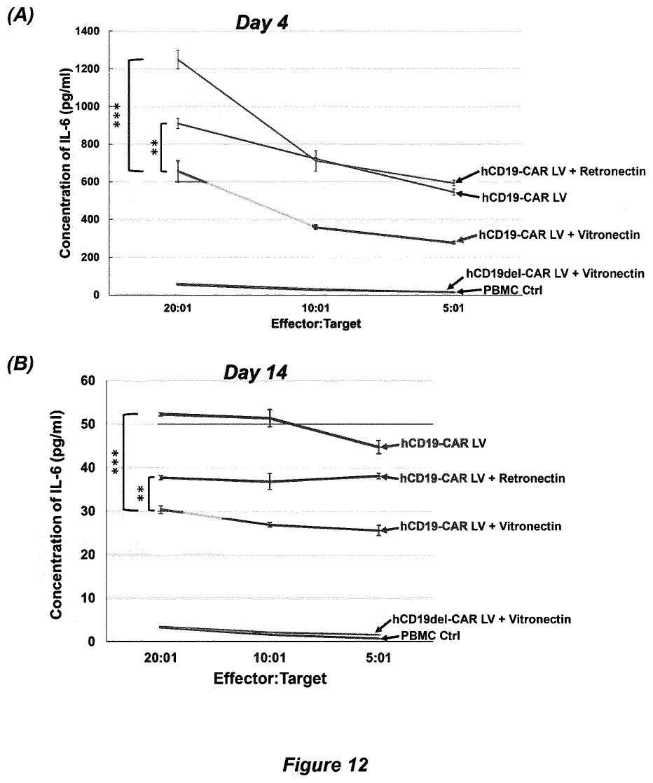

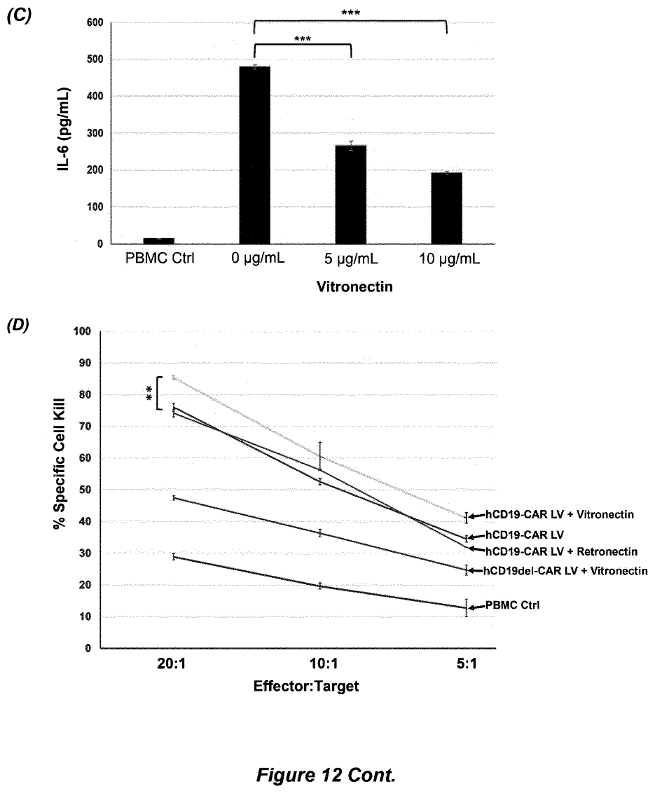

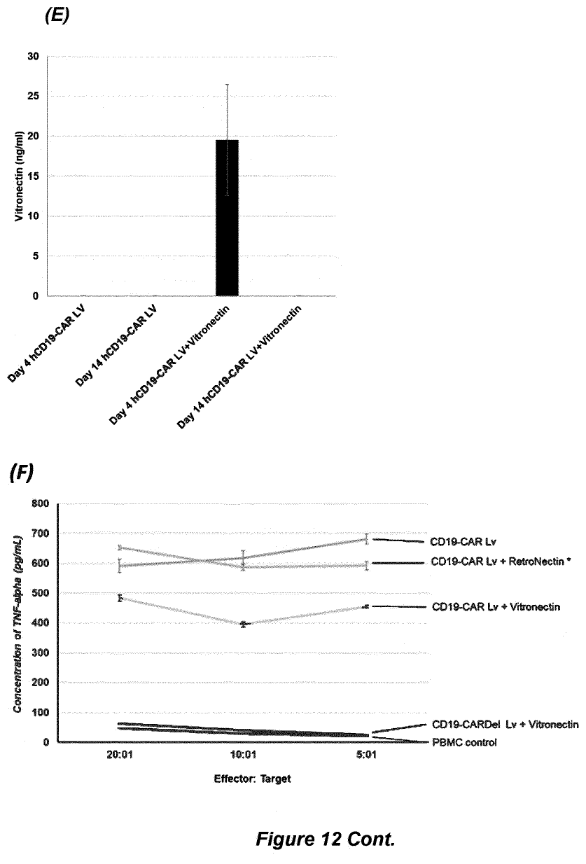

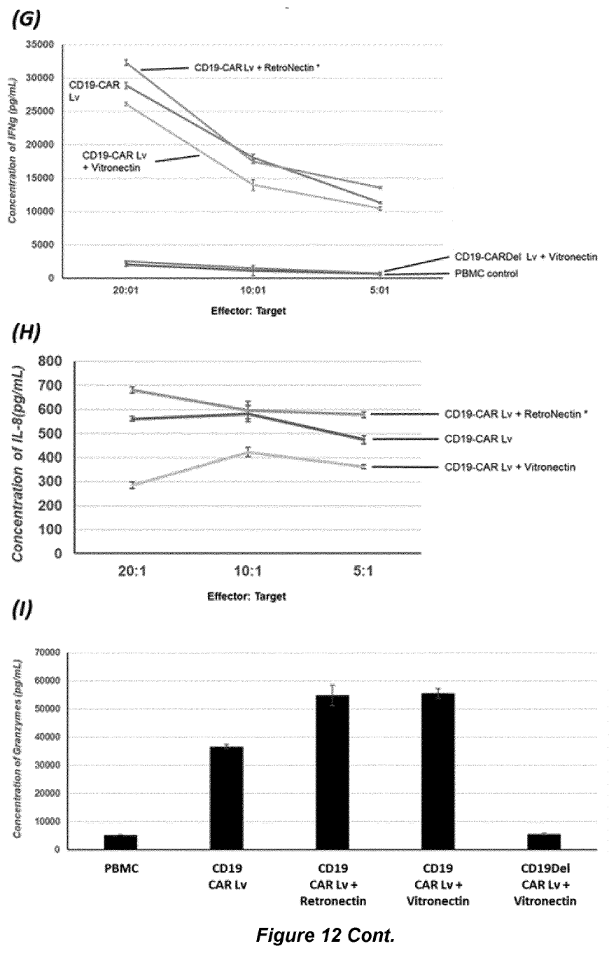

The level of cell killing may be assessed through any method known in the art, for example through the methods used in Example 9 of the present disclosure. In such an experiment, target cells may be modified to express a reporter, and the level or amount of cell killing could be determined through reporter analysis. In Example 9 of the present disclosure, luciferase is used as a reporter. In this experiment, tumor cells expressing Luciferase were separately incubated with CAR T cells from different treatment conditions. Tumour cell kill was measured based on Luciferase activity detected by Bright-Glo™ Luciferase Assay System (Promega, USA). The specific cell kill percentage was calculated from the ratio of total cytolysis to spontaneous cytolysis in each condition. Results are shown in FIG. 12D. It would be understood by the skilled person that other reporter genes could be used, and that different cells suitable for adoptive cell transfer could be assessed in the same way. The skilled person would also be aware of other ways that cell killing levels could be assessed. Techniques such as live microscopic imaging, commercial assays, and other methods of the art could also be sued to assess the level of target cell killing.

### Chimeric Antigen Receptors (CARs)

The present invention relates to cells comprising/expressing chimeric antigen receptors (CARs).

CARs are recombinant receptor molecules which provide both antigen-binding and T cell activating functions. CAR structure and engineering is reviewed, for example, in Dotti et al., Immunol Rev (2014) 257(1), which is hereby incorporated by reference in its entirety.

CARs comprise an antigen-binding domain linked via a transmembrane domain to a signalling domain. An optional hinge domain may provide separation between the antigen-binding domain and transmembrane domain, and may act as a flexible linker. When expressed by a cell, the antigen-binding domain is provided in the extracellular space, and the signalling domain is intracellular.

The antigen-binding domain mediates binding to the target antigen for which the CAR is specific. The antigen-binding domain of a CAR may be based on the antigen-binding region of an antibody which is specific for the antigen to which the CAR is targeted. For example, the antigen-binding domain of a CAR may comprise amino acid sequences for the complementarity-determining regions (CDRs) of an antibody which binds specifically to the target antigen. The antigen-binding domain of a CAR may comprise or consist of the light chain and heavy chain variable region amino acid sequences of an antibody which binds specifically to the target antigen. The antigen-binding domain may be provided as a single chain variable fragment (scFv) comprising the sequences of the light chain and heavy chain variable region amino acid sequences of an antibody. Antigen-binding domains of CARs may target antigen based on other protein:protein interaction, such as ligand:receptor binding; for example an IL-13Rα2-targeted CAR has been developed using an antigen-binding domain based on IL-13 (see e.g. Kahlon et al. Cancer Res (2004) 64(24):9160-9166).

The transmembrane domain is provided between the antigen-binding domain and the signalling domain of the CAR. The transmembrane domain provides for anchoring the CAR to the cell membrane of a cell expressing a CAR, with the antigen-binding domain in the extracellular space, and signalling domain inside the cell. Transmembrane domains of CARs may be derived from transmembrane region sequences for cell membrane-bound proteins (e.g. CD28, CD8, etc.).

The signalling domain comprises amino acid sequences required activation of immune cell function. The CAR signalling domains may comprise the amino acid sequence of the intracellular domain of CD3-ζ, which provides immunoreceptor tyrosine-based activation motifs (ITAMs) for phosphorylation and activation of the CAR-expressing cell. Signalling domains comprising sequences of other ITAM-containing proteins have also been employed in CARs, such as domains comprising the ITAM containing region of FcγRI (Haynes et al., J Immunol 1 (2001) 66(1):182-187). CARs comprising a signalling domain derived from the intracellular domain of CD3-ζ are often referred to as first generation CARs.

The signalling domains of CARs typically also comprise the signalling domain of a costimulatory protein (e.g. CD28, 4-1 BB etc.), for providing the costimulation signal necessary for enhancing immune cell activation and effector function. CARs having a signalling domain including additional co-stimulatory sequences are often referred to as second generation CARs. In some cases CARs are engineered to provide for co-stimulation of different intracellular signalling pathways. For example, CD28 costimulation preferentially activates the phosphatidylinositol 3-kinase (P13K) pathway, whereas 4-1 BB costimulation triggers signalling is through TNF receptor associated factor (TRAF) adaptor proteins. Signalling domains of CARs therefore sometimes contain co-stimulatory sequences derived from signalling domains of more than one co-stimulatory molecule. CARs comprising a signalling domain with multiple co-stimulatory sequences are often referred to as third generation CARs.

An optional hinge region may provide separation between the antigen-binding domain and the transmembrane domain, and may act as a flexible linker. Hinge regions may be flexible domains allowing the binding moiety to orient in different directions, and may e.g. be derived from the CH1-CH2 hinge region of IgG.

Through engineering to express a CAR specific for a particular target antigen, immune cells (typically T cells, but also other immune cells such as NK cells) can be directed to kill cells expressing the target antigen. Binding of a CAR-expressing T cell (CAR-T cell) to the target antigen for which it is specific triggers intracellular signalling, and consequently activation of the T cell. The activated CAR-T cell is stimulated to divide and produce factors resulting in killing of the cell expressing the target antigen.

An “antigen-binding domain” refers to a moiety which is capable of binding to a target antigen. The target antigen may be a peptide/polypeptide, glycoprotein, lipoprotein, glycan, glycolipid, lipid, or fragment thereof. Antigen-binding domains according to the present disclosure may be derived from an antibody/antibody fragment (e.g. Fv, scFv, Fab, single chain Fab (scFab), single domain antibodies (e.g. VhH), etc.) directed against the target antigen, or another target antigen-binding agent (e.g. a target antigen-binding peptide or nucleic acid aptamer, ligand or other molecule).

The CAR of the present disclosure comprises a transmembrane domain. A transmembrane domain refers to any three-dimensional structure formed by a sequence of amino acids which is thermodynamically stable in a biological membrane, e.g. a cell membrane. In connection with the present disclosure, the transmembrane domain may be an amino acid sequence which spans the cell membrane of a cell expressing the CAR.

The transmembrane domain may comprise or consist of a sequence of amino acids which forms a hydrophobic alpha helix or beta-barrel. The amino acid sequence of the transmembrane domain of the CAR of the present invention may be, or may be derived from, the amino acid sequence of a transmembrane domain of a protein comprising a transmembrane domain. Transmembrane domains are recorded in databases such as GenBank, UniProt, Swiss-Prot, TrEMBL, Protein Information Resource, Protein Data Bank, Ensembl, and InterPro, and/or can be identified/predicted e.g. using amino acid sequence analysis tools such as TMHMM (Krogh et al., J Mol Biol (2001) 305:567-580).

The chimeric antigen receptor of the present disclosure comprises a signalling domain. The signalling domain provides sequences for initiating intracellular signalling in cells expressing the CAR.

The signalling domain comprises ITAM-containing sequence. An ITAM-containing sequence comprises one or more immunoreceptor tyrosine-based activation motifs (ITAMs). ITAMs comprise the amino acid sequence YXXL/I (SEQ ID NO:4), wherein “X” denotes any amino acid. In ITAM-containing proteins, sequences according to SEQ ID NO:4 are often separated by 6 to 8 amino acids; YXXL/I(X)6-8YXXL/I (SEQ ID NO:5). When phosphate groups are added to the tyrosine residue of an ITAM by tyrosine kinases, a signalling cascade is initiated within the cell.

In some embodiments, the signalling domain comprises one or more copies of an amino acid sequence according to SEQ ID NO:4 or SEQ ID NO:5. In some embodiments, the signalling domain comprises at least 1, 2, 3, 4, 5 or 6 copies of an amino acid sequence according to SEQ ID NO:4. In some embodiments, the signalling domain comprises at least 1, 2, or 3 copies of an amino acid sequence according to SEQ ID NO:5.

The CAR may further comprise a hinge region. The hinge region may provide between the antigen-binding domain and the transmembrane domain. A hinge region is an amino acid sequence which provides for flexible linkage of the antigen-binding and transmembrane domains of the CAR.

The presence, absence and length of hinge regions has been shown to influence CAR function (reviewed e.g. in Dotti et al., Immunol Rev (2014) 257(1):supra).

In some embodiments the CAR comprises a dimerization domain. As used herein, a “dimerization domain” refers to a sequence of amino acids through which a protein may associate with another protein to form a dimer, oligomer or multimer. In some embodiments the other protein may be a membrane-bound molecule, e.g. a receptor or ligand. In some embodiments, the dimerization domain may provide for self-association of the CAR to form a homodimer, or may provide for association with another, different protein to form a heterodimer.

In some embodiments the CAR comprises one or more linker sequences between the different domains (i.e. the antigen-binding domain, hinge region, transmembrane domain, signalling domain). In some embodiments the CAR comprises one or more linker sequences between subsequences of the domains (e.g. between VH and VL of an antigen-binding domain).

### CAR-Expressing Immune Cells

In some embodiments, CAR-expressing immune cells are envisaged.

The cell may be a eukaryotic cell, e.g. a mammalian cell. The mammal may be a human, or a non-human mammal (e.g. rabbit, guinea pig, rat, mouse or other rodent (including any animal in the order Rodentia), cat, dog, pig, sheep, goat, cattle (including cows, e.g. dairy cows, or any animal in the order Bos), horse (including any animal in the order Equidae), donkey, and non-human primate).

In some embodiments, the cell may be from, or may have been obtained from, a human subject. In some embodiments, the cell may be from, or may have been obtained from blood or an umbilical cord.

The cell may be a cell of hematopoietic origin, e.g. a neutrophil, eosinophil, basophil, lymphocyte, or monocyte. The lymphocyte may be e.g. a T cell, B cell or NK cell or precursor. The cell may express e.g. CD3 polypeptides (e.g. CD3γ CD3ε CD3ζ or CD3δ), TCR polypeptides (TCRα or TCRβ), CD27, CD28, CD4 or CD8.

In some embodiments, the cell is a T cell. In some embodiments, the T cell is a CD3+ T cell. In some embodiments, the T cell is a CD3+, CD8+ T cell. In some embodiments, the T cell is a cytotoxic T cell (e.g. a cytotoxic T lymphocyte (CTL)). In some embodiments, the T-cell is a gamma delta (γδ) T cell, a cytotoxic T cell, or a helper T cell.

In some embodiments, the cell is a target protein-reactive CAR-T cell. In embodiments herein, a “target protein-reactive” CAR-T cell is a cell which displays certain functional properties of a T cell in response to the target protein for which the antigen-binding domain of the CAR is specific, e.g. expressed at the surface of a cell. In some embodiments, the properties are functional properties associated with effector T cells, e.g. cytotoxic T cells.

The present invention also provides a method for producing a cell comprising a nucleic acid or vector according to the present invention, comprising introducing a nucleic acid or vector according to the present invention into a cell. The present invention also provides a method for producing a cell expressing a CAR according to the present invention, comprising introducing a nucleic acid or vector according to the present invention in a cell. In some embodiments, the methods additionally comprise culturing the cell under conditions suitable for expression of the nucleic acid or vector by the cell. In some embodiments, the methods are performed in vitro.

### Viral-Mediated Gene Transduction

In some embodiments, introducing an isolated nucleic acid or vector according to the invention into a cell comprises viral transduction, e.g. lentiviral transduction. Accordingly, in some embodiments the isolated nucleic acid or vector is comprised in a viral vector, or the vector is a viral vector. In some embodiments, the method comprises introducing a nucleic acid or vector according to the invention by electroporation, e.g. as described in Koh et al., Molecular Therapy—Nucleic Acids (2013) 2:e114, which is hereby incorporated by reference in its entirety.

Transduction with viral vectors can be used to insert or modify genes in mammalian cells. A plasmid is constructed in which the genes to be transferred are flanked by viral sequences that are used by viral proteins to recognize and package the viral genome into viral particles. This plasmid is inserted (usually by transfection) into a producer cell together with other plasmids (DNA constructs) that carry the viral genes required for the formation of infectious virions. In these producer cells, the viral proteins expressed by these packaging constructs bind the sequences on the DNA/RNA (depending on the type of viral vector) to be transferred and insert it into viral particles. Viruses collected from these cells are then applied to the cells to be altered. This leads to expression of the genes transferred and (in the case of lentivirus/retrovirus vectors) insertion of the DNA to be transferred into the cellular genome.

The present invention also provides cells obtained or obtainable by the methods for producing a cell according to the present invention.

### Methods of Using Transduced Cells

Transduced cells (e.g. immune cells) described herein (e.g. CAR-expressing T cells such as CD19. CAR T-cells) find use in therapeutic and/or prophylactic methods.

The present disclosure further provides compositions comprising one or more (e.g. a population of CAR-expressing immune cells according to the present disclosure.

The cells described herein may be formulated as pharmaceutical compositions or medicaments for clinical use and may comprise a pharmaceutically acceptable carrier, diluent, excipient or adjuvant. The composition may be formulated for topical, parenteral, systemic, intracavitary, intravenous, intra-arterial, intramuscular, intrathecal, intraocular, intraconjunctival, intratumoral, subcutaneous, intradermal, intrathecal, oral or transdermal routes of administration which may include injection or infusion.

Suitable formulations may comprise the cells in a sterile or isotonic medium. Medicaments and pharmaceutical compositions may be formulated in fluid, including gel, form. Fluid formulations may be formulated for administration by injection or infusion (e.g. via catheter) to a selected region of the human or animal body.

In some embodiments the composition is formulated for injection or infusion, e.g. into a blood vessel or tumor.

The present disclosure also provides methods for the production of pharmaceutically useful compositions, such methods of production may comprise one or more steps selected from: producing a cell described herein; isolating a cell described herein; and/or mixing a cell described herein with a pharmaceutically acceptable carrier, adjuvant, excipient or diluent.

For example, a further aspect the present disclosure relates to a method of formulating or producing a medicament or pharmaceutical composition for use in the treatment of a disease/condition (e.g. a cancer), the method comprising formulating a pharmaceutical composition or medicament by mixing a cell described herein with a pharmaceutically acceptable carrier, adjuvant, excipient or diluent.

A “cancer” can comprise any one or more of the following: acute lymphocytic leukemia (ALL), acute myeloid leukemia (AML), adrenocortical cancer, anal cancer, bladder cancer, blood cancer, bone cancer, brain tumor, breast cancer, cancer of the female genital system, cancer of the male genital system, central nervous system lymphoma, cervical cancer, childhood rhabdomyosarcoma, childhood sarcoma, chronic lymphocytic leukemia (CLL), chronic myeloid leukemia (CML), colon and rectal cancer, colon cancer, endometrial cancer, endometrial sarcoma, esophageal cancer, eye cancer, gallbladder cancer, gastric cancer, gastrointestinal tract cancer, hairy cell leukemia, head and neck cancer, hepatocellular cancer, Hodgkin's disease, hypopharyngeal cancer, Kaposi's sarcoma, kidney cancer, laryngeal cancer, leukemia, leukemia, liver cancer, lung cancer, malignant fibrous histiocytoma, malignant thymoma, melanoma, mesothelioma, multiple myeloma, myeloma, nasal cavity and paranasal sinus cancer, nasopharyngeal cancer, nervous system cancer, neuroblastoma, non-Hodgkin's lymphoma, oral cavity cancer, oropharyngeal cancer, osteosarcoma, ovarian cancer, pancreatic cancer, parathyroid cancer, penile cancer, pharyngeal cancer, pituitary tumor, plasma cell neoplasm, primary CNS lymphoma, prostate cancer, rectal cancer, respiratory system, retinoblastoma, salivary gland cancer, skin cancer, small intestine cancer, soft tissue sarcoma, stomach cancer, stomach cancer, testicular cancer, thyroid cancer, urinary system cancer, uterine sarcoma, vaginal cancer, vascular system, Waldenstrom's macroglobulinemia and Wilms' tumor.

Cancers may be of a particular type. Examples of types of cancer include astrocytoma, carcinoma (e.g. adenocarcinoma, hepatocellular carcinoma, medullary carcinoma, papillary carcinoma, squamous cell carcinoma), glioma, lymphoma, medulloblastoma, melanoma, myeloma, meningioma, neuroblastoma, sarcoma (e.g. angiosarcoma, chrondrosarcoma, osteosarcoma).

The cells, medicament or pharmaceutical composition according to some aspects of the invention may be used in adoptive cell transfer.

Adoptive cell transfer generally refers to a process by which cells (e.g. immune cells) are obtained from a subject, typically by drawing a blood sample from which the cells are isolated. The cells are then typically modified and/or expanded, and then administered either to the same subject (in the case of adoptive transfer of autologous/autogeneic cells) or to a different subject (in the case of adoptive transfer of allogeneic cells). The treatment is typically aimed at providing a population of cells with certain desired characteristics to a subject, or increasing the frequency of such cells with such characteristics in that subject. Adoptive transfer may be performed with the aim of introducing a cell or population of cells into a subject, and/or increasing the frequency of a cell or population of cells in a subject.

Adoptive transfer of immune cells is described, for example, in Kalos and June, Immunity (2013) 39(1):49-60, and Davis et al., Cancer J. (2015) 21(6):486-491, both of which are hereby incorporated by reference in their entirety. The skilled person is able to determine appropriate reagents and procedures for adoptive transfer of cells according to the present disclosure, for example by reference to Dai et al., J Nat Cancer Inst (2016) 108(7):djv439, which is incorporated by reference in its entirety.

The present disclosure provides methods comprising administering an immune cell comprising/expressing a CAR according to the present disclosure, or an immune cell comprising/expressing nucleic acid encoding a CAR according to the present disclosure, to a subject.

In some embodiments, the methods comprise generating an immune cell, or generating/expanding a population of immune cells. In some embodiments, the methods comprise modifying an immune cell to comprise/express a CAR according to the present disclosure. In some embodiments, the methods comprise modifying an immune cell to comprise/express nucleic acid encoding a CAR according to the present disclosure.

In some embodiments, the methods comprise administering to a subject an immune cell modified to express/comprise a CAR according to the present disclosure (or modified to express/comprise a nucleic acid encoding such a CAR).

In some embodiments, the methods comprise:
(a) modifying an immune cell to express or comprise a CAR according to the present disclosure, or to express or comprise nucleic acid encoding a CAR according to the present disclosure, and
(b) administering the immune cell modified to express or comprise a CAR according to the present disclosure, or modified to express or comprise a nucleic acid encoding a CAR according to the present disclosure, to a subject.

In some embodiments, the methods comprise:
(a) isolating or obtaining immune cells;
(b) modifying an immune cell to express or comprise a CAR according to the present disclosure, or to express or comprise nucleic acid encoding a CAR according to the present disclosure, and
(c) administering the immune cell modified to express or comprise a CAR according to the present disclosure, or modified to express or comprise a nucleic acid encoding a CAR according to the present disclosure, to a subject.

In some embodiments, the methods comprise:
(a) isolating immune cells (e.g. PBMCs) from a subject;
(b) generating/expanding a population of immune cells;
(c) modifying an immune cell to express or comprise a CAR according to the present disclosure, or to express or comprise nucleic acid encoding a CAR according to the present disclosure, and
(d) administering the immune cell modified to express or comprise a CAR according to the present disclosure, or modified to express or comprise a nucleic acid encoding a CAR according to the present disclosure, to a subject.

In some embodiments, the subject from which the immune cells (e.g. PBMCs) are isolated is the same subject to which cells are administered (i.e., adoptive transfer may be of autologous/autogeneic cells). In some embodiments, the subject from which the immune cells (e.g. PBMCs) are isolated is a different subject to the subject to which cells are administered (i.e., adoptive transfer may be of allogeneic cells).

In some embodiments the methods may comprise one or more of:
obtaining a blood sample from a subject;
isolating immune cells (e.g. PBMCs) from a blood sample which has been obtained from a subject;
generating/expanding a population of immune cells;
culturing the immune cells in in vitro or ex vivo cell culture;
modifying an immune cell to express or comprise a CAR according to the present disclosure, or to express or comprise a nucleic acid encoding a CAR according to the present disclosure (e.g. by transduction with a viral vector encoding such CAR, or a viral vector comprising such nucleic acid);
culturing immune cells expressing/comprising a CAR according to the present disclosure, or expressing/comprising a nucleic acid encoding a CAR according to the present disclosure in in vitro or ex vivo cell culture;
collecting/isolating immune cells expressing/comprising a CAR according to the present disclosure, or expressing/comprising a nucleic acid encoding a CAR according to the present disclosure;
formulating immune cells expressing/comprising a CAR according to the present disclosure, or a nucleic acid encoding a CAR according to the present disclosure to a pharmaceutical composition, e.g. by mixing the cells with a pharmaceutically acceptable adjuvant, diluent, or carrier;
administering immune cells expressing/comprising a CAR according to the present disclosure, or expressing/comprising a nucleic acid encoding a CAR according to the present disclosure, or a pharmaceutical composition comprising such cells, to a subject.

In some embodiments, the methods may additionally comprise treating the cells or subject to induce/enhance expression of CAR and/or to induce/enhance proliferation or survival of immune cells comprising/expressing the CAR.

The therapeutic and/or prophylactic methods may be effective to reduce the development/progression of a disease/condition, alleviate the symptoms of a disease/condition, or reduce the pathology of a disease/condition. The methods may be effective to prevent progression of the disease/condition, e.g. to prevent worsening of, or to slow the rate of development of, the disease/condition. In some embodiments the methods may lead to an improvement in the disease/condition, e.g. a reduction in the severity of symptoms of the disease/condition, or a reduction in some other correlate of the severity/activity of the disease/condition. In some embodiments the methods may prevent development of the disease/condition to a later stage (e.g. a chronic stage or metastasis).

It will be appreciated that the therapeutic and prophylactic utility of the CAR-expressing immune cells according to the present disclosure extends to the treatment/prevention of any disease/condition that would derive therapeutic or prophylactic benefit from a reduction in the number/activity of cells expressing/overexpressing the target antigen of the CAR, and/or the number/activity of cells infected with the virus.

In some embodiments, the disease/condition to be treated/prevented in accordance with the present disclosure in which the target antigen for the CAR is pathologically implicated. That is, in some embodiments the disease/condition is a disease/condition which is caused or exacerbated by the expression/overexpression of the target antigen, a disease/condition for which expression/overexpression of the target antigen is a risk factor and/or a disease/condition for which expression/overexpression of the target antigen is positively associated with onset, development, progression, and/or severity of the disease/condition.

In certain embodiments, cells produced by methods of the disclosure are provided to an individual in need thereof for treatment of a medical condition. As used herein “treatment” or “treating,” includes any beneficial or desirable effect on the symptoms or pathology of a disease or pathological condition, and may include even minimal reductions in one or more measurable markers of the disease or condition being treated, e.g., cancer. Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

The individual being treated may be known to have cancer, suspected of having cancer, or at risk for having cancer (such as personal or family history; being sexually active, including sexually promiscuous; and/or having a genetic predisposition, including one or more specific markers). The individual may have a benign or malignant neoplasm. The individual may have early or late stage cancer, and the skilled artisan is aware that the methods of producing the cells may be tailored for such different stages of cancer, such as by utilizing peptides for the APCs that are from antigens associated with early vs. late stage cancer. In specific embodiments, the cancer may be primary, metastatic, recurrent, refractory, and so forth.

In some embodiments, one or more administrations of the cells produced by methods of the disclosure are provided to an individual in need thereof. The length of time between different administrations may be of any suitable duration, including on the order of 1-7 days, 1-4 weeks, 1-12 months, or 1, 2, 3, 4, 5, 6, 7, 8, 9, 10, or more years. Multiple infusions within about a year may be employed, in some cases. In cases wherein more than one administration of cells are provided to the individual, the antigen to which the cells are targeted may or may not be the same antigen that was targeted with the cells utilized in earlier administration(s). Additional administration(s) may be required in cancers that become refractory, for example. Additional stimulations may be employed in conjunction with one or more other types of cancer treatments.

Methods of medical treatment may involve treatment of cancer by a method of ameliorating, treating, or preventing a malignancy in a human subject wherein the steps of the method assist or boost the immune system in eradicating cancerous cells. Such methods may include the administration of cells, according to the present invention that invoke an active (or achieve a passive) immune response to destroy cancerous cells. Methods of treatment may optionally include the co-administration of biological adjuvants (e.g., interleukins, cytokines, Bacillus Comette-Guerin, monophosphoryl lipid A, etc.) in combination with conventional therapies for treating cancer such as chemotherapy, radiation, or surgery. Methods of treatment may involve administering a composition according to the present invention as a vaccine that works by activating the immune system to prevent or destroy cancer cell growth. Methods of medical treatment may also involve in vivo, ex vivo, and adoptive immunotherapies, including those using autologous and/or heterologous cells or immortalized cell lines.

In accordance with this disclosure, the term “pharmaceutical composition” relates to a composition for administration to an individual. In a particular embodiment, the pharmaceutical composition comprises a composition comprising therapeutic immune cells for parenteral, transdermal, intraluminal, intra-arterial, intrathecal or intravenous administration or for direct injection into a neoplasm, such as a cancer. It is in particular envisaged that the pharmaceutical composition is administered to the individual via infusion or injection. Administration of the suitable compositions may be affected by different ways, e.g., by intravenous, subcutaneous, intraperitoneal, intramuscular, topical or intradermal administration.

The pharmaceutical composition of the present disclosure may further comprise a pharmaceutically acceptable carrier. Examples of suitable pharmaceutical carriers are well known in the art and include phosphate buffered saline solutions, water, emulsions, such as oil/water emulsions, various types of wetting agents, sterile solutions, etc. Compositions comprising such carriers can be formulated by well-known conventional methods. These pharmaceutical compositions can be administered to the subject at a suitable dose.

The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. A particular dosage for administration might be in the range of 2×107 cells per m2 to 1×1010 cells per m2 of body surface area. Progress can be monitored by periodic assessment.

The compositions of the disclosure may be administered locally or systemically. In a preferred embodiment, the pharmaceutical composition is administered subcutaneously and in an even more preferred embodiment intravenously. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishes, electrolyte replenishers (such as those based on Ringer's dextrose), and the like.

Preservatives and other additives may also be present such as, for example, antimicrobials, anti-oxidants, chelating agents, and inert gases and the like. In addition, the pharmaceutical composition of the present disclosure might comprise proteinaceous carriers, like, e.g., serum albumin or immunoglobulin, preferably of human origin. It is envisaged that the pharmaceutical composition of the disclosure might comprise, in addition to the cells as described in this disclosure, further biologically active agents, depending on the intended use of the pharmaceutical composition.

### Functional Definitions

The polypeptides described herein may be characterised by reference to certain functional properties. In some embodiments, the polypeptides are vitronectin, RetroNectin®, and/or a polypeptide having at least 70% sequence identity to one of SEQ ID NO:1, SEQ ID NO:2, or SEQ ID NO:3, or a polypeptide comprising an amino acid sequence having at least 70% sequence identity to a fragment of SEQ ID NO:3, SEQ ID NO:2, or SEQ ID NO:1. In some embodiments, the polypeptides described herein may possess one or more of the following properties:
Increases cell adhesion
Increases cell adhesion to a surface
Increases cell adhesion to a culture vessel
Increases cell adhesion to an inner surface of a culture vessel.
Increases cell adhesion compared to the cell adhesion of control cells cultured in the same way but without the polypeptide, as assessed by microscopy, a flow chamber pressure assay, a non-invasive and label-free real-time impedance assay, or through an assay utilising anti-cell adhesion antibodies.
Increases cell adhesion by 1.5 times compared to the cell adhesion of control cells cultured in the same way but without the polypeptide Increases cell adhesion by one of 2 times, 3 times, 4 times, or 5 times compared to the cell adhesion of control cells cultured in the same way but without the polypeptide
Increases cell adhesion in the absence of fetal bovine serum (FBS)
Increases cell adhesion in FBS-free media
Increases cell adhesion in the absence of serum
Increases cell adhesion in serum-free media
Increases cell adhesion in the absence of animal products
Increases cell adhesion in animal-product-free media
Increases cell adhesion in xeno-free media
Reduces cellular aggregation
Reduces cellular aggregation compared to the aggregation of control cells cultured in the same way but without the polypeptide, as assessed by microscopy.
Reduces cellular aggregation by 1.5 times compared to the aggregation of control cells cultured in the same way but without the polypeptide.
Reduces cellular aggregation by 2 times compared to the aggregation of control cells cultured in the same way but without the polypeptide
Increases viral titre
Increases viral titre in culture supernatant
Increases viral titre in the culture supernatant after transfection
Increases viral titre in the culture supernatant after transfection with a plasmid containing a gene of interest
Increases viral titre in the culture supernatant after 6 hours of post-transfection incubation
Increases viral titre in the culture supernatant after 12 hours of post-transfection incubation
Increases viral titre in the culture supernatant after 24 hours of post-transfection incubation
Increases viral titre in the culture supernatant after 48 hours of post-transfection incubation
Increases viral titre in the culture supernatant after 72 hours of post-transfection incubation
Increases viral titre when the polypeptide is used compared to the viral titre produced by control cells cultured in the same way but without the polypeptide.
Increases viral titre by over 1.5 times when the polypeptide is used compared to the viral titre produced by control cells cultured in the same way but without the polypeptide.
Increases viral titre by one of over 2 times, 3 times, 4 times, 5 times, 6 times, or 7 times when the polypeptide is used compared to the viral titre produced by control cells cultured in the same way but without the polypeptide.
Increases/enhances viral-mediated gene transduction
Increases/enhances viral-mediated gene transduction of mammalian cells
Increases transduction efficiency compared with transduction without the use of the polypeptide
Increases transduction efficiency compared with transduction without the use of a transduction enhancer
Improves transduction efficiency by one of at least 1.75, 2.0, 2.25, 2.5, 2.75, 3.0, 3.25, 3.5, 3.75, or 4.0 times compared with transduction without the use of a transduction enhancer
Improves transduction efficiency by one of at least 1.1, 1.2, 1.3, 1.4, 1.5, 1.6, 1.7, 1.8, 1.9, 2.0, 2.25, 2.5, 2.75, 3.0, 3.25, 3.5, 3.75, or 4.0 times compared with transduction without the use of a transduction enhancer
Improves transduction efficiency by one of at least 1.1 times compared with transduction without the use of a transduction enhancer
Improves transduction efficiency by one of at least 1.75, 2.0, 2.25, 2.5, 2.75, 3.0, 3.25, 3.5, 3.75, or 4.0 times compared with transduction without the use of the polypeptide
Improves transduction efficiency by one of at least 1.1, 1.2, 1.3, 1.4, 1.5, 1.6, 1.7, 1.8, 1.9, 2.0, 2.25, 2.5, 2.75, 3.0, 3.25, 3.5, 3.75, or 4.0 times compared with transduction without the use of the polypeptide
Improves transduction efficiency by one of at least 1.1 times compared with transduction without the use of the polypeptide
Reduces cytokine secretion from cells
Reduces cytokine secretion from effector immune cells
Reduces cytokine secretion from cells transduced with a chimeric antigen receptor (CAR)
Reduces cytokine secretion from cells used in adoptive cell transfer
Reduces cytokine secretion from CAR-T cells
Reduces cytokine secretion from cells whilst maintaining cytotoxicity
Reduces cytokine secretion from cells without reducing target cell killing
Improves the cell killing properties of effector immune cells
Improves the cell killing properties of cells transduced with a CAR
Improves the cell killing properties of cells used in adoptive cell transfer
Improves the cell killing properties of CAR T cells
Reduces cytokine secretion from effector immune cells compared to a cell that is not contacted with a polypeptide according to this disclosure
Reduces cytokine secretion from cells transduced with a chimeric antigen receptor (CAR) compared to a cell that is not contacted with a polypeptide according to this disclosure Reduces cytokine secretion from cells used in adoptive cell transfer compared to a cell that is not contacted with a polypeptide according to this disclosure
Reduces cytokine secretion from CAR-T cells compared to a cell that is not contacted with a polypeptide according to this disclosure
Reduces IL-6, TNF-α, IFN-γ, and/or IL-8 secretion from cells
Reduces IL-6, TNF-α, IFN-γ, and/or IL-8 secretion from effector immune cells
Reduces IL-6, TNF-α, IFN-γ, and/or IL-8 secretion from cells transduced with a chimeric antigen receptor (CAR)
Reduces IL-6, TNF-α, IFN-γ, and/or IL-8 secretion from cells used in adoptive cell transfer
Reduces IL-6, TNF-α, IFN-γ, and/or IL-8 secretion from CAR-T cells
Reduces IL-6, TNF-α, IFN-γ, and/or IL-8 secretion from cells whilst maintaining cytotoxicity
Reduces IL-6, TNF-α, IFN-γ, and/or IL-8 secretion from cells without reducing target cell killing
Reduces IL-6, TNF-α, IFN-γ, and/or IL-8 secretion from effector immune cells compared to a cell that is not contacted with a polypeptide according to this disclosure
Reduces IL-6, TNF-α, IFN-γ, and/or IL-8 secretion from cells transduced with a chimeric antigen receptor (CAR) compared to a cell that is not contacted with a polypeptide according to this disclosure
Reduces IL-6, TNF-α, IFN-γ, and/or IL-8 secretion from cells used in adoptive cell transfer compared to a cell that is not contacted with a polypeptide according to this disclosure
Reduces IL-6, TNF-α, IFN-γ, and/or IL-8 secretion from CAR-T cells compared to a cell that is not contacted with a polypeptide according to this disclosure
Improves the cell killing properties of effector immune cells compared to an effector immune cell that is not contacted with a polypeptide according to this disclosure
Improves the cell killing properties of cells transduced with a CAR compared to a cell that is not contacted with a polypeptide according to this disclosure
Improves the cell killing properties of cells used in adoptive cell transfer compared to a cell that is not contacted with a polypeptide according to this disclosure
Improves the cell killing properties of CAR T cells compared to a cell that is not contacted with a polypeptide according to this disclosure
Improves the target cell killing of cells transduced with a CAR compared to a cell that is not contacted with a polypeptide according to this disclosure
Improves the target cell killing of cells used in adoptive cell transfer compared to a cell that is not contacted with a polypeptide according to this disclosure
Improves the target cell killing of CAR T cells compared to a cell that is not contacted with a polypeptide according to this disclosure

The polypeptides described herein can have more than one of the above functional properties, and the above functional properties can be combined.

### Sequence Identity

As used herein, ‘sequence identity’ refers to the percent of nucleotides/amino acid residues in a subject sequence that are identical to nucleotides/amino acid residues in a reference sequence, after aligning the sequences and, if necessary, introducing gaps, to achieve the maximum percent sequence identity between the sequences. Pairwise and multiple sequence alignment for the purposes of determining percent sequence identity between two or more amino acid or nucleic acid sequences can be achieved in various ways known to a person of skill in the art, for instance, using publicly available computer software such as ClustalOmega (Sdding, J. Bioinformatics (2005) 21, 951-960), T-coffee (Notredame et al. J. Mol. Biol. (2000) 302, 205-217), Kalign (Lassmann and Sonnhammer 2005, BMC Bioinformatics, 6(298)) and MAFFT (Katoh and Standley, Molecular Biology and Evolution (2013) 30(4) 772-780) software. When using such software, the default parameters, e.g. for gap penalty and extension penalty, are preferably used.

### SEQUENCES

| SEQ ID NO | Identifier | Sequence |
|---|---|---|
| 1 | Fibronectin | PTDLRFTNIGPDTMRVTWAPPPSIDLTNFLVRYSPVKNEEDVAELSISP |
| | fragment CH-296 | SDNAVVLTNLLPGTEYVVSVSSVYEQHESTPLRGRQKTGLDSPTGID |
| | (RetroNectin ®) | FSDITANSFTVHWIAPRATITGYRIRHHPEHFSGRPREDRVPHSRNSIT |
| | amino acid | LTNLTPGTEYVVSIVALNGREESPLLIGQQSTVSDVPRDLEVVAATPTS |
| | sequence | LLISWDAPAVTVRYYRITYGETGGNSPVQEFTVPGSKSTATISGLKPG |
| | | VDYTITVYAVTGRGDSPASSKPISINYRTEIDKPSMAIPAPTDLKFTQVT |
| | | PTSLSAQWTPPNVQLTGYRVRVTPKEKTGPMKEINLAPDSSSVVVSG |
| | | LMVATKYEVSVYALKDTLTSRPAQGVVTTLENVSPPRRARVTDATET |
| | | TITISWRTKTETITGFQVDAVPANGQTPIQRTIKPDVRSYTITGLQPGTD |
| | | YKIYLYTLNDNARSSPVVIDASTAIDAPSNLRFLATTPNSLLVSWQPPR |
| | | ARITGYIIKYEKPGSPPREVVPRPRPGVTEATITGLEPGTEYTIYVIALK |
| | | NNQKSEPLIGRKKTDELPQLVTLPHPNLHGPEILDVPST |
| 2 | Human vitronectin | DQESCKGRCTEGFNVDKKCQCDELCSYYQSCCTDYTAECKPQVTR |
| | lacking signal | GDVFTMPEDEYTVYDDGEEKNNATVHEQVGGPSLTSDLQAQSKGNP |
| | peptide (P04004)- | EQTPVLKPEEEAPAPEVGASKPEGIDSRPETLHPGRPQPPAEEELCS |
| | amino acid | GKPFDAFTDLKNGSLFAFRGQYCYELDEKAVRPGYPKLIRDVWGIEG |
| | sequence (459 | PIDAAFTRINCQGKTYLFKGSQYWRFEDGVLDPDYPRNISDGFDGIPD |
| | amino acids) | NVDAALALPAHSYSGRERVYFFKGKQYWEYQFQHQPSQEECEGSSL |
| | | SAVFEHFAMMQRDSWEDIFELLFWGRTSAGTRQPQFISRDWHGVPG |
| | | QVDAAMAGRIYISGMAPRPSLAKKQRFRHRNRKGYRSQRGHSRGRN |
| | | QNSRRPSRATWLSLFSSEESNLGANNYDDYRMDWLVPATCEPIQSV |
| | | FFFSGDKYYRVNLRTRRVDTVDPPYPRSIAQYWLGCPAPGHL |
| 3 | Human Vitronectin | MAPLRPLLILALLAWVALADQESCKGRCTEGFNVDKKCQCDELCSYY |
| | (D9ZGG2)-amino | QSCCTDYTAECKPQVTRGDVFTMPEDEYTVYDDGEEKNNATVHEQV |
| | acid sequence (478 | GGPSLTSDLQAQSKGNPEQTPVLKPEEEAPAPEVGASKPEGIDSRPE |
| | amino acids) | TLHPGRPQPPAEEELCSGKPFDAFTDLKNGSLFAFRGQYCYELDEKA |
| | | VRPGYPKLIRDVWGIEGPIDAAFTRINCQGKTYLFKGSQYWRFEDGVL |
| | | DPDYPRNISDGFDGIPDNVDAALALPAHSYSGRERVYFFKGKQYWEY |
| | | QFQHQPSQEECEGSSLSAVFEHFAMMQRDSWEDIFELLFWGRTSAG |
| | | TRQPQFISRDWHGVPGQVDAAMAGRIYISGMAPRPSLAKKQRFRHR |
| | | NRKGYRSQRGHSRGRNQNSRRPSRATWLSLFSSEESNLGANNYDD |
| | | YRMDWLVPATCEPIQSVFFFSGDKYYRVNLRTRRVDTVDPPYPRSIA |
| | | QYWLGCPAPGHL |
| 4 | ITAM consensus | YXXL/I |
| | | wherein X = any amino acid |
| 5 | Larger ITAM | YXXL/I(X)6-8YXXL/I |
| | consensus | wherein X = any amino acid |
| 6 | P1 | CCTDYTAECKPQVTRGDVFTMPEDE |
| 7 | P2 | CCRRPQVTRGDVFTMPWLC |
| 8 | P2 (fragment) | PQVTRGDVFTMP |
| 9 | P6 | KKALLILALLAHWVALAKK |
| 10 | P6 (fragment) | LLILALLAWVALA |
| 11 | P6 (fragment with | LLILALLAHWVALA |
| | insertion) | |
| 12 | P7 | RPSLAKKQRFRHRNRKGYRSQRGHSRGRNQNSRR |
| 13 | PR1 | DYTITVYAVTGRGDSPASSKPIS |
| 14 | PR7.1 | PAPTDLKFTQVTPTSLSAQWTPPNVQLTGYRVRVTPKEKTGPMKEIN |
| | | LAPDSSSVVVSGLMVATKYEVSVYALKDTLTSRPAQGVVTTLENVSP |
| | | PRRARVTDATETTITISWRTKTETITGFQVDAVPANGQTPIQRTIKPDV |
| | | RSYTITGLQPGTDYKIYLYTLNDNARSSPVVIDAST |
| 15 | PR7.2 | PAPTDLKFTQVTPTSLSAQWTPPNVQLTGYRVRVTPKEKTGPMKEIN |
| | | LAPDSSSVVVSGLMVATKYEVSVYALKDTLTSRPAQGVVTTLE |
| 16 | CV-RV#1 (full | MAPLRPLLILALLAWVALADQESCKGRCTEGFNVDKKCQCDELCSYY |
| | sequence) | QSCCTDYTAECKPQVTRGDVFTMPAPTDLKFTQVTPTSLSAQWTPP |
| | | NVQLTGYRVRVTPKEKTGPMKEINLAPDSSSVVVSGLMVATKYEVSV |
| | | YALKDTLTSRPAQGVVTTLEEPKSADKTHTCPPCPAPELLGGPSVFLF |
| | | PPKPKDTLMISRTPEVTCVVVDVSHEDPEVKFNWYVDGVEVHNAKTK |
| | | PREEQYNSTYRVVSVLTVLHQDWLNGKEYKCKVSNKALPAPIEKTISK |
| | | AKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEA |
| | | LHNHYTQKSLSLSPGK |
| 17 | CV-RV#2 (full | MAPLRPLLILALLAWVALADQESCKGRCTEGFNVDKKCQCDELCSYY |
| | sequence) | QSCCTDYTAECKPQVTRGDVFTMPAPTDLKFTQVTPTSLSAQWTPP |
| | | NVQLTGYRVRVTPKEKTGPMKEINLAPDSSSVVVSGLMVATKYEVSV |
| | | YALKDTLTSRPAQGVVTTLENVSPPRRARVTDATETTITISWRTKTETI |
| | | TGFQVDAVPANGQTPIQRTIKPDVRSYTITGLQPGTDYKIYLYTLNDNA |
| | | RSSPVVIDASTEPKSADKTHTCPPCPAPELLGGPSVFLFPPKPKDTLM |
| | | ISRTPEVTCVVVDVSHEDPEVKFNWYVDGVEVHNAKTKPREEQYNST |
| | | YRVVSVLTVLHQDWLNGKEYKCKVSNKALPAPIEKTISKAKGQPREP |
| | | QVYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |
| 18 | Recombinant | MAPLRPLLILALLAWVALADQESCKGRCTEGFNVDKKCQCDELCSYY |
| | vitronectin/ | QSCCTDYTAECKPQVTRGDVFTMPAPTDLKFTQVTPTSLSAQWTPP |
| | RetroNectin | NVQLTGYRVRVTPKEKTGPMKEINLAPDSSSVVVSGLMVATKYEVSV |
| | peptide | YALKDTLTSRPAQGVVTTLENVSPPRRARVTDATETTITISWRTKTETI |
| | | TGFQVDAVPANGQTPIQRTIKPDVRSYTITGLQPGTDYKIYLYTLNDNA |
| | | RSSPVVIDAST |
| 19 | RetroNectin ® | APTDLKFTQVTPTSLSAQWTPPNVQLTGYRVRVTPKEKTGPMKEINL |
| | peptide (domains | APDSSSVVVSGLMVATKYEVSVYALKDTLTSRPAQGVVTTLENVSPP |
| | 12-14) | RRARVTDATETTITISWRTKTETITGFQVDAVPANGQTPIQRTIKPDVR |
| | | SYTITGLQPGTDYKIYLYTLNDNARSSPVVIDAST |
| 20 | RetroNectin® | APTDLKFTQVTPTSLSAQWTPPNVQLTGYRVRVTPKEKTGPMKEINL |
| | peptide (domains | APDSSSVVVSGLMVATKYEVSVYALKDTLTSRPAQGVVTTLE |
| | 12-13) | |
| 21 | Vitronectin N- | MAPLRPLLILALLAWVALADQESCKGRCTEGFNVDKKCQCDELCSYY |
| | terminal peptide | QSCCTDYTAECKPQVTRGDVFTMP |
| | with signal peptide | |
| 22 | Vitronectin signal | MAPLRPLLILALLAWVALA |
| | peptide | |
| 23 | Vitronectin N- | DQESCKGRCTEGFNVDKKCQCDELCSYYQSCCTDYTAECKPQVTR |
| | terminal peptide | GDVFTMP |
| 24 | P3 | LAQHEQGGFNNNFNTSNWSSAQVN |
| 25 | P4 (control) | KKALLAHALHLLALLALHLAHALKKA |
| 26 | P5 | PLLILALLAWVALADQESCKGRC |
| 27 | Linker | EPKSA |

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### NUMBERED STATEMENTS

1. A cell culture method, the method comprising adherent culture of cells in a culture vessel, wherein the cells are cultured in culture media that is a fetal bovine serum (FBS)-free media and the culture vessel contains a polypeptide having at least 70% sequence identity to one of SEQ ID NO:1, SEQ ID NO:2, or SEQ ID NO:3, and wherein one or more of the cells expresses a viral vector.

2. The method of statement 1, wherein the viral vector is a lentiviral vector, retroviral vector, adeno-associated viral (AAV) vector, or adenoviral vector.

3. The method of statement 1 or statement 2, wherein the cell is a mammalian or insect cell.

4. The method of any previous statement, wherein the polypeptide has at least 75% sequence identity to one of SEQ ID NO:1, SEQ ID NO:2, or SEQ ID NO:3, preferably wherein the polypeptide has one of at least 80%, 85%, 90%, 95%, or 99% sequence identity to one of SEQ ID NO:1, SEQ ID NO:2, or SEQ ID NO:3.

5. The method of any previous statement, wherein the polypeptide is present in the culture media.

6. The method of any previous statement, wherein the polypeptide is provided in the form of a liquid solution.

7. The method of any previous statement, wherein the polypeptide is coated on a surface of the culture vessel.

8. The method of any previous statement, wherein the culture vessel is a culture plate, a culture dish, a culture flask, or a bioreactor.

9. The method of any previous statement, wherein the polypeptide is provided as an additive to FBS-free media.

10. The method of any previous statement, wherein the culture media does not contain animal serum.

11. The method of any previous statement, wherein the cells have been transfected with a packaging helper plasmid.

12. The method of any previous statement, wherein the cells have been transfected with a plasmid encoding a gene of interest.

13. The method of statement 12, wherein the gene of interest is a chimeric antigen receptor (CAR) gene.

14. The method of any one of statements 3 to 13, wherein the mammalian cell is a human cell.

15. The method of statement 14, wherein the human cell is a human embryonic kidney cell, HeLa cell, or HT-1080 cell.

16. The method of statement 15, wherein the human embryonic kidney cell is a HEK293 cell, HEK293T cell, or HEK293FT cell.

17. A method of producing a viral vector, the method comprising culturing cells according to the method of any previous statement, the method further comprising the step of collecting a virus, a viral vector, and/or a viral particle from the culture.

18. A method of producing a cell for use in adoptive cell therapy comprising the following steps:
(i) producing a viral vector according to the method of statement 17, and
(ii) transducing an immune cell with the viral vector to generate a cell for use in adoptive cell therapy.

19. The method of statement 18, wherein steps (i) and (ii) are performed without changing media.

20. The method of statement 18 or statement 19, wherein FBS-free media is used in steps (i) and (ii).

21. The method of any one of statements 18 to 20, wherein step (ii) is performed in the presence of a polypeptide having at least 70% sequence identity to one of SEQ ID NO:1, SEQ ID NO:2 or SEQ ID NO:3.

22. A method of transducing an immune cell with a viral vector encoding a gene of interest, the method comprising contacting immune cells with a viral vector encoding the gene of interest and a polypeptide having at least 70% sequence identity to one of SEQ ID NO:1, SEQ ID NO:2 or SEQ ID NO:3, under conditions suitable for transduction of the cells with said viral vector to generate a transduced cell for use in adoptive cell therapy.

23. The method of statement 22, wherein the polypeptide is vitronectin.

24. The method of statement 22 or statement 23, wherein the immune cell is a mammalian immune cell.

25. The method of any one of statements 22 to 24, wherein the mammalian immune cell is a human immune cell.

26. The method of any one of statements 22 to 25, wherein the cell for use in adoptive cell therapy is a CAR expressing cell.

27. The method of statement 26, wherein the CAR expressing immune cell is a CAR T-cell or CAR-Natural Killer (CAR-NK) cell.

28. The method of any one of statements 22 to 27, further comprising the step of harvesting one or more transduced cells.

29. The method of any one of statements 22 to 28, further comprising the step of expanding the transduced cells.

30. The method of any one of statements 22 to 29, further comprising formulating transduced mammalian cells with a pharmaceutically acceptable excipient to generate a medicament.

### EXAMPLES

### Example 1—RetroNectin® Promotes Cell Adhesion for Viral Vector Production

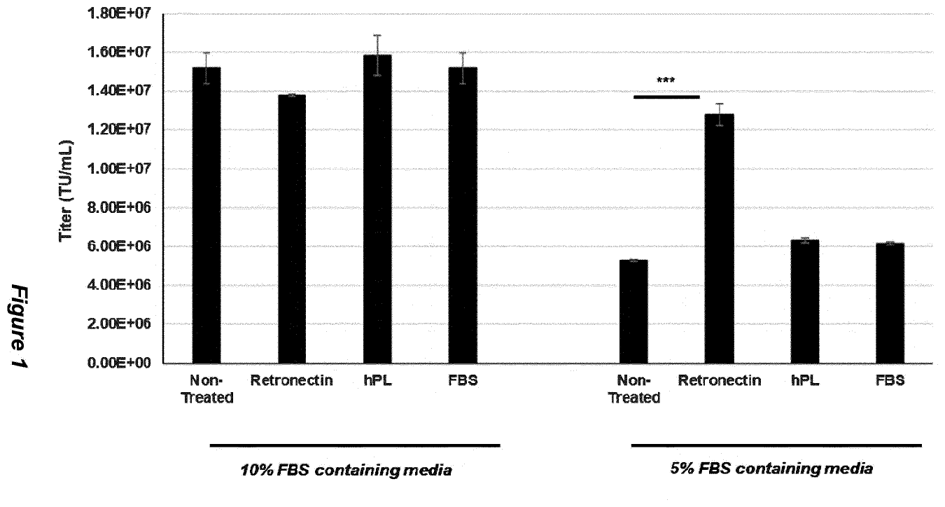

6-well plates were coated with human platelet lysates (5%; StemCell Technologies Inc.); Fetal bovine Serum (Neat; Life Technologies) and RetroNectin® (8 μg/mL; Takara Bio Inc.) overnight at 37° C. To study whether these reagents could support cell attachment in the absence of serum, both 10% and 5% (v/v) fetal bovine serum (FBS) adapted human embryonic kidney cells HEK293T (also abbreviated as 293T) were seeded in the pre-coated wells at a density of 280K per 6-well dish. Seventy-two hours post-seeding, the cells were transfected using PEI reagent and pre-optimized CellVec packaging helper plasmids (plasmids 1-3). In short, plasmid #1 carries HIV1 Rev protein, plasmid #2 carries VSV-G envelope protein, and plasmid #3 carries HIV1 gag/pol proteins. The gene of interest plasmid #4 mimics a chimeric antigen receptor with the exception that instead of a target, this was substituted by a 6×HisTag for viral vector titration purposes. Titration was performed using PE conjugated Anti-6×HisTag antibody (Abcam, 1:200) after optimization of antibody binding assay by flow cytometry. As anticipated, the results did not show significant differences in viral production when 293T cells were cultured in 10% FBS-supplemented DMEM under the various conditions. However, in reduced 5% FBS-supplemented DMEM, RetroNectin® significantly promoted 293T cell adhesion compared to all other groups (FIG. 1). In FIG. 1, the FBS group plate was coated with neat FBS over night at 37° C., the hPL group plates were coated using 5% of human platelet lysate/PBS over night at 37° C., the RetroNectin® plates were coated using 8 μg/mL of RetroNectin®/PBS over night at 4° C., and the control plates only used PBS. The results indicate that RetroNectin® is capable of substituting the factors in serum that are required to either facilitate cell adhesion and/or LV production.

The lentiviral vector supernatant was diluted in different volumes (0, 0.1 μl, 0.2 μl, 0.4 μl, 1 μl, 2 μl, 4 μl, 8 μl etc). and added to 293T cells. After 72 h the positive cell numbers expressing the surface target was detected by flow cytometry. Titer was calculated as the total number of positive cells (i.e. percentage of positive cells multiply by total cell number) divided by the volume of viral vector supernatant used, multiplied by 1000 (TU/mL).

We further examined the capabilities of RetroNectin® to facilitate 293T cell adhesion in several commercially available serum free culture media as represented below: ImmunoCult™-XF T Cell Expansion Medium (StemCells Technologies Inc.,) 4Cell Nutri-T Medium (abbreviated as NutriT; Sartorius AG), and TexMacs media (Miltenyi Biotech GmbH). Cells were grown in an incubator at 37° C. and 5% CO₂.

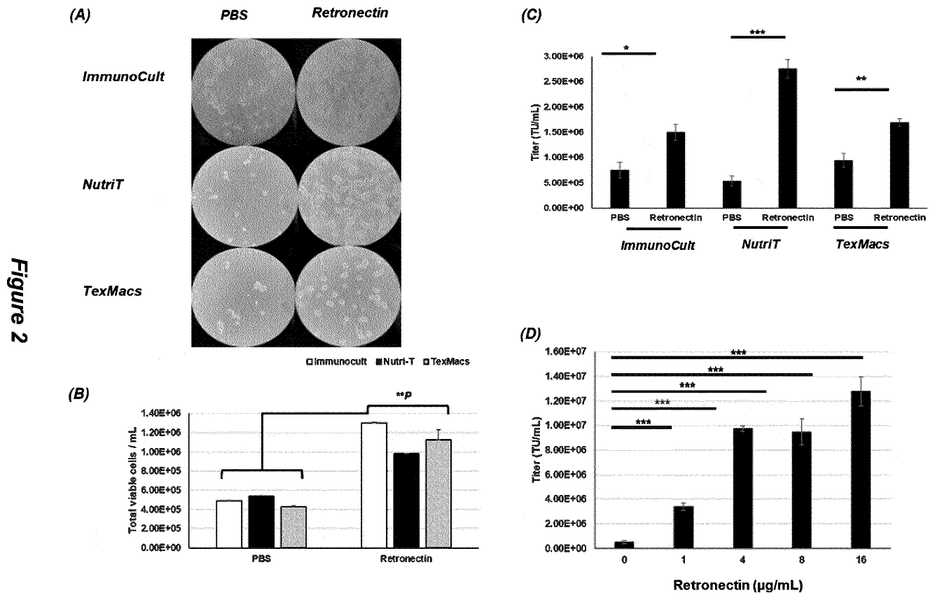

In the absence of serum, 293T rounded up and formed aggregates (FIG. 2A). Briefly, HEK293T cells were cultured in serum free conditions and were observed over a period of time to evaluate culture characteristics, using Nikon™ EclispeTS100/Leica™ DMIL or equivalent microscope. However, in the presence of pre-coating with 8 μg/mL of RetroNectin® in PBS versus no coating i.e. PBS alone, the cells spread out uniformly as a monolayer. Additionally, a significant increase in 293T cell proliferation was shown in RetroNectin® (8 μg/mL in PBS) coated 6-well plates over a period of 3 days in different types of serum free media as compared to the corresponding wells of similar culture media without any coating as controls (i.e., same volume of PBS). LV production also improved significantly in all tested serum free media in RetroNectin®-coated tissue cultureware as opposed to control PBS (FIG. 2C). NutriT could be used as a common serum free media that supports LV production and T cell expansion because it has been independently demonstrated that not only does NutriT support robust T cell expansion but the expansion skewed towards CD8+ cell populations which led to improved CAR-T potencies in vitro and in vivo.

RetroNectin® was diluted to a range of concentrations in PBS (0, 1, 4, 8 and 16 μg/mL of RetroNectin®/PBS; FIG. 2D). Coating was performed in duplicates for each of the concentrations. A total volume of 0.8 mL was used per well, and the culture dish was sealed with parafilm and placed overnight at 4° C. Studies have also shown that leaving the coating at room temperature, or 2-3 hours in the incubator also promotes cell adherence after applying the RetroNectin®. On the following day, the RetroNectin®/PBS solution was removed and 293T were seeded in serum-free NutriT media for subsequent transfection with CellVec packaging helper plasmids and plasmids #4 as mentioned above. Crude viral supernatant was harvested at 48 h post-transfection, and 2 μl and 4 μl of crude viral supernatant from each of the duplicates (from the various conditions 0, 1, 4, 8 and 16 μg/mL of RetroNectin®/PBS) was used to infect 293T cells for subsequent titration by flow cytometry analysis. The results showed that the effect of RetroNectin® in promoting viral production in the absence of serum was dose-dependent.

We next investigated whether adherent LV production is scalable from a 6-well plate (~9.6 mL volume containing ~1.5 mL media; FIG. 3A) to T75 flask (~75 mL volume containing ~10 mL media; FIG. 3B) to T175 flask (~175 mL volume containing ~25 mL media; FIG. 3C). Cells were grown in an incubator at 37° C. and 5% CO₂.

The results showed 293T cells seeded in RetroNectin®-coated culture vessels exhibited a 4-7-fold increase in LV production when compared to those without RetroNectin®. In contrast, the effect of RetroNectin® was marginal/not evident in the presence of 10% FBS-supplemented DMEM (FIG. 3A-3C). Thus, crude LV production can be produced on adherent platforms in serum free media in different culture vessels.

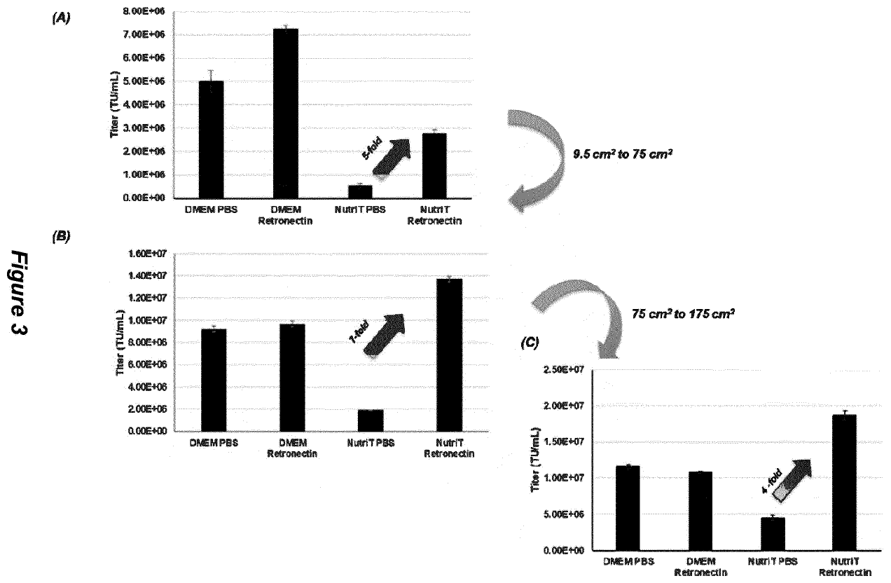

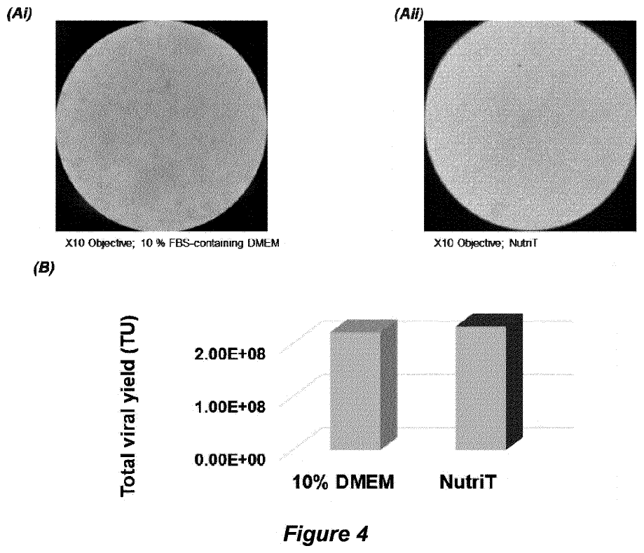

The selection of serum free media to be used for Mustang Q ion exchange chromatography membranes is pH-dependent. Kutner and Reiser have reported that there is increased virus binding to the Mustang Q ion exchange chromatography at a pH of 8.0 when compared to pH 7.0 or pH 6.0 (Molecular Therapy VOLUME 15, SUPPLEMENT 1, S313, MAY 01, 2007). We have also independently uncovered that TexMacs' pH buffering range could not be adjusted to pH8.0. As a consequence of inefficient LV binding to Mustang Q ion exchange chromatography, the recovery of the viral vector was poor. In view of this, 293T cells were seeded in NutriT serum-free media in 2-layered cell factories pre-coated with RetroNectin® at 8 μg/mL. 72 h post-seeding, the cells were transfected with CellVec packaging helper plasmids with in-house designed humanized CD19-CAR (CV-hCD19). Crude LV supernatant was clarified via a 0.45 μm filter, and purified via Mustang Q ion exchange chromatography, ultrafiltration & diafiltration steps (UF/DF), and subsequently reformulate in NutriT media. Similar experiments were performed whereby 293T cells were seeded in DMEM supplemented with 10% FBS, and underwent similar purification processes with the exception that the final reformation was performed in TexMacs media. The results showed that 293T seeded in serum-free NutriT media maintains the morphology and growth characteristics of parental HEK293T (FIG. 4Aii) and produces equivalent amounts of lentivirus compared to conventional, adherent, serum-dependent method. The total viral yield prior to centrifugation was 2.22×10\hat{ }8 and 2.33×10\hat{ }8 TU using 10% FBS-containing DMEM and serum free Nutri T media respectively (FIG. 4B).

### Example 2—Vitronectin Promotes Cell Adhesion for Viral Vector Production

Although we have clearly demonstrated the feasibility of producing viral vectors in serum free media by repurposing RetroNectin® from its usual function as transduction enhancer, pre-coating the cell factories with RetroNectin® is nonetheless an additional step that would involve more effort. A study by Matsushita and colleagues studied the effect of shear stress on the adhesion and productivity of parental 293 cells media containing FBS (Matsushita T et al., 2000. Cytotechnology 32(3):181-190). Among the various additives tested, vitronectin was found to be most robust at increasing the productivity of 293 cells exposed to shear stress when grown in media containing FBS. We therefore investigated whether vitronectin could be added as a liquid solution to facilitate the adhesion of 293T cells in the absence of FBS. Recombinant vitronectin ACF from Sartorius was used for this study. This is a 459 amino acid, single-chain, monomeric protein that can function as a chemically-defined matrix component for the attachment of human embryonic and induced pluripotent stem cells in a feeder-free culture system. Similar experiments as in FIG. 2 with RetroNectin® were performed. Vitronectin solution was added to 293T during seeding in the presence of serum-free NutriT media at 10 μg/ml. The results showed that vitronectin exhibited a similar effect as RetroNectin® in aiding the cells to spread out (FIG. 5A). Without vitronectin, 293T cells rounded up and formed clusters of cells. Additionally, a significant increase in 293T cell proliferation was seen in vitronectin (5 μg/mL in PBS) coated 6-well plates in different types of serum free media as compared to the corresponding wells of similar culture media without any coating as controls (i.e., same volume of PBS). LV production also improved significantly in all tested serum free media (FIG. 5C). Soluble vitronectin was added to a range of concentrations (0.5 μg/ml; 1 μg/mL; 5 μg/mL; 10 μg/mL and 20 μg/mL) during seeding of 293T in NutriT. Transfection was performed as FIG. 2. The results showed that LV productivity increases in a vitronectin dose-dependent (FIG. 5D). Taken together, we have demonstrated that soluble vitronectin can also be used in promoting the adherence of 293T cells in serum free conditions.

### Example 3—Vitronectin Enhances Viral-Mediated Gene Transduction

We next examined the effect of NutriT media against ImmunoCult which has been employed as a standard T cell culture media, both supplemented with 100 units/mL of IL-2, on T-cells proliferation, T cell subsets before, and post transduction with our test bed construct CV-hCD19 LV. The results showed that NutriT supported robust T cell expansion when measured at day 7 using trypan blue analysis (FIG. 6). Further Nutri T preferentially expanded CD8 cell populations under normal cell culture conditions (FIG. 6) as well as, when transduced with CV-hCD19 LV vectors. The various cell populations were stained with the respective antibodies for FACS analysis. CAR surface expression was detected by human CD19 recombinant proteins labelled with Atto 488 (R n D Systems).

Transduction efficiency in various conditions was evaluated by a flow cytometry assay. Briefly, cell populations were stained with the respective antibodies for FACS analysis. CAR surface expression was detected by human CD19 recombinant proteins labelled with Atto 488 (R n D Systems). Relative variance of CAR expression among T cells cultured in different conditions (as mentioned above) was calculated based on FACS analysis by BD FACS Suite™ or FlowJo™ (BD Biosciences).

As RetroNectin® is used as a transduction enhancer, it was next investigated whether soluble vitronectin may have a similar effect. To study this, vitronectin was coated on untreated 24-well dish as in RetroNectin® for PBMC transduction with LV using experimental conditions as indicated in the earlier sections. The results showed that vitronectin could also act as a transduction enhancer, and increase transduction efficiency of PBMCs (FIG. 6).

### Example 4—Potency of Viral Vectors

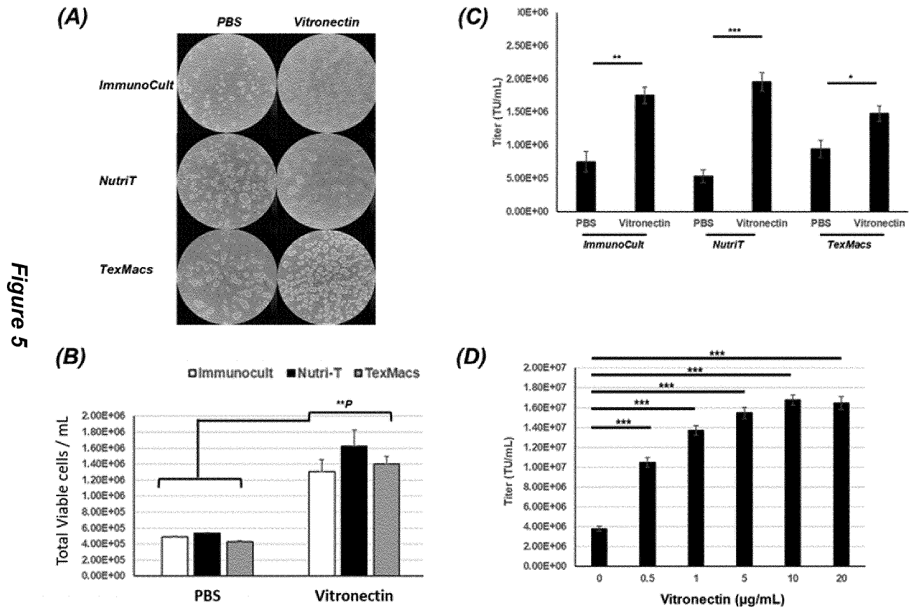

We then examined the potency of lentiviral vectors encoding humanized CD19-CAR (CV-hCD19) CAR LV produced by the conventional adherent process with 293T cultured in DMEM supplemented with 10% (v/v) FBS versus NutriT. Human peripheral blood mononuclear cells (PBMC) were activated by CD3/CD28 (72h) and transduced with CV-hCD19 at a multiplicity of infection (MOI) of 5 or left untransduced (NTC) in various conditions as labelled with either RetroNectin® or vitronectin coating of the surface of 24-well plates. CD19 expressing CAR-T-lymphocytes were then co-cultured with (FIG. 7A) Raji Cells (Burkitt Lymphoma) (FIG. 7B) Nalm6 cells (Acute Lymphoblastic Leukemia) at various Effector to Target (E:T) ratios as labelled, to evaluate cytotoxic potential.

Briefly, Nalm6-Luc or Raji-Luc cells (NALM-6 or Raji cells expressing Luciferase) were incubated with CAR-T-cells for a fixed time point. The luciferase activity of Raji-Luc or Nalm6-Luc, a measure of tumour cell viability, was recorded using BrightGlo™ Luciferase assay system (Promega, Madison, USA), according to manufacturer's instructions. The potency of CAR-T cells (manufactured and cultured in conditions described above) as a function of cytotoxicity, was evaluated based on tumour cell viability and luciferase activity after period of co-culture with Raji-Luc or Nalm6-Luc tumour cells.

The extent of cytolytic activity of CAR T-cells is presented as percentage of specific cell lysis. The results showed that CV-hCD19 LV prepared by the novel serum free adherent process (NSAP) is as efficient in killing either Raji or Nalm6 as the standard method in 10% FBS-containing DMEM. The levels of interferon-gamma secretion in the Raji cell model (FIG. 7C) and the Nalm cell model (FIG. 7D) were also comparable to the conventional method of LV production in 10% FBS. Taken together, our data demonstrate that LVs produced by NSAP is as potent as LVs produced by the standard conventional method.

### Example 5—Long Term Coating

The inventors unexpectedly found that longer coating incubations led to even greater improvements.

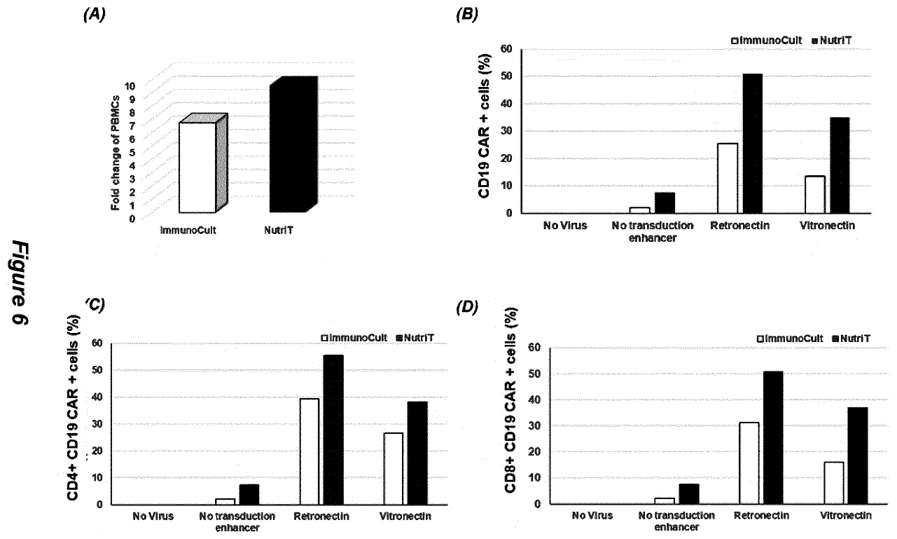

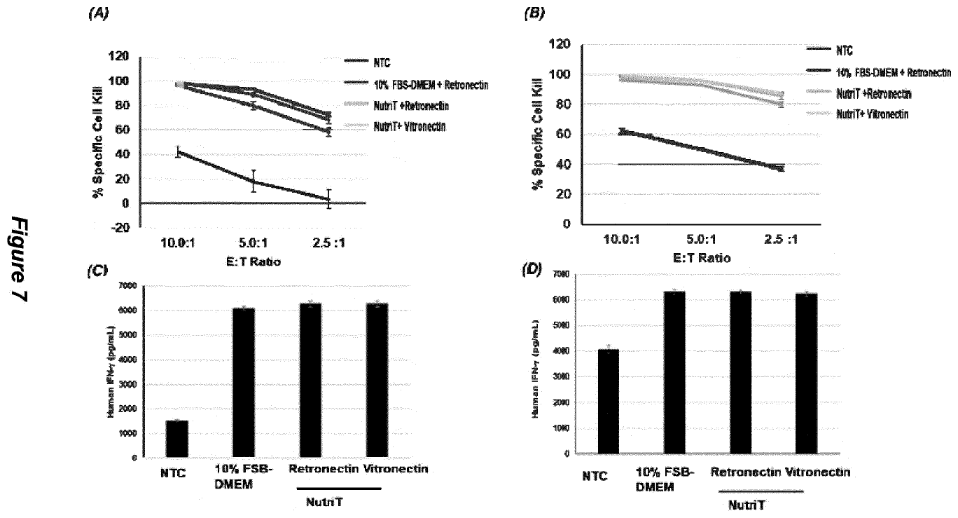

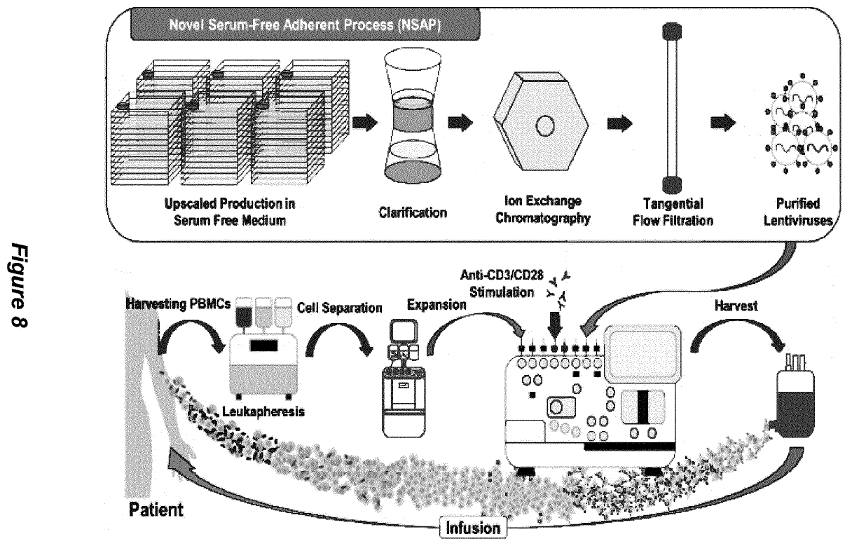

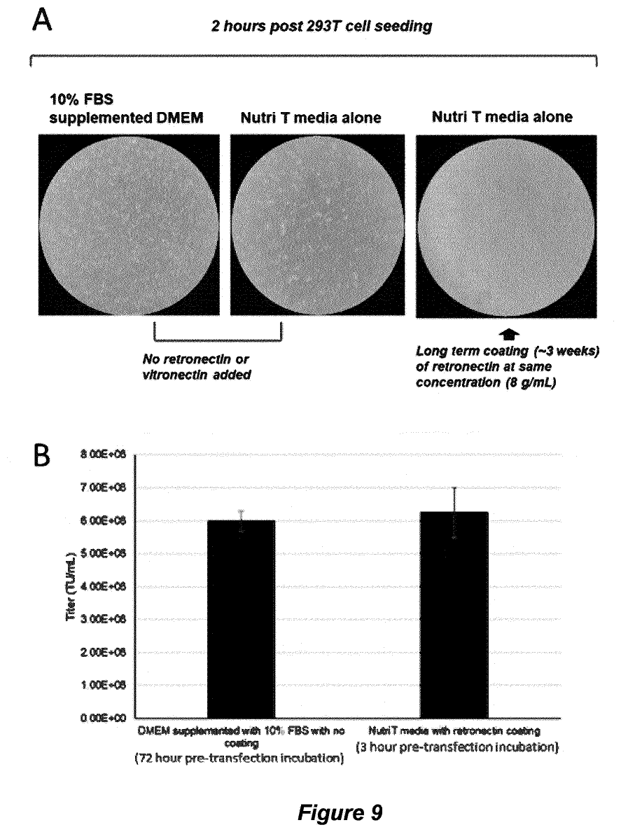

A 24-well plate coated with RetroNectin® (8 μg/mL) over a period of ~3 weeks was compared with a non-coated standard 24-well plate to assess the cell adhesion properties of 293T cells resuspended in serum free NutriT media. Cells were seeded (1×10⁵ cells) and inventors were surprised that within 3 hours, 293T cells had formed a monolayer in the RetroNectin® coated plate (FIG. 9A). This effect was not observed in plates that were not coated with RetroNectin® (DMEM supplemented with 10% FBS or Nutri T alone—FIG. 9A).

The results were shown to be reproducible. It was also found that the monolayer which formed within 3 hours could support virus productivity. The standard method for virus production is to seed 293T cells in DMEM supplemented with 10% FBS and incubate for 72 hours before transfection. This 72-hour period enables adhesion to occur. In order to ensure a comparable dataset, 293T cells were seeded using: (1) the standard 72-hour method in DMEM supplemented with 10% FBS, and (2) 3 hours before transfection in wells with long term RetroNectin® coating (after adjusting to similar cell number as the DMEM group).

The results show that virus production was as efficient when cells were seeded just 3 hours in wells with long term RetroNectin® coating as cells that were seeded 72 h before transfection. This finding has the potential to greatly reduce the overall LV production time.

### Example 6—Summary and Conclusion

Inventors have developed a novel serum-free adherent process (NSAP) for LV production (as illustrated in FIG. 8). To our knowledge, there is no published report of the production of adherent LVs using serum free conditions. The absence of bovine serum in the manufacturing process reduces the risks associated with bovine related disease transmission, and is therefore more suitable for clinical manufacturing of cell therapy products. The lower protein load, as compared to conventional bovine serum, exerts less stress on the efficiency of the downstream LV purification process due to a shorter processing time which not only leads to a safer product but also higher quality. The method also provides an alternative to other challenging factors revolving around the use of bovine serum, such as global shortages, high costs, and batch-to-batch lot variation.

### Example 7—Generation of Polypeptides Derived from Vitronectin and RetroNectin®

### 7.1 Design of Recombinant Polypeptides

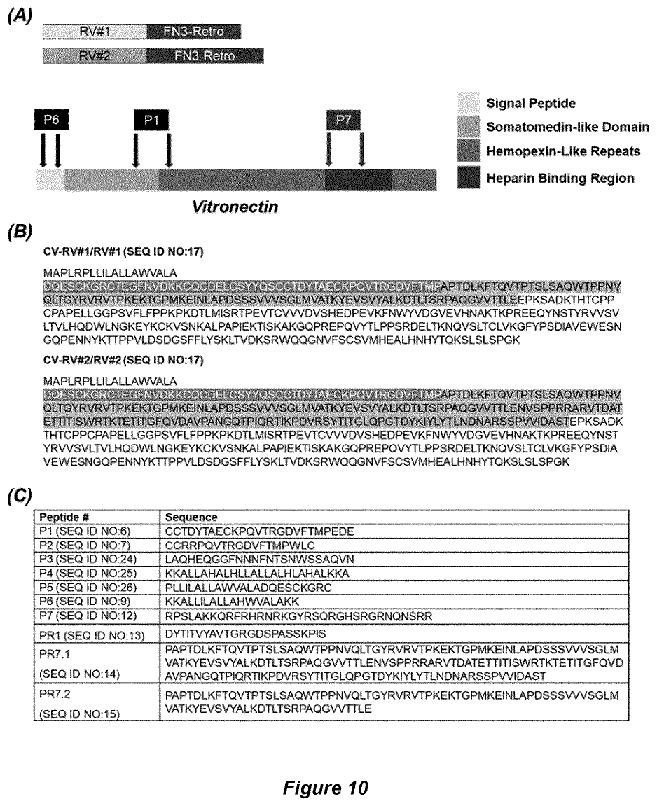

Inventors speculated that a region of RetroNectin® within the heparin-binding domain could act as the viral-binding portion, although the sequences responsible were not known. Two recombinant proteins were designed, RV #1 (SEQ ID NO:16) and RV #2 (SEQ ID NO:17), containing different lengths of the predicted viral-binding sequence (as shown in FIG. 10B). RV #1 contains RetroNectin® sequences with homology to fibronectin type III domains 12 and 13 (SEQ ID NO:20); RV #2 contains sequences corresponding to fibronectin type III domains 12-14 (SEQ ID NO:19).

A comparison between the sequences of RetroNectin® and vitronectin showed similarity at functional motifs around the “RGD” sequence. Therefore, a region of vitronectin comprising the RGD sequence, made up of the signal peptide (SEQ ID NO:22) and the first 51 amino acids at the N-terminus of vitronectin (SEQ ID NO:23), was included in the designed recombinant proteins. This sequence was incorporated at the N-terminus of RV #1 and RV #2.

These sequences were fused with human IgG via the EPKSA linker sequence (SEQ ID NO:27). Fusion of an IgG with the recombinant protein was performed in an attempt to further enhance stability and half-life, and to potentially reduce immunogenicity.

7.2 Design of Polypeptides Derived from Vitronectin

A series of peptides were also designed based on the predicted functional domains of vitronectin (SEQ ID NO:2 and SEQ ID NO:3). These peptides are shown in FIG. 1C as P1, P2, P6 and P7.

P1 (SEQ ID NO:6) and P7 (SEQ ID NO:12) are fragments of vitronectin, and both P2 (SEQ ID NO:7) and P6 (SEQ ID NO:9) are derived from fragments of vitronectin.

The P1 (SEQ ID NO:6) peptide corresponds to the sequence from vitronectin containing the RGD motif, and encompasses the boundary between the somatomedin-like domain and the central hemopexin-like domain.

The P2 (SEQ ID NO:7) peptide is based on vitronectin amino acid residues 40-52, also containing the RGD domain. This sequence is flanked at the N-terminus with CCRR and at the C-terminus with WLC.

The P6 (SEQ ID NO:9) peptide is based on the vitronectin signal peptide sequence, further modified to improve certain characteristics. Residues ‘KKA’ were added at the N-terminus of this sequence to increase the solubility of the peptide during manufacturing. An ‘H’ residue insertion was also added within the sequence, as in silico analysis found that addition of an ‘H’ residue increases the predicted bioactivity from 0.81 to 0.82.

The P7 (SEQ ID NO:12) peptide corresponds to a sequence of vitronectin falling within the C-terminal heparin binding domain.

The P4 (SEQ ID NO:25) polypeptide was provided as a control. P4, also known as LAH4 peptide or Vectofusin-1, is known to enhance viral infection, and has been used to promote Hcd34 cell-based gene therapy (Fenard, D., et al. Mol Ther Nucleic Acids. (2013) 2(5):e90).

Additional peptides, P3 (SEQ ID NO:24) and P5 (SEQ ID NO:26) were also investigated but found to be non-functional.

7.3 Design of Polypeptides Derived from RetroNectin®

Sequence alignments of the N-terminal regions of RetroNectin® and vitronectin identified conserved motifs within the cell binding domains of RetroNectin®.

Sequences of RetroNectin® were subsequently identified that correspond to the polypeptides derived from vitronectin, in particular peptides P1 and P7. These RetroNectin®-based peptides (PR1 (SEQ ID NO:13), PR7.1 (SEQ ID NO:14), and PR7.2 (SEQ ID NO:15), shown in FIG. 10C) are predicted to be transduction enhancers either in adherent or soluble method of transduction.

### Example 8—Transduction Enhancement Properties of Polypeptides Derived from Vitronectin and RetroNectin®

Commercial Human PBMCs (STEMCELL Technologies, Canada) were thawed and activated using CD3/CD28 cocktail (Miltenyi, Germany) for 72 hours.

Activated PBMCs were transduced in 24-well plates with hCD19-CAR at a multiplicity of infection (MOI) of 5 or left un-transduced (NTC), in the presence of either RetroNectin® (the standard transduction enhancer control), vitronectin, RV #1, RV #2, peptides P1, P2, P4, P6 or P7, or with no added reagents.

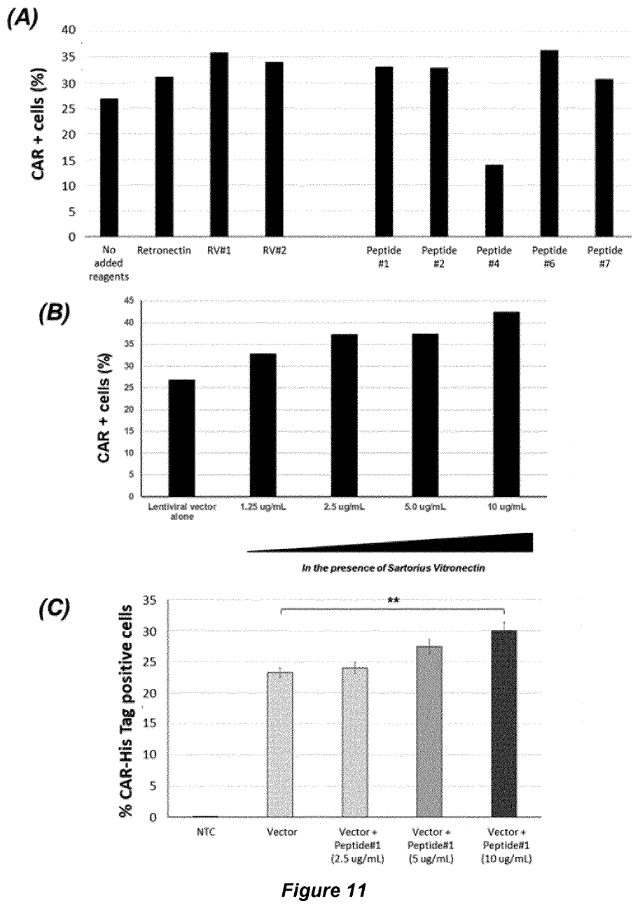

72 hours post-transduction, cultures were analysed for the percentage of CD19 CAR+ cells. Results are shown in FIG. 11A.

Cells transduced in the presence of the recombinant proteins RV #1 or RV #2 showed higher transduction efficiency compared to cells transduced with no added reagents. Cells transduced in the presence of RV #1 and RV #2 also showed greater improvement in transduction efficiency compared to cells transduced in the presence of RetroNectin®.

Cells transduced in the presence of peptides P1, P2, P6 and P7 demonstrated improved transduction efficiency compared to cells transduced with no added reagents. Interestingly, cells transduced in the presence of peptide P4—a peptide known to enhance viral infection—did not show improved transduction efficiency compared to cells transduced with no added reagents.

The improved CAR-T expression of cells transduced in the presence of various transduction enhancers was shown to occur in a dose dependent manner, as demonstrated with vitronectin and peptide P1. Results are shown in FIGS. 11B and 11C respectively.

### Example 9—Impact of Transduction Enhancers on Cytokine Secretion Following Transduction

IL-6 is known as a central mediator of Cytokine Release Syndrome (CRS) toxicity associated with CAR-T cell therapy (Tanaka et al. Immunotherapy (2016) 8(8):959-70). A challenge in the development of CAR-T cell therapies is the need to reduce the impact of cytokine release syndrome without reducing the therapeutic efficacy of the CAR-T cell treatment.

To assess how the use of vitronectin may impact CRS, IL-6 secretion from vitronectin-enhanced hCD19-CAR-transduced cultures was assessed.

Commercial Human PBMCs (STEMCELL Technologies, Canada) were thawed and activated using CD3/CD28 cocktail (Miltenyi, Germany) with IL-7 and IL-15 (Miltenyi, Germany) supplementation for 24 hours at 37° C. with 5% CO2 in an incubator, cultured using Nutri-T cell medium (Sartorius, Singapore).

Activated PBMCs were transduced either with hCD19-CAR or hCD19Del-CAR (non-functional control without signalling endodomain) in a spinfection protocol in the presence of either RetroNectin® (TakaraBio, Japan), Vitronectin (Sartorius, Singapore) or with no added reagents (control).

To measure the IL-6 secretion levels, CAR expressing T-cells from each of the conditions were co-cultured with NALM-6-Luc tumor cells expressing Luciferase at either 4- or 14-days post-transduction. After 24 hours of co-culture, cell culture supernatants were collected. An automated ELISA (ELLA Immunoassay system, ProteinSimple, USA) was performed on cell culture supernatant samples to measure IL-6 levels between transduction enhancer treated or control groups. Statistical analysis was performed using two-tailed paired Student's t-tests. P<0.05 was considered significant; P<0.01 (**); P<0.001 (***). Results are shown in FIGS. 12A and 12B.

The results showed that IL-6 secretion was significantly reduced in CAR-T cells transduced in the presence of vitronectin compared CAR-T cells transduced in the presence of RetroNectin® at Day 4 (FIG. 12A).

There was no significant difference between CAR-T cells transduced in the presence of RetroNectin® and CAR-T cells transduced without the presence of a transduction enhancer.

IL-6 secretion was low in PBMCs alone and cells transduced with the non-functional hCD19Del-CAR.

Results remained consistent at day 14 (FIG. 12B), with vitronectin-treated CAR-T cells showing significantly reduced (P<0.01) IL-6 secretion compared to RetroNectin®-treated CAR-T cells.

To confirm that this effect is due to the presence of vitronectin, IL-6 secretion was analysed in hCD19-CAR-transduced T cells, transduced in the presence of increasing amounts of vitronectin. Cells were co-cultured with NALM-6-Luc tumor cells as previously described and supernatant analysed by ELISA.

Results are shown in FIG. 12C.

Results showed that reduction in IL-6 secretion correlates with increasing concentration of vitronectin.

To assess if the reduction in IL-6 secretion would impact the potential therapeutic efficacy of the CAR-T cells, the cell killing ability of hCD19-CAR-T cells transduced in the presence of vitronectin was assessed.

72 hours post-transduction, CAR expressing T-cells from each of the conditions were co-cultured with NALM-6-Luc tumor cells expressing Luciferase. Tumour cell kill was measured based on Luciferase activity detected by Bright-Glo™ Luciferase Assay System (Promega, USA). The specific cell kill percentage was calculated from the ratio of total cytolysis to spontaneous cytolysis in each condition. Results are shown in FIG. 12D.

Importantly, the reduction in IL-6 secretion did not diminish the cell killing functions of hCD19-CAR-transduced T cells. In fact, the results showed that the percentage of specific cell kill mediated by vitronectin-enhanced hCD19-CAR-transduced T cells was significantly increased (P<0.01) when compared to Retronectin-enhanced hCD19-CAR-transduced T cells or hCD19-CAR-transduced T cells transduced without added reagents (FIG. 12D).

Similar results were observed with another NKG2D CAR construct that was used as an internal control, demonstrating that this result is independent of the CAR construct used.

The presence of vitronectin after transduction was assessed. Briefly, Human Vitronectin EIA kit (TakaraBio, USA), was employed in determination of residual vitronectin associated with Vitronectin treated samples (treated at 10 μg/mL) used in the experiments shown in FIG. 12. Samples without Vitronectin served as negative controls (Shown in the figure), whereas media blank and media spiked vitronectin served as assay controls (not shown). Trace Vitronectin could be detected (~20 ng/mL) in Day 4 harvested CAR T cells, but none detected at Day 14 harvest (FIG. 12A).

These results demonstrate that vitronectin could not only enhance CAR expression, but also minimise the use of viral vector dosage, and inhibit the release of cytokines from CAR-T cells and peripheral blood mononuclear cells, while maintaining CAR-T functionality.

To confirm that other cytokines were modulated by vitronectin in a similar way, inventors assessed the levels of three more cytokines: Tumor necrosis factor alpha (TNF-α), Interferon gamma (IFN-γ), and interleukin 8 (IL-8). Experiments were performed in the same way as the abovementioned IL-6 analysis. Results show that vitronectin-treated CAR-T cells demonstrated a considerable reduction in TNF-α, IFN-γ, and IL-8 secretion compared to untreated CAR-T cells and RetroNectin®-treated CAR-T cells (FIGS. 12F to 12H).

Inventors also assessed the level of granzymes in different treatment groups (FIG. 12I). Granzymes are beneficial to the function of CAR T cells. The level of granzymes measured by multiplex ELISA was found to be similar in both vitronectin-enhanced hCD19-CAR-transduced T cells and Retronectin-enhanced hCD19-CAR-transduced T cells, indicating that while cytokines are reduced, granzymes remained active in the vitronectin treated group.

### REFERENCES

A number of publications are cited above in order to more fully describe and disclose the invention and the state of the art to which the invention pertains. Full citations for references are provided below. The entirety of each of these references is incorporated herein.
Gandara C, Affleck V, and Stoll E A. Manufacture of Third-Generation Lentivirus for Preclinical Use, with Process Development Considerations for Translation to Good Manufacturing Practice 2018; 2018 February; 29(1):1-15. doi: 10.1089/hgtb.2017.098. Epub 2018 Jan. 24. PMID: 29212357
Matshushita T, Kawakubo Y, Sawano M, and Funatsu K, 2000. Vitronectin enhances adhesion force and t-PA production of weakly adherent 293 cells exposed to a shear stress. Cytotechnology 32(3):181-190.
Merten O W, Schweizer M, Chahal P, Kamen A A. Manufacturing of viral vectors for gene therapy: part I. Upstream processing. Pharm. Bioprocess (2014) 2:183-203
Zhou A, Huntington J A, Pannu N S, Carrell R W, and Read R J. 2003. How vitronectin binds PAI-1 to modulate fibrinolysis and cell migration. Nature Structural Biology. 10 (7): 541-4. doi:10.1038/nsb943

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press.

1. A cell culture method, the method comprising adherent culture of cells in a culture vessel, wherein the cells are cultured in culture media that is a fetal bovine serum (FBS)-free media and the culture vessel contains a polypeptide comprising an amino acid sequence having at least 70% sequence identity to one of SEQ ID NO:3, SEQ ID NO:2, or SEQ ID NO:1, or a polypeptide comprising an amino acid sequence having at least 70% sequence identity to a fragment of SEQ ID NO:3, SEQ ID NO:2, or SEQ ID NO:1.

2. The method of claim 1, wherein the polypeptide comprising an amino acid sequence having at least 70% sequence identity to a fragment of SEQ ID NO:3, SEQ ID NO:2, or SEQ ID NO:1 comprises at least 70% sequence identity to one or more of SEQ ID NOs:6-23.

3. The method of claim 1 or claim 2, wherein one or more of the cells expresses a viral vector.

4. The method of claim 3, wherein the viral vector is a lentiviral vector, retroviral vector, adeno-associated viral (AAV) vector, or adenoviral vector.

5. The method of any previous claim, wherein the cell is a mammalian or insect cell.

6. The method of any previous claim, wherein the polypeptide has at least 75% sequence identity to one of SEQ ID NO:3, SEQ ID NO:2, SEQ ID NO:1, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, and/or SEQ ID NO:23, preferably wherein the polypeptide has one of at least 80%, 85%, 90%, 95%, or 99% sequence identity to one of SEQ ID NO:3, SEQ ID NO:2, SEQ ID NO:1, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, and/or SEQ ID NO:23.

7. The method of any previous claim, wherein the polypeptide is present in the culture media.

8. The method of any previous claim, wherein the polypeptide is provided in the form of a liquid solution.

9. The method of any previous claim, wherein the polypeptide is coated on a surface of the culture vessel.

10. The method of any previous claim, wherein the culture vessel is a culture plate, a culture dish, a culture flask, or a bioreactor.

11. The method of any previous claim, wherein the polypeptide is provided as an additive to FBS-free media.

12. The method of any previous claim, wherein the culture media does not contain animal serum.

13. The method of any previous claim, wherein the cells have been transfected with a packaging helper plasmid.

14. The method of any previous claim, wherein the cells have been transfected with a plasmid encoding a gene of interest.

15. The method of claim 14, wherein the gene of interest is a chimeric antigen receptor (CAR) gene.

16. The method of any one of claims 5 to 15, wherein the mammalian cell is a human cell.

17. The method of claim 16, wherein the human cell is a human embryonic kidney cell, HeLa cell, or HT-1080 cell.

18. The method of claim 17, wherein the human embryonic kidney cell is a HEK293 cell, HEK293T cell, or HEK293FT cell.

19. A method of producing a viral vector, the method comprising culturing cells according to the method of any previous claim, the method further comprising the step of collecting a virus, a viral vector, and/or a viral particle from the culture.

20. A method of producing a cell for use in adoptive cell therapy comprising the following steps:
(i) producing a viral vector according to the method of claim 19, and
(ii) transducing an immune cell with the viral vector to generate a cell for use in adoptive cell therapy.

21. The method of claim 20, wherein steps (i) and (ii) are performed without changing media.

22. The method of claim 20 or claim 21, wherein FBS-free media is used in steps (i) and (ii).

23. The method of any one of claims 20 to 22, wherein step (ii) is performed in the presence of a polypeptide having at least 70% sequence identity to one of SEQ ID NO:3, SEQ ID NO:2 or SEQ ID NO:1, or a polypeptide comprising an amino acid sequence having at least 70% sequence identity to a fragment of SEQ ID NO:3, SEQ ID NO:2, or SEQ ID NO:1.

24. A method of producing a cell for adoptive cell therapy, the method comprising contacting a cell with a polypeptide comprising an amino acid sequence having at least 70% sequence identity to one of SEQ ID NO:3, SEQ ID NO:2 or SEQ ID NO:1, or a polypeptide comprising an amino acid sequence having at least 70% sequence identity to a fragment of SEQ ID NO:3, SEQ ID NO:2, or SEQ ID NO:1, and a viral vector encoding a gene of interest.

25. A method of transducing an immune cell with a viral vector encoding a gene of interest, the method comprising contacting immune cells with a viral vector encoding the gene of interest, and a polypeptide having at least 70% sequence identity to one of SEQ ID NO:3, SEQ ID NO:2 or SEQ ID NO:1, under conditions suitable for transduction of the cells with said viral vector to generate a transduced cell for adoptive cell therapy.

26. The method of any one of claims 23 to 25, wherein the polypeptide has at least 75% sequence identity to one of SEQ ID NO:3, SEQ ID NO:2, SEQ ID NO:1, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, and/or SEQ ID NO:23, preferably wherein the polypeptide has one of at least 80%, 85%, 90%, 95%, or 99% sequence identity to one of SEQ ID NO:3, SEQ ID NO:2, SEQ ID NO:1, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, and/or SEQ ID NO:23.

27. The method of claim 26, wherein the polypeptide is vitronectin or a fragment of vitronectin.

28. The method of claim 26 or claim 27, wherein the immune cell is a mammalian immune cell.

29. The method of claim 28, wherein the mammalian immune cell is a human immune cell.

30. The method of any one of claims 24 to 29, wherein the cell for adoptive cell therapy is a CAR expressing cell.

31. The method of claim 30, wherein the CAR expressing immune cell is a CAR T-cell or CAR-Natural Killer (CAR-NK) cell.

32. The method of any one of claims 25 to 31, further comprising the step of harvesting one or more transduced cells.

33. The method of any one of claims 25 to 32, further comprising the step of expanding the transduced cells.

34. The method of any one of claims 25 to 33, further comprising formulating transduced mammalian cells with a pharmaceutically acceptable excipient to generate a medicament.
